(19) United States
(12) Patent Application Publication
(10) Pub. No.: US 2004/0132025 A1
(43) Pub. Date: Jul. 8, 2004

(54) Ampk-related serine/threonine kinase, designated snark

(76) Inventors: Daniel J. Drucker, Toronto (CA); Cherly F. Rosen, Toronto (CA); Diana L. Lefebvre, Mississauga (CA)

Correspondence Address: ROTHWELL, FIGG, ERNST & MANBECK, P.C., 1425 K STREET, N.W., SUITE 800, WASHINGTON, DC, 20005, US

(21) Appl. No.: 10/343,514

(22) PCT Filed: Aug. 2, 2001
(86) PCT No.: PCT/CA01/01109

(30) Foreign Application Priority Data
Mar. 28, 2001 (CA) 2,340,783

(51) Int. Cl. C12Q 1/68; C07H 21/04; C12N 9/12

(52) U.S. Cl. 435/6; 435/69.1; 435/194; 435/320.1; 435/325; 536/23.2

(57) ABSTRACT

The cloning and function of a new AMPK-related kinase, designated SNARK, are described. The kinase SNARK is involved in the stress response to glucose deprivation. Provided are the rodent and human genes encoding SNARK, and the SNARK protein and useful fragments, in isolated form. Also provided are SNARK expression systems and assays useful to identify SNARK substrates and SNARK activity modulators, and antibodies useful as SNARK modulators for instance in therapeutic applications to modulate the metabolism of glucose.

### FIELD OF THE INVENTION

This invention is in the field of molecular biology and the emerging field of proteomics. It relates more particularly to certain novel protein kinases, and their applications in drug discovery and medical diagnostics.

### BACKGROUND TO THE INVENTION

Protein kinase cascades are highly conserved between animals, fungi and plants. The Sucrose-Non Fermenting protein kinase (SNF1) from Saccharomyces cerevisiae and its mammalian counterpart, AMP-activated protein kinase (AMPK), form a family of serine/threonine kinases and represent key components in yeast and mammalian stress response systems [1]. This family of kinases is commonly activated in response to cellular and environment stresses, including nutrient deprivation. SNF1 responds to glucose deprivation by derepressing genes implicated in carbon source utilization and by modulating transcription of glucose-regulated genes involved in gluconeogenesis, respiration, sporulation, thermotolerance, peroxisome biogenesis, and cell cycle regulation [2]. AMPK is similarly activated by environmental stresses that result in increases in the cellular ATP:AMP ratio. Activated AMPK switches off anabolic pathways (e.g. fatty acid and cholesterol synthesis) and induces ATP-generating catabolic pathways (i.e. fatty acid oxidation) [1].

The SNF1/AMPK family of serine/threonine kinases has expanded rapidly following the cloning of several SNF1/AMPK-related protein kinases in plants [3-11], Plasmodium falciparum[12], Chlamydomonas [13] and mammals [14-19]. These protein kinases have been assigned to the SNF1/AMPK family primarily on the basis of their structural similarity with the catalytic domains of SNF1 and AMPK. The available structural and functional data is consistent with the notion that SNF1, AMPK and related kinases represent components of signaling cascades that control metabolism, gene expression and perhaps cell proliferation in response to cellular, metabolic and environmental stress [20].

In general, it is known that the chemical AICAR (5-aminoimidizole-4-caroximide riboside) activates AMPK, and a downstream set of metabolic responses that may be insulin-like in certain tissues such as muscle and liver. The AMPK system seems to function as a cellular fuel gauge, monitoring the energy state of the cell and protecting the cell from energy depletion. It is not clear in the literature that AMPK is responsible for all of the downstream events detected following AICAR treatment of cells. The system is often studied by treating cells with AICAR, which mimics the effects of AMP on the AMPK system. AICAR was previously presumed to be relatively specific for activation of AMPK. Given the central importance of the AMPK cascade in fuel metabolism and energy balance, and given the numerous medical conditions and disorders that manifest from an imbalance in this cascade, it would be desirable to expand the understanding of those components that are critical for its proper functioning.

### SUMMARY OF THE INVENTION

There has now been identified a novel mammalian member of the kinase family, designated SNARK, having significant homology to the catalytic domain of the SNF1/AMPK family of serine threonine protein kinases. Results presented herein identify SNARK as a novel glucose- and AICAR-regulated mammalian member of the AMPK-related kinase gene family, and confirm that SNARK represents a new candidate mediator of the cellular responses to metabolic stress.

In one of its aspects, the present invention provides an isolated polynucleotide, in the form of RNA or DNA, including cDNA and genomic DNA and synthetic equivalents thereof, that encodes a SNARK protein which is the SNARK protein of SEQ ID NO. 1, or a mammalian homolog thereof including the human SNARK protein encoded within human chromosome 1q32, or a variant or chimeric SNARK protein that retains SNARK activity and shares at least 70%, e.g. 80% and more preferably at least 90% e.g. 95-99%, sequence identity with the SNARK protein of SEQ ID NO. 1.

Also provided by the present invention are polynucleotides and oligonucleotides that hybridize with the SNARK-encoding polynucleotides. Such hybridizing poly- and oligonucleotides are optionally detectably labeled, and have a sequence complementary to, or an anti-sense version of, a characterizing region of the SNARK-encoding polynucleotides. Such hybridizing poly- and oligonucleotides are useful to detect SNARK-encoding polynucleotides in a given sample, for instance to probe for or amplify SNARK-encoding mRNA or DNA in a library, or to identify SNARK-encoding polynucleotide in a given tissue sample by in situ localization. Such hybridizing poly- and oligonucleotides are useful also to arrest endogenous expression of the SNARK-encoding polynucleotide, for instance to modulate SNARK production for therapeutic intervention. In embodiments of the invention, the oligonucleotides are designed to bind to the polynucleotide region encoding the C-terminal region of the SNARK protein, which among other members of the SNF1/AMPK family, comprises unique protein sequence distinctive of SNARK. Thus, in embodiments of the invention, there are provided oligonucleotides that hybridize under stringent conditions with that region of SNARK-encoding DNA that codes for a unique C-terminal region thereof, such as the region constituted by amino acids 310 through 630 of SEQ ID NO. 1 or the comparable region of a homolog thereof, or a sub-region comprising at least about 20 nucleotides, e.g., desirably about 40 nucleotides, thereof.

The polynucleotides of the present invention are useful, in another aspect, for expression to produce SNARK protein in isolated form, or as a protein conjugate. Accordingly, there are provided vectors that incorporate the SNARK-encoding polynucleotides in operable combination with expression controlling elements for driving the expression thereof in a suitable host. In related aspects of the invention, there are provided cellular hosts incorporating the expressible, SNARK-encoding polynucleotides. Also provided are methods for SNARK production, which comprises the step of culturing SNARK production hosts under conditions adapted for producing SNARK. In a further related aspect, the SNARK production hosts are useful to screen for modulators of SNARK activity, thereby to identify agents useful to modulate SNARK activity either in vitro, or in vivo for therapeutic purposes.

Included among the oligonucleotides useful as probes to identify SNARK homologs are the human EST sequences reported in the BLAST database as having homology with a region of SEQ ID NO. 2 that is at least about 85%. Such oligonucleotides include those referenced as gb/AI469033.1/AI469033 (ti70a02.x1) reported as NCI_CGAP_Kid11 (which scores 525 bits at an E value of e-146); and as gb/AA995360.1/AA995360 (or74b03.s1) reported as NCI_CGAP_Lu5 (which scores 426 bits at an E value of e-117). The present invention thus embraces the human homolog of SNARK, which human homolog incorporates amino acid sequence that is encoded by such human EST sequences. The present invention further embraces polynucleotides that encode the human homolog of SNARK, and incorporates amino acid sequence encoded by such ESTs or sequence having at least about 95% identity therewith as exemplified further herein. In a related aspect, the present invention further provides a method for detecting SNARK-encoding DNA polynucleotide in a sample, in which such human ESTs, and extended or fragmented forms thereof are used optionally in labeled forms as probes.

In another of its aspects, the present invention provides SNARK protein, in isolated form, and optionally incorporating a detectable label. Such SNARK protein may be in the form of the rat SNARK protein of SEQ ID NO. 1, a mammalian homolog thereof including human SNARK and mouse SNARK, variants of such mammalian forms of SNARK, and chimeric forms thereof in which regions or domains thereof, such as the catalytic domain, have been exchanged. The SNARK protein may further comprise a carrier useful, for instance, to raise antibodies thereto. Alternatively, the SNARK protein may be combined with a pharmaceutically acceptable carrier, for use as a therapeutic.

Also provided by the present invention are SNARK fragments, e.g., having N- and/or C-terminal truncations, including for instance an immunogenic fragment against which antibodies can be raised, or comprising a region capable of inhibiting the binding of SNARK with its binding partners that participate in the signaling cascade in which SNARK is involved endogenously, thereby to downregulate SNARK activity. Such immunogenic fragments comprise at least about 20 amino acids, and preferably incorporate a contiguous portion of the C-terminal SNARK region spanning residues 310 through 630 of SEQ ID NO. 1 or a corresponding region of a mammalian homolog of SEQ ID NO. 1., or a variant of such region having one or more, e.g. up to about 10, conservative amino acid substitutions.

Also provided, in another aspect of the present invention, are antibodies that bind selectively to SNARK,e.g., with preference relative to other AMP kinases. In embodiments, these SNARK-binding antibodies are in detectably labeled form, to allow for the detection of SNARK in a given sample. Alternatively, the SNARK antibodies are used to modulate SNARK activity either in vivo, for therapeutic purposes, or in vitro, for drug screening and related investigational purposes.

In another of its aspects, the present invention provides a method for assaying SNARK activity, the method comprising the step of obtaining a candidate SNARK protein, incubating the SNARK protein with a SNARK substrate under phosphorylating conditions, and then determining whether phosphorylation has occurred, wherein phosphorylation reveals the SNARK candidate protein has SNARK activity. In a related aspect of the invention, the assay is modified to identify SNARK activity modulators, in which SNARK protein, a selected SNARK substrate and a candidate modulator of SNARK activity are incubated under phosphorylating conditions, and determining the extent of phosphorylation in the presence of the candidate modulator relative to the extent of phosphorylation in the absence of the candidate modulator, wherein modulating activity is revealed by a difference in phosphorylation in the presence of the modulator relative to the absence of the modulator.

These and other aspects of the invention are described in greater detail with reference to the accompanying drawings, in which:

### REFERENCE TO THE DRAWINGS

FIG. 1(A). Southern blot analysis of genomic DNA isolated from rat liver and human lymphocytes. Genomic DNAs (15 μg) were digested with one of the restriction endonucleases BamHI, EcoRI or HindIII. The blot was hybridized with a 1.5 kb fragment (≈nt 1400-2929) corresponding largely to the 3′-end of the SNARK protein. The approximate positions of the DNA size markers are indicated at the right. (B). Chromosomal localization of human SNARK. A 450 bp fragment of the rat UV126 cDNA [20] was used as a probe to screen a P1-derived artificial chromosome (PAC) library. The probe identified one genomic PAC as positive and this PAC was mapped to human chromosome 1q32. Positive hybridization signals at 1q32 (seen as bright spots on the chromosome) were noted on both homologues in >90% of the cells.

FIG. 2. Nucleotide and deduced amino acid sequences of rat SNARK. The deduced amino acid sequence of SNARK (SEQ ID NO. 1) is shown in single-letter code above the respective coding nucleotide sequence (SEQ ID NO. 2). Nucleotide number assignment is listed on the right and amino acid number assignment is shown as underlined numbers on the right. The protein serine/threonine kinase catalytic domains are boxed. The protein kinase ATP-binding region signature is underscored with a dotted line. The serine/threonine kinase active-site signature is underlined with a dashed line.

FIG. 3. Alignment of the deduced amino acid sequence of rat SNARK and other members of the SNF1/AMPK family using the CLUSTAL W algorithm. The protein kinase catalytic domains are boxed. Identical residues are indicated by asterisks and conservative substitutions are indicated by dots under the sequences (‘:’ indicates substitution with a strong group, score >0.5, and ‘.’ indicates a substitution with a weak group, score ≦0.5). The amino acid residues are numbered on the right.

FIG. 4(A). Northern blot analysis of rat tissues showing tissue distribution of SNARK mRNA. Ten μg of total RNA isolated from each rat tissue (indicated above the appropriate lane) was electrophoresed, transferred to a nylon membrane and probed with a fragment of the SNARK/pcDNA3.1 corresponding to the protein coding region (nt 0-1975) and exposed to film. Positions of the 28S and 18S ribosomal RNA are indicated at the right of the autoradiograph. (B). Reverse transcriptase PCR analysis of SNARK in various rat tissues. The upper panel shows an ethidium bromide stained gel of RT-PCR products resulting from first-strand cDNAs prepared from rat heart (lane 1), skin (lane 2), spleen (lane 3), kidney (lane 4), lung (lane 5), liver (lane 6), uterus (lane 7), testis (lane 8) and NRKC cells (lane 9). A negative control reaction containing no first-strand DNA was included to verify specificity of primer products (lane 10). The lower panel shows a Southern analysis of the RT-PCR products resulting from each tissue type. The Southern blot was hybridized with a fragment of SNARK corresponding to its protein-coding region (nt 0-1975). Positions of migration of the DNA size markers are shown on the right of the figure.

FIG. 5(A) Analysis of SNARK protein transcribed and translated in rabbit reticulocyte lysate. In lanes 1 and 2, one-tenth of the TNT reaction was loaded directly onto a 8% polyacrylamide-SDS gel. Lanes 3-5 are size-fractionated immunoprecipitation (IP) reactions. Lanes 3 and 4 are control IP reactions where no TNT products or T7-luciferase TNT products were incubated with SNARK antiserum #14, respectively. Lane 5 shows SNARK TNT immunoprecipitated with SNARK antiserum. Following gel electrophoresis, the SDS-polyacrylamide gel was fixed, dried and exposed to BioMax MS film with intensifying screen for 4 hours. (B) Western analysis of SNARK protein in stably transformed BHK cells. NRKC cell extract (750 μg; lane 1) or 500 μg of BHK+1 (lanes 2 and 3) and BHK+11 (lane 4) were immunoprecipitated with SNARK antiserum #14 (lanes 1, 2 and 4) or with nonimmune serum (lane 3), electrophoresed on a 8% polyacrylamide-SDS and transferred onto a PVDF membrane. Western analysis was performed using the SNARK antiserum #16. The positions of the protein standards are shown at the right in kDa. The position of the SNARK protein is listed at the left of the autoradiograph.

FIG. 6. Autophosphorylation of SNARK. SNARK was immunoprecipitated from 500 μg of wildtype BHK, BHK+1 and BHK+11 cell extract with either SNARK antiserum #14 (lanes 1, 2, 4 and 6) or nonimmune serum (lanes 3 and 5). Lane 6 is a negative immunoprecipitation control containing 500 μg of BHK+1 cell extract but no antiserum. Immunoprecipitates were incubated with [γ]-ATP³² at 30° C. and the reactions were stopped after 30 minutes by the addition of 2X SDS loading buffer and boiling for 5 minutes. Samples were electrophoresed on a 8% polyacrylamide-SDS gel and the gel was dried and exposed to BioMax MS film.

FIG. 7. The SNARK protein possesses AMPK-like phosphotransferase activity. SNARK protein was immunoprecipitated from 500 μg of cell lysate from wildtype BHK, BHK−1, BHK+1 and BHK+11 cells. Kinase assays were performed using the 200 μM SAMS peptide as substrate in kinase reaction cocktail. NRKC cells (stippled box); wildtype BHK cells (solid box); BHK+1 and +11 cells (hatched boxes). Results are the means±S.E.M. for 2 experiments (n=5 each time). For comparative purposes, AMPKα2 activity was assayed in these cell lines and found to be equivalent to, 4-fold lower and 1.5-fold lower than the SNARK activity levels measured under basal growth conditions in wildtype BHK, SNARK-transfected BHK and NRKC cell lines, respectively (data not shown).

FIG. 8.(A) Activation of SNARK in NRKC cells using AICAR. NRKC cells were treated with 0, 0.5 mM, 1 mM or 2 mM AICAR for 1 hour. SNARK protein was immunoprecipitated from 500 μg of cell lysates with SNARK antiserum and kinase assays were performed in the presence of 200 μM AMP using the SAMS peptide. Solid box represents control (0 mM AICAR) samples and hatched boxes represent AICAR-treated samples. Data is expressed as phosphotransferase activity relative to control values (control=1) and represents the means±S.E.M. for 2 individual experiments with at least 10 samples per group per experiment. *p<0.06, relative to control values. Using the same assay conditions, basal AMPK-α2 activity was found to be 1.5-fold lower than SNARK activity and increased at least 2-fold upon treatment with ImM AICAR in NRKC cells (data not shown). Basal AMPKα1 activity was found to be 88 times higher than basal SNARK activity, but was not stimulated by treatment with AICAR (data not shown). (B) Activation of SNARK in wildtype BHK cells resulting from glucose deprivation. Wildtype BHK cells were exposed to glucose-free medium for 0 or 90 minutes. SNARK was immunoprecipitated and kinase assays were performed using the SAMS peptide as substrate in the presence of AMP Solid box represents control (25 mM glucose) samples and hatched boxes represent glucose-deprived samples. Results are expressed as phosphotransferase activity relative to control values (control=1) and represents the mean±S.E.M. from 2 individual experiments with at least 10 samples per group per experiment. *p<0.03, relative to control values. Basal AMPKα2 activity levels measured in wildtype BHK cells were comparable with basal SNARK activity detected in these cells (data not shown).

FIG. 9 compares, at the amino acid level, the 1-251 region of rat SNARK with (A) the amino acid sequence encoded by a human polynucleotide and (B) the amino acid sequence of a mouse polynucleotide, both of which were identified by in silico screening. Provided at the top of each Figure are the accession numbers for the identified homologs, and corresponding literature citations where available.

FIG. 10 provides a comparison of rat SNARK-encoding DNA with polynucleotides of human genomic, RNA or cDNA origin identified in silico by searching (1) with the complete sequence of rat SNARK-encoding cDNA, including (A) correlations with the human genome database, with sequence gaps determined by sequencing of SNARK-encoding DNA isolated from the human cell line HaCaT, (B) correlations with the human mRNA database; and (2) with the amino acid sequence of rat SNARK protein including (C) correlations with the human EST database. FIG. 10 (D) provides a comparison of rat SNARK encoding DNA with the sequence encoding human SNARK cloned from the HACAT cell line. Provided at the top of each Figure are the accession numbers for the identified homologs, and corresponding literature citations where available.

FIG. 11 provides a comparison of rat SNARK-encoding DNA with polynucleotides of mouse genomic, RNA or cDNA origin identified in silico by searching (1) with the complete sequence of rat SNARK-encoding cDNA, including (A) correlations with the mouse high throughput genome database; (B) correlations with the mouse nr database; and (2) with the amino acid sequence of rat SNARK protein including (C) and (D) correlations with the mouse EST database. FIG. 11(E) provides a comparison of rat SNARK encoding DNA with the sequence encoding mouse SNARK cloned from hairless mice. Provided at the top of each Figure are the accession numbers for the identified homologs, and corresponding literature citations where available.

### DETAILED DESCRIPTION OF THE INVENTION AND PREFERRED EMBODIMENTS

The present invention relates to a novel mammalian form of a kinase related to the family of SNF1/AMPK serine/threonine protein kinases, which has been designated SNARK. In embodiments, the SNARK protein is provided in “isolated” form, i.e., in a form essentially free from proteins with which that form of SNARK is normally associated.

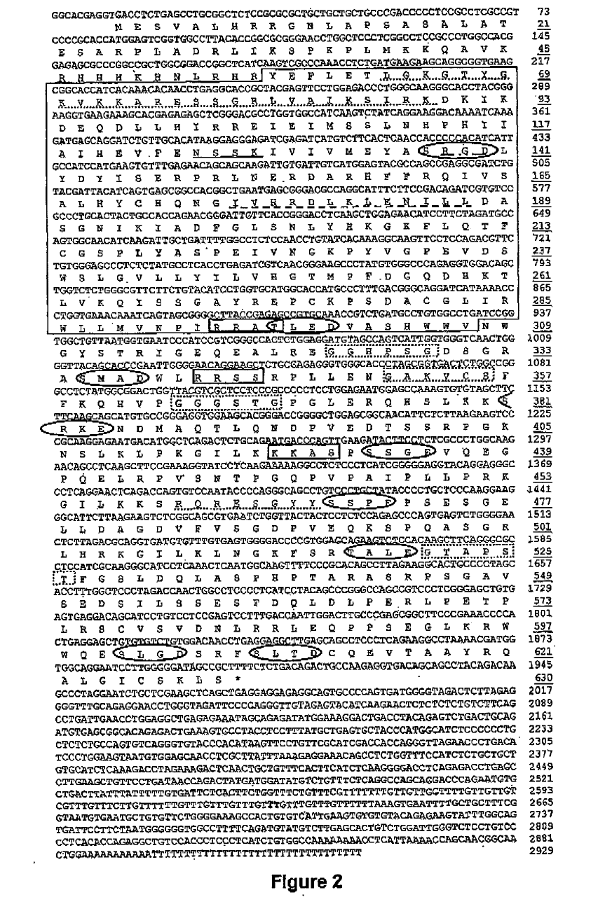

As illustrated in FIG. 2, the rat form of the SNARK protein comprises 630 amino acids [SEQ ID NO. 1]. Identified on FIG. 2 are consensus regions indicating the protein serine/threonine kinase catalytic domains (boxed), the protein kinase ATP-binding region signature (underscored with a dotted line), and the serine/threonine kinase active-site (underlined with a dashed line).

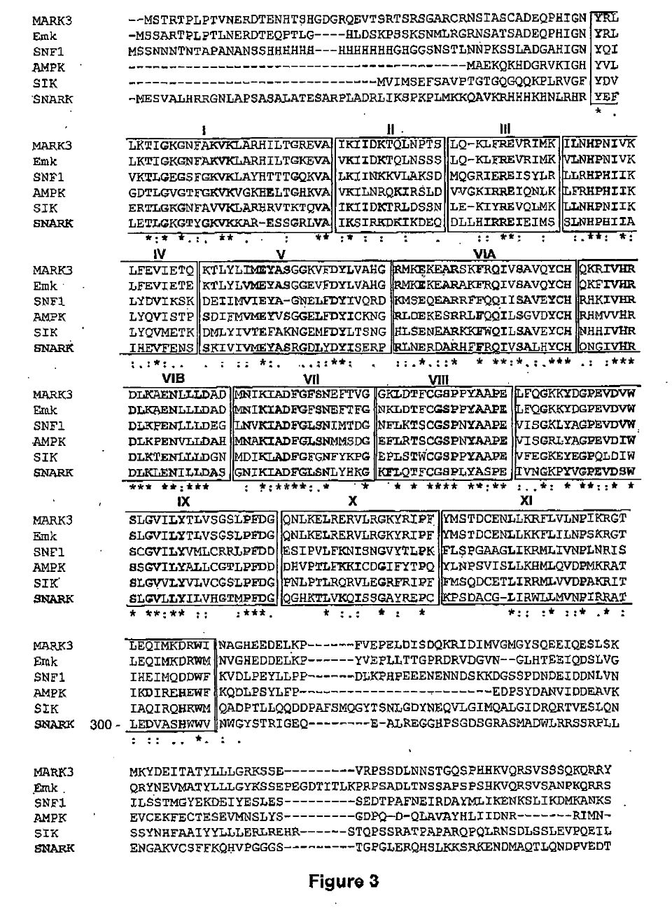

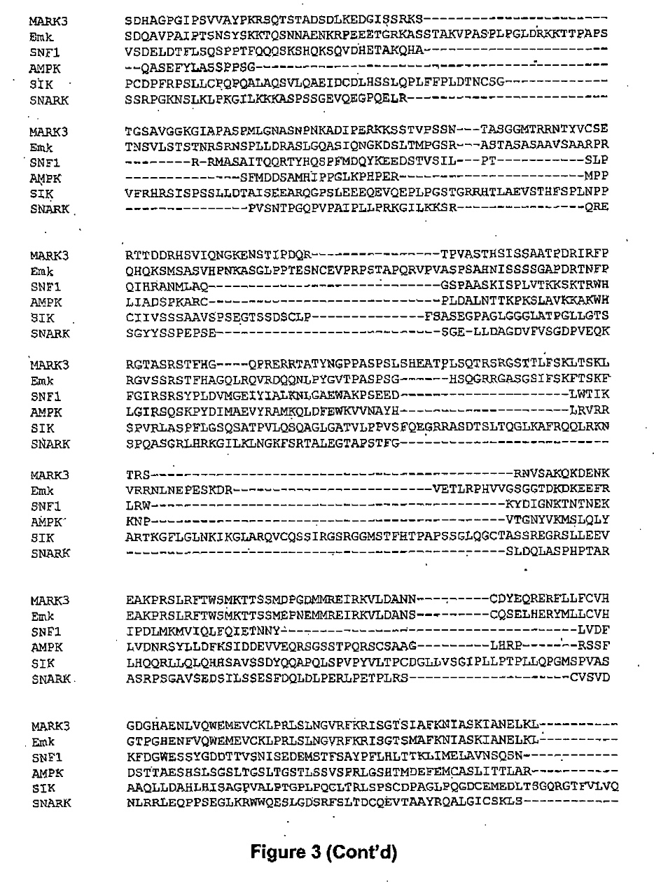

The alignment of the deduced amino acid sequence of rat SNARK and other members of the SNF1/AMPK family using the CLUSTAL W algorithm is shown in FIG. 3. The protein kinase catalytic domains are boxed. Asterisks indicate identical residues and conservative substitutions are indicated by dots under the sequences. The amino acid residues are numbered on the right. Accession numbers of the respective comparison sequences are: p78 (PIR, s27966), emk (PIR, s31333), SIK (gb, AB020480), SNF1 (PIR, a26030) and AMPK (gb, z29486).

Thus, in one embodiment, the invention encompasses the SNARK protein identified by SEQ ID NO. 1. The invention also encompasses SNARK homologs, including the human homolog and the mouse homolog, and variants and chimeric forms of SNARK, which retain functional activity of SNARK. Variants of SNARK include SNARK proteins that differ relative to a SNARK homolog by incorporating amino acid substitutions, insertions, or deletions that do not disrupt SNARK function, such as SNARK phosphorylating activity. Generally, such alterations will not affect more than about 10% of the primary structure of SNARK. For instance, amino acid substitutions, deletions or insertions will not generally involve more than about 20 amino acids, e.g., more than about 10 amino acids. In embodiments of the invention, the SNARK homolog or variant shares at least 70%, e.g., at least 80% identity to the SNARK having SEQ ID NO. 1. The C-terminal region of SNARK is a particularly unique region of the protein, having almost no homology in its 310-630 region with the similarly positioned regions of other members of the SNF1/AMPK family, as shown in FIG. 3. Accordingly, in a preferred embodiment, variants of the SNARK protein share at least 90% identity, and more preferably at least 95%, e.g., 98%-99% identity, with this 310 -630 region of SNARK. In this context, a preferred SNARK variant is one having the noted identity with the C-terminal SNARK region, and an overall identity of at least 80%, and more preferably 90%, amino acid sequence identity to the SNARK of SEQ ID NO. 1. A most preferred SNARK variant is one having at least 95% amino acid sequence identity thereto. It is recognized that mammalian SNARK proteins may exhibit a greater degree of amino acid variation in the C-terminal region of the SNARK protein, compared to the more conserved functional N-terminal kinase domains.

Proteins that exhibit “SNARK activity” are defined as those proteins that (1) are recognized by antisera against native rodent or human SNARK, (2) exhibit phosphorylating activity against a synthetic or natural substrate, such as SAMS, that is also recognized by native rodent or human SNARK, and (3) exhibit the following rank order of substrate selectivity: SAMS>MBP>B-casein>whole histone fraction>protamine sulfate. Such proteins, that exhibit SNARK activity can be further characterized structurally as exhibiting at least 75% amino acid identity, and more desirably at least 90% identity within the conserved functional kinase domains of rat SNARK as outlined in FIG. 2 and 3.

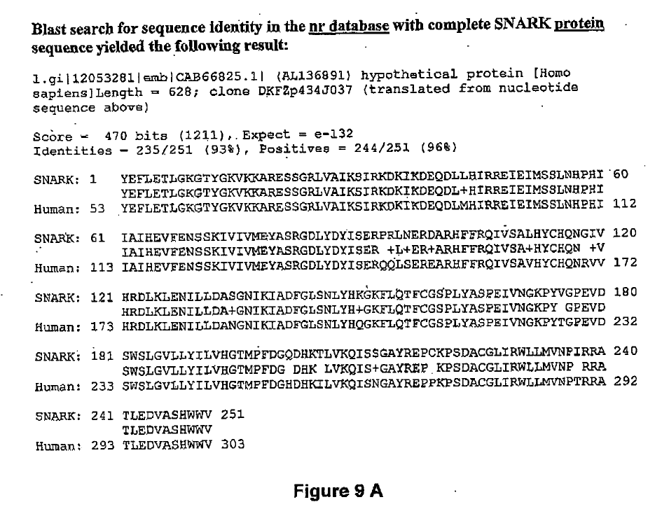

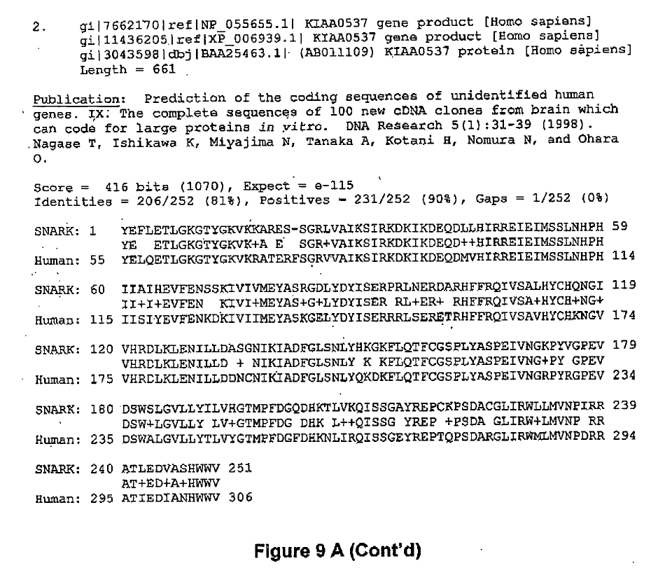

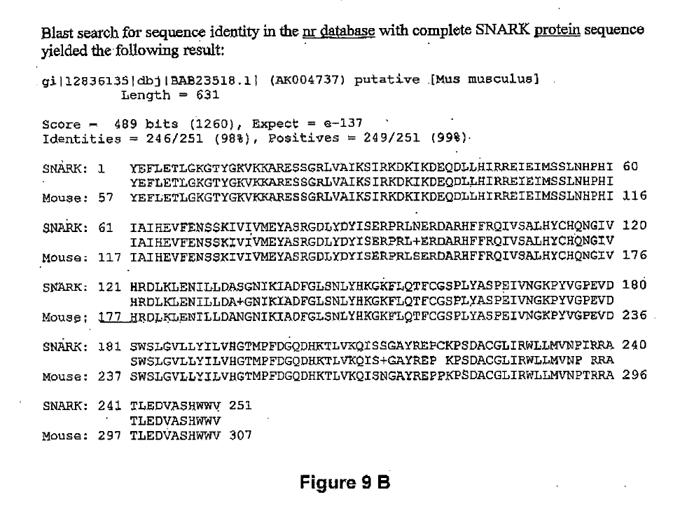

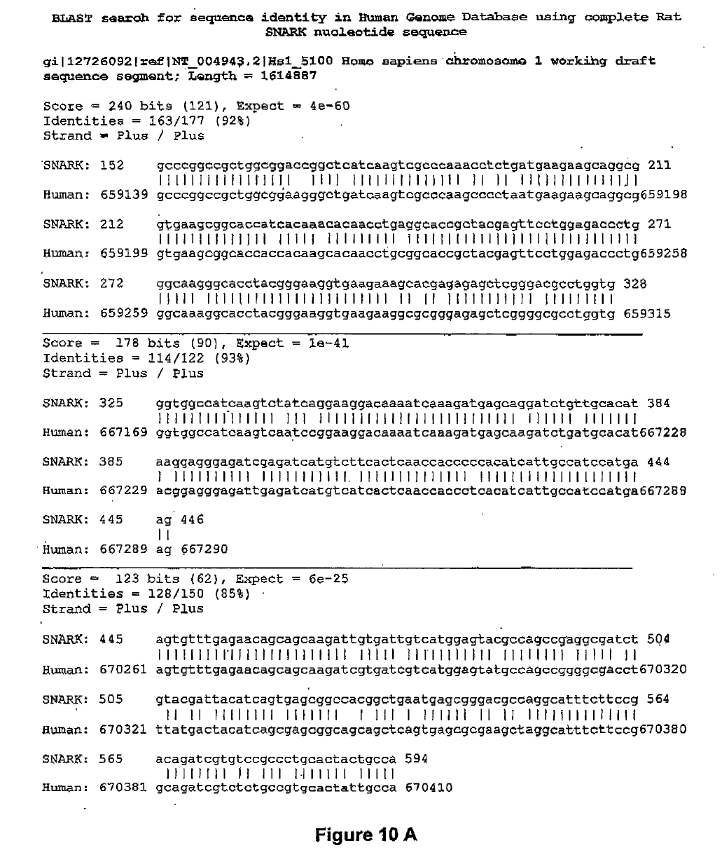

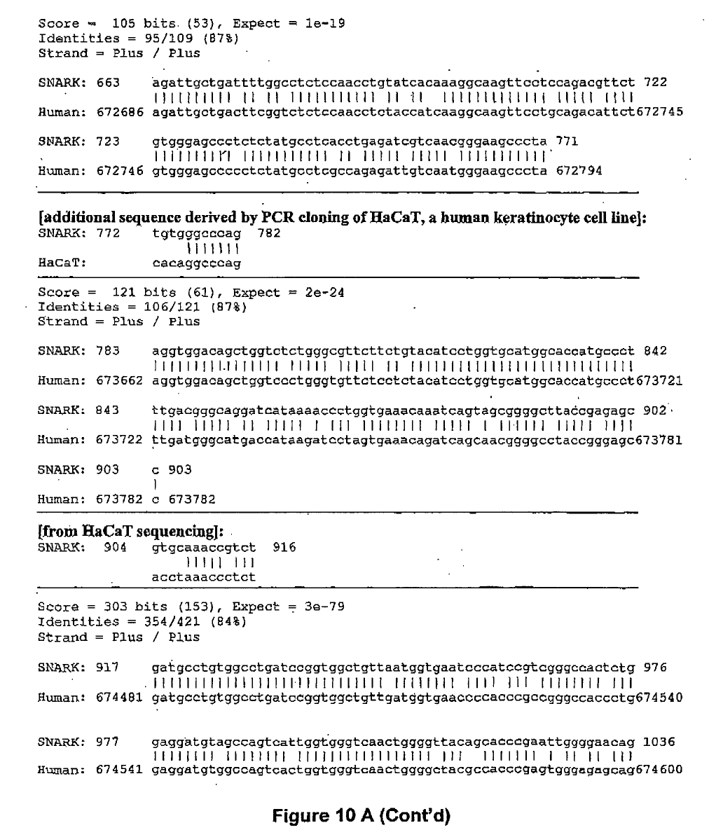

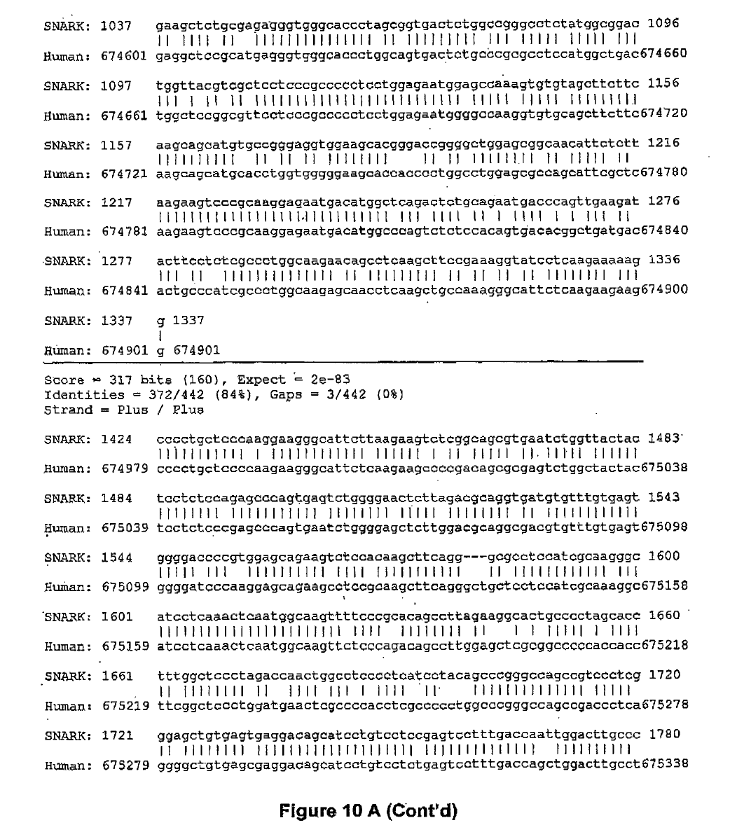

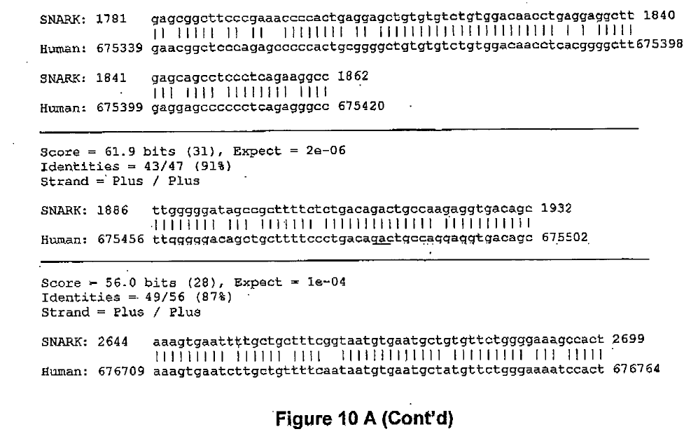

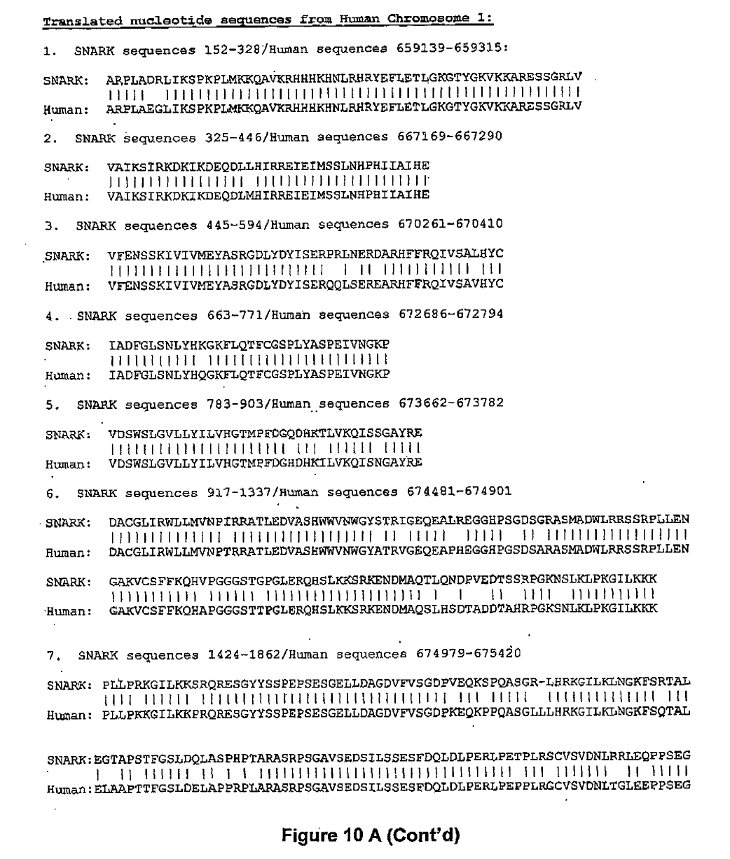

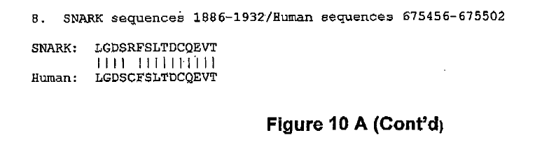

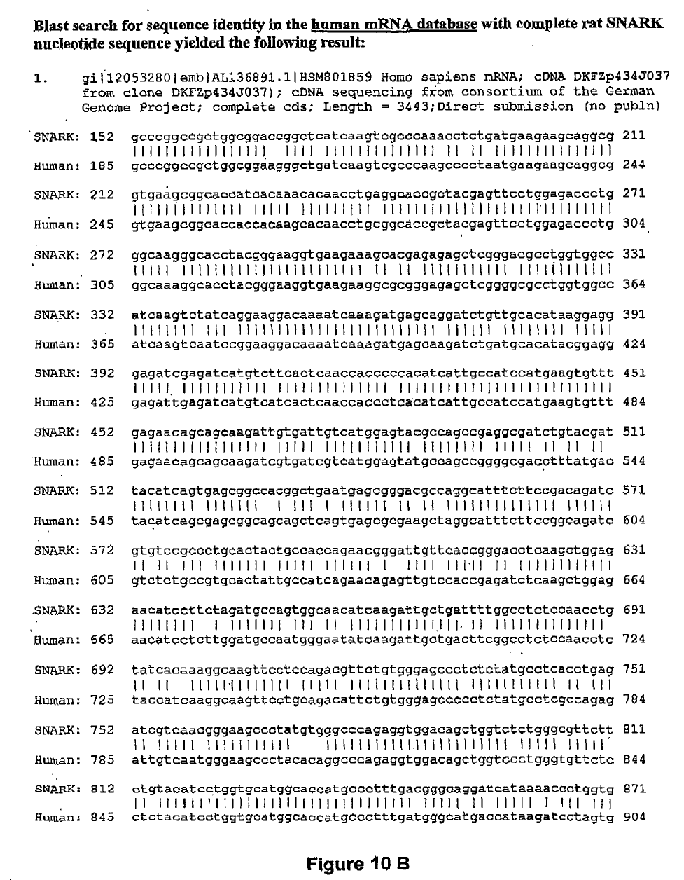

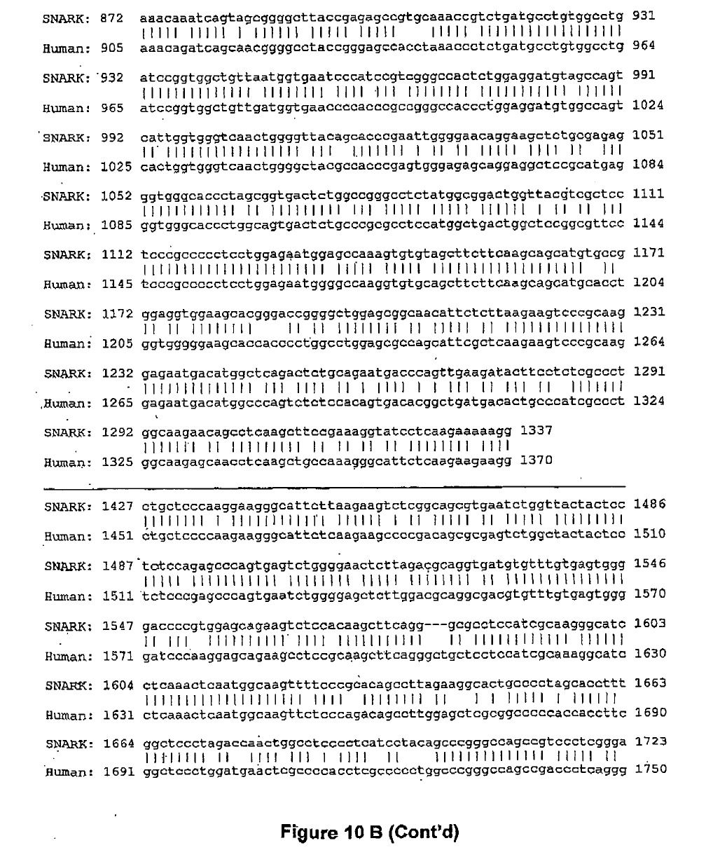

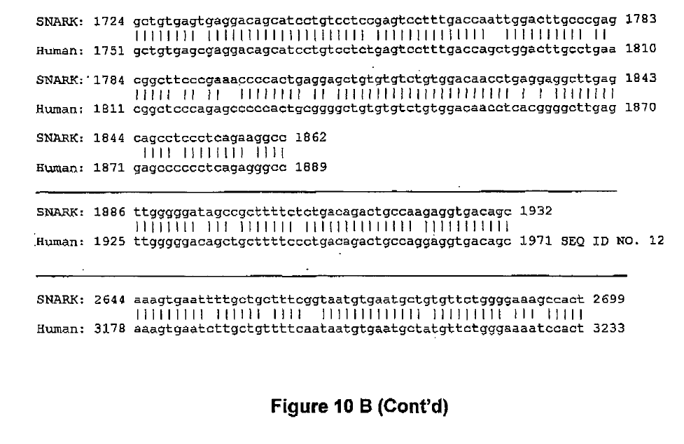

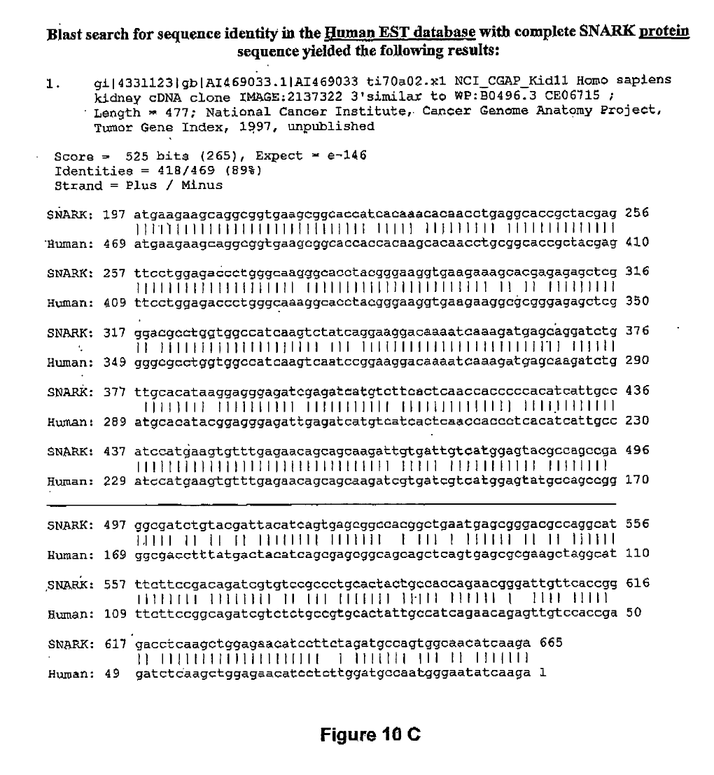

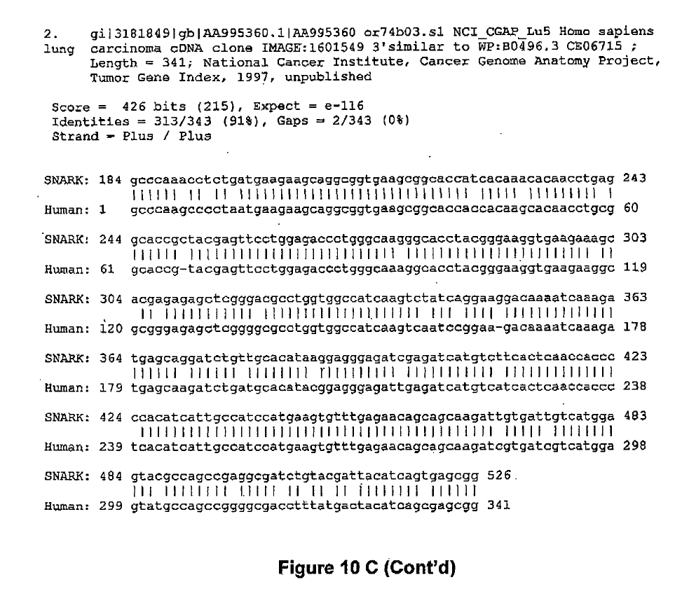

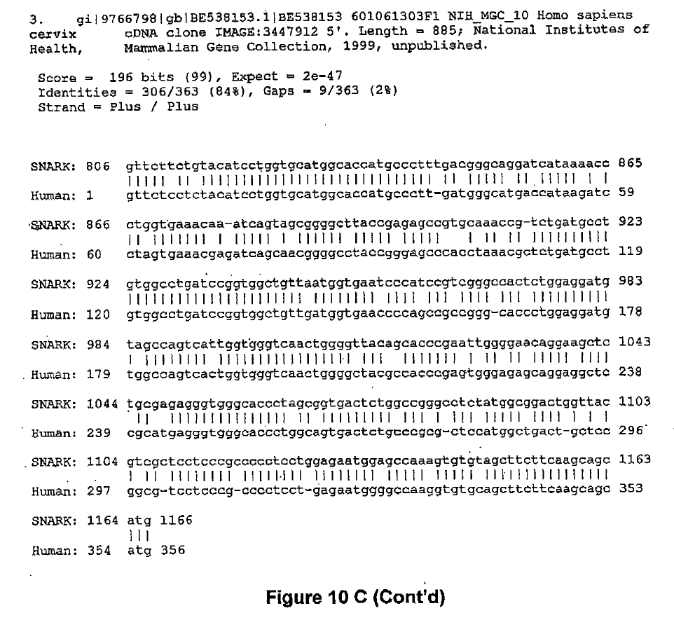

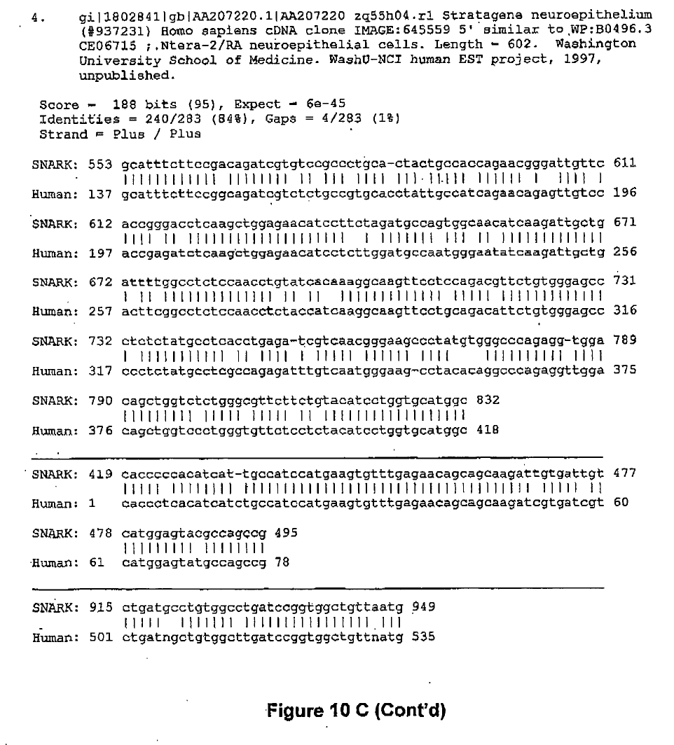

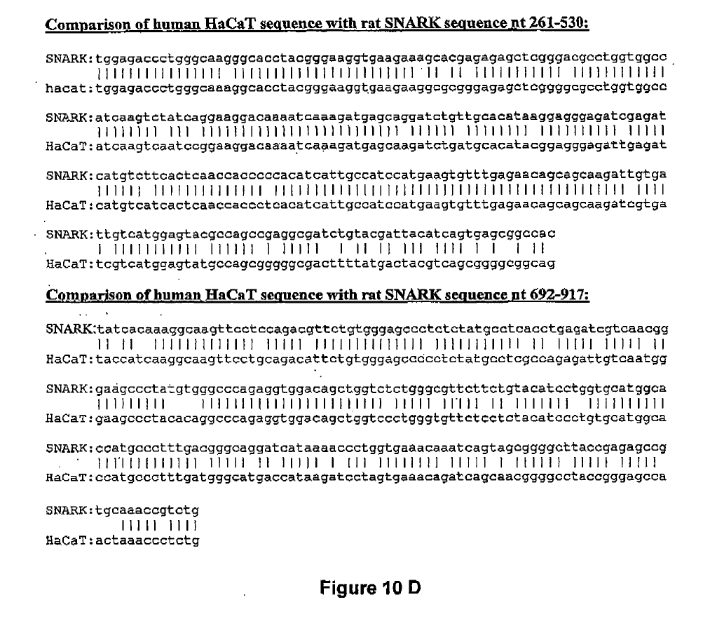

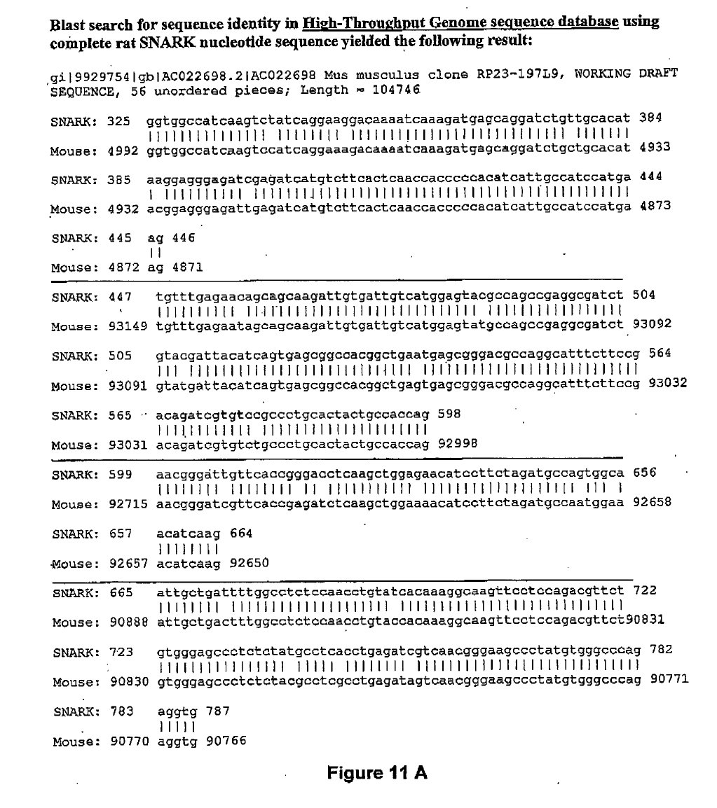

In embodiments of the present invention, the SNARK protein is a human homolog of SEQ ID NO. 1. In embodiments, the human homolog incorporates amino acid sequence encoded by highly homologous (Value>200) EST and genomic clones identifiable in the public BLAST or similar database upon searching against the rat SNARK DNA of SEQ ID NO. 2. Examples of such human EST's are those reported in GenBank as accession numbers AI469033.1 and AA995360.1, and available as IMAGE clones 2137322 and 1601549, respectively. The sequences of such clones are mapped onto SNARK-encoding DNA, in FIG. 10. In a particular embodiment of the invention, the SNARK protein is a human homolog that incorporates, within its overall sequence, the 251 amino acid sequence depicted in FIG. 9A, or a variant thereof incorporating amino acid alteration(s) that does not disrupt SNARK activity. In other specific embodiments, the human SNARK protein incorporates amino acid sequences that are encoded by one or more of the coding regions of the polynucleotides shown in FIGS. 10A, 10B, 10C and 10D.

The human SNARK protein is characterized by encoded gene sequences that are identified in public domain or proprietary databases, and is recognized by antisera directed at conserved domains within rodent and human SNARK. The human SNARK protein is expected to exhibit 75%, and suitably, 90% amino acid identity with rodent SNARK at key functional kinase domains, as exemplified by sequence alignment shown in FIG. 9. A human SNARK protein also exhibits the same degree of substrate specificity exhibited by rat SNARK, as shown in Table 1, infra.

Obtaining of the nucleotide sequence of an intact and full length cDNA encoding such a human homolog is alternatively achieved by screening a suitable cDNA library, such as a cDNA library generated from keratinocytes obtained for instance from skin or muscle tissue such as heart, or from the HaCaT cell line in the manner exemplified herein. The tissue desirably is first irradiated in the manner reported by Rosen et al [20], to induce human SNARK expression. Screening of the library is achieved either by labeled probing with the SNARK SEQ ID NO. 2, or with a sequence corresponding to the ESTs and genomic clones just described, or with any major hybridizing fragment thereof. Alternatively, the human SNARK homologue may be obtained by using the rat SNARK sequence provided herein to screen human DNA databases to identify previously unknown nucleotide sequences that can be identified as human SNARK homologues. As one example of this possibility, we identify human EST's reported in GenBank as accession numbers AI469033.1 and AA995360.1, that correspond to cDNAs encoding partial human SNARK coding sequences. The remainder of the human SNARK sequences can be obtained by cDNA cloning, RT-PCR, or further additional database searches, using the information provided herein. More particularly, the sequences encoding regions of human SNARK are provided in FIG. 10.

Desirably, the SNARK variants retain the property of autophosphorylation possessed by SNARK. Such SNARK activity can be assessed using the autophosphorylation assay herein described.

As noted, the invention further embraces homologs of the SNARK protein identified in SEQ ID NO. 1 including the human homolog encoded on chromosome site 1q32, and other mammalian homologs encoded by polynucleotides that hybridize under stringent conditions with the SNARK of SEQ ID NO. 2.

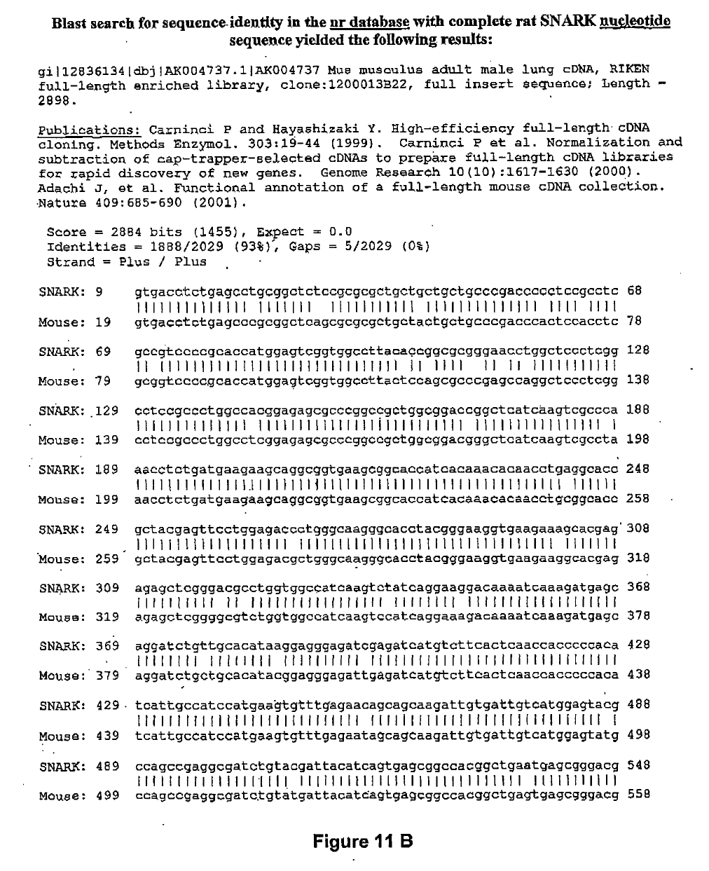

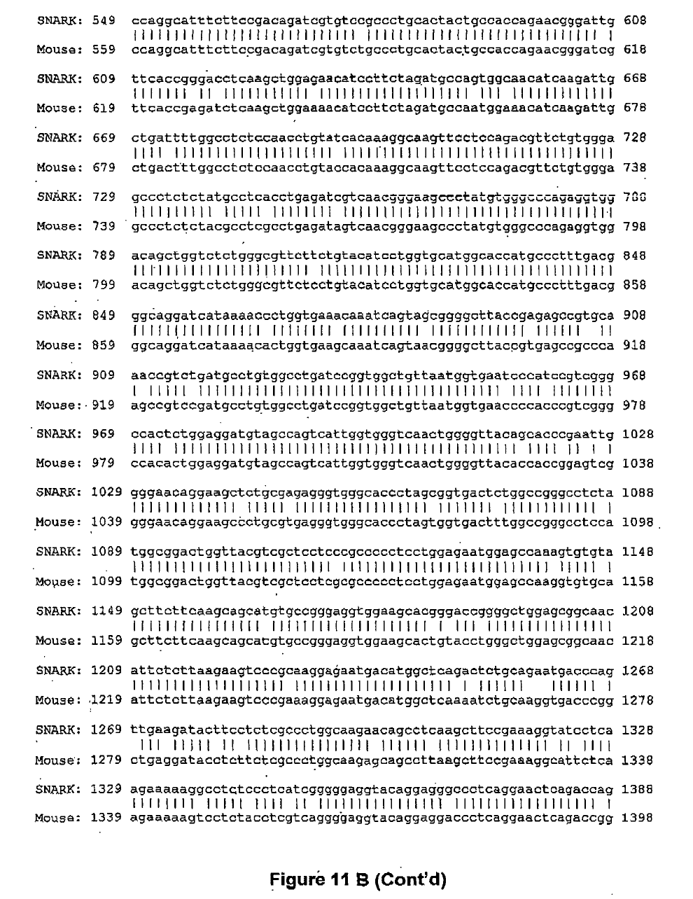

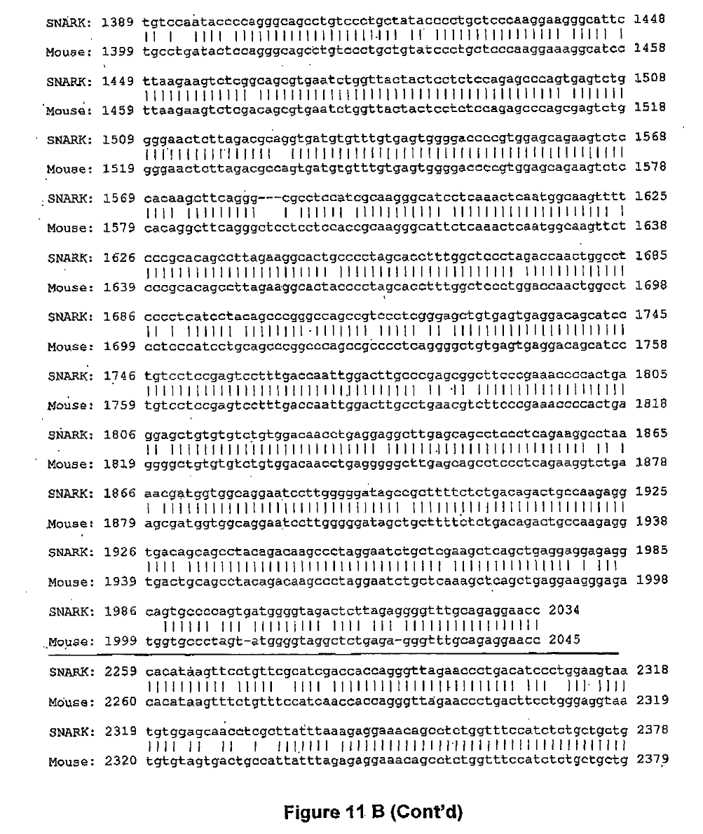

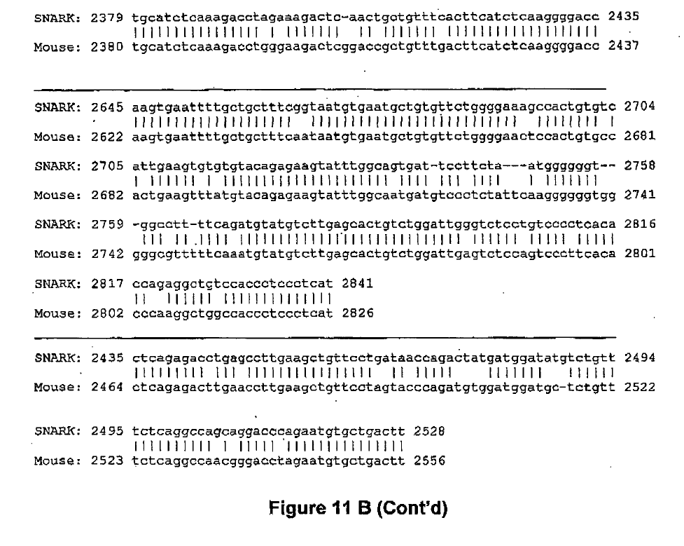

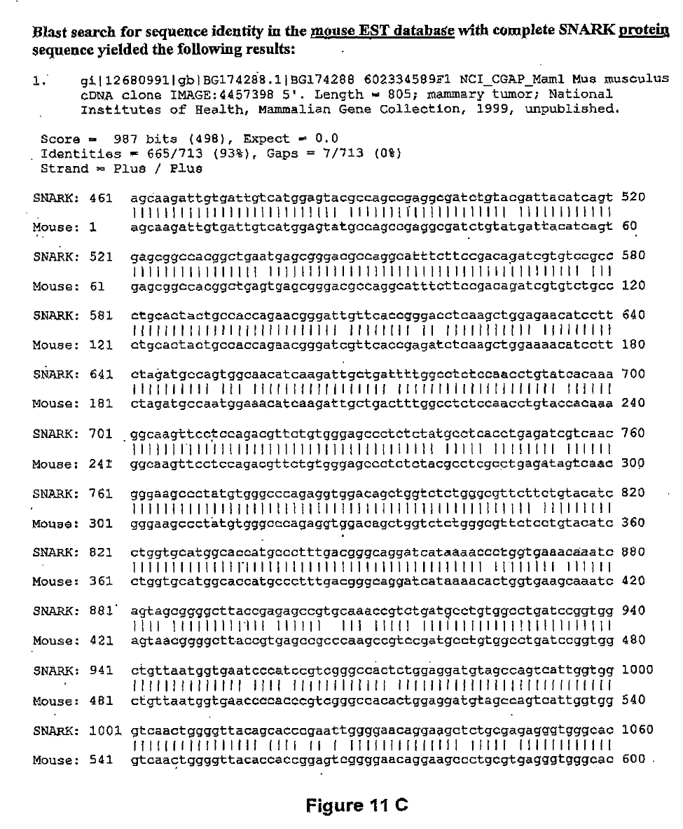

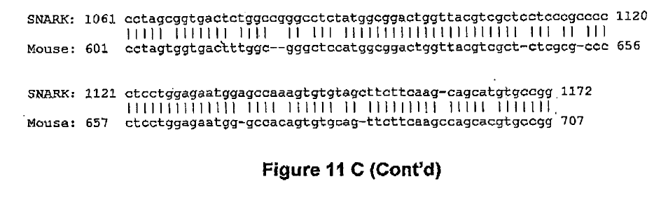

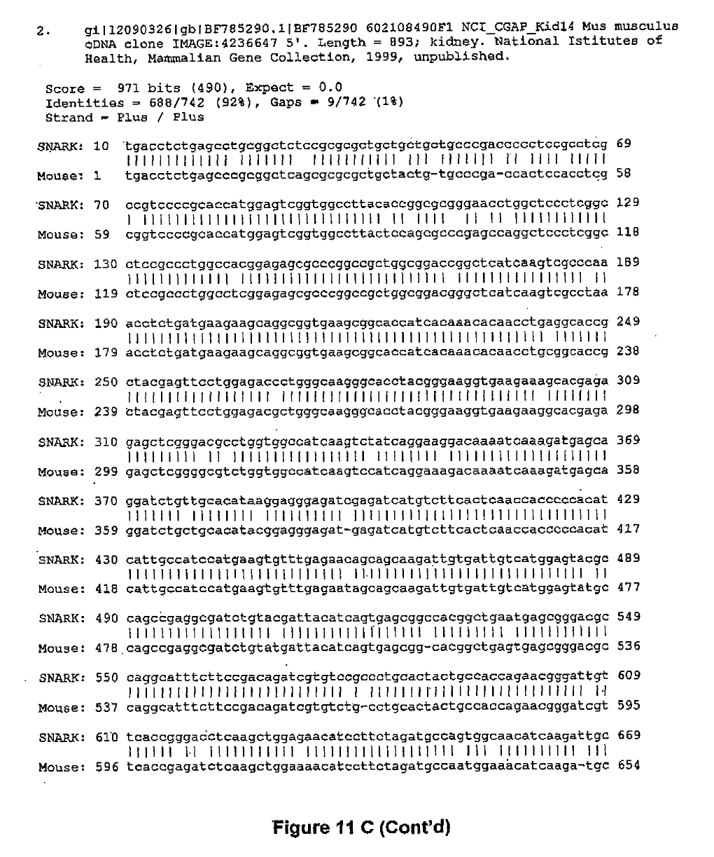

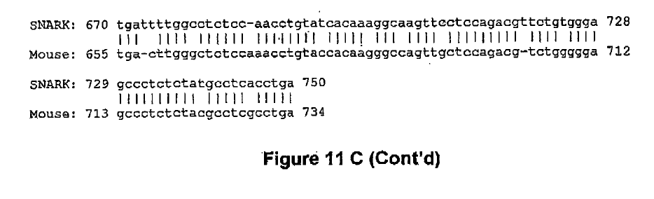

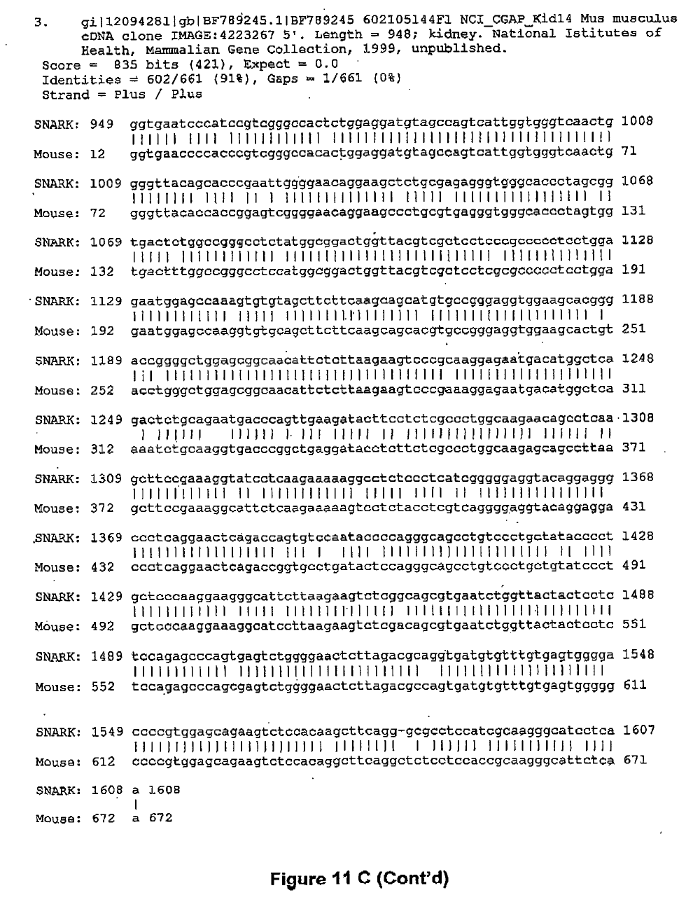

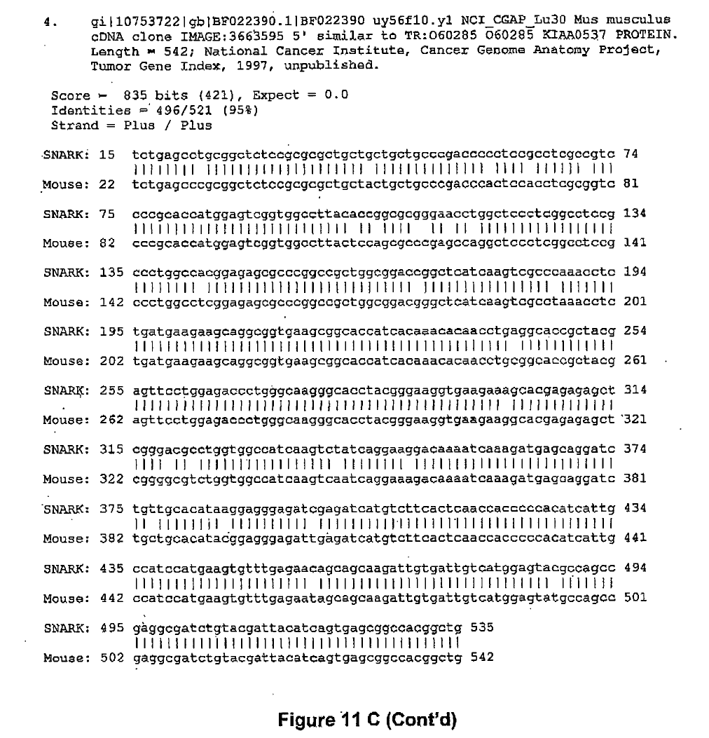

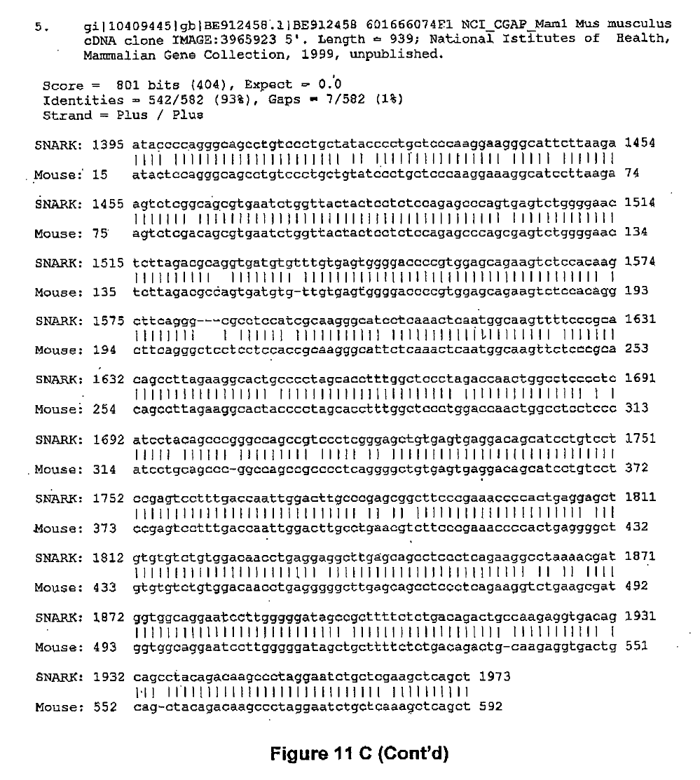

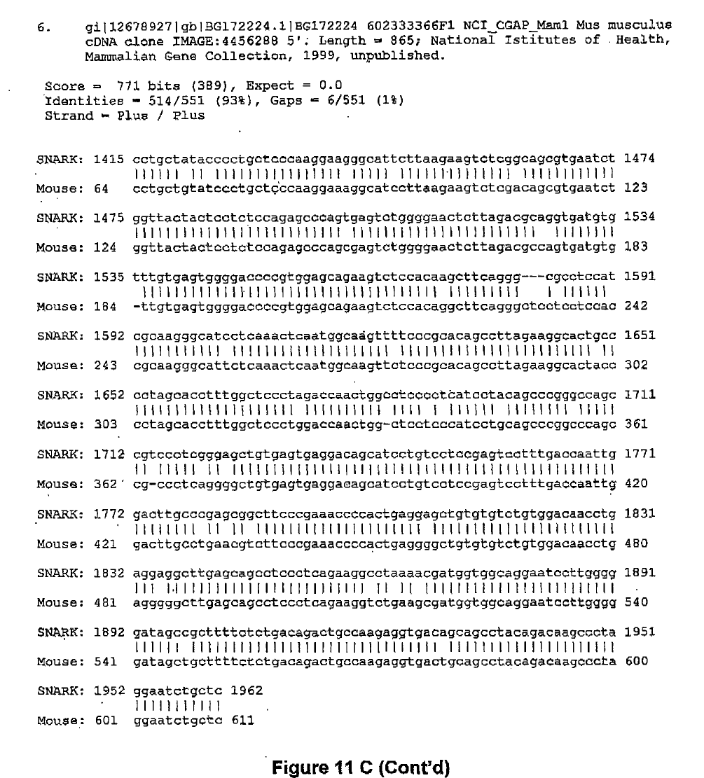

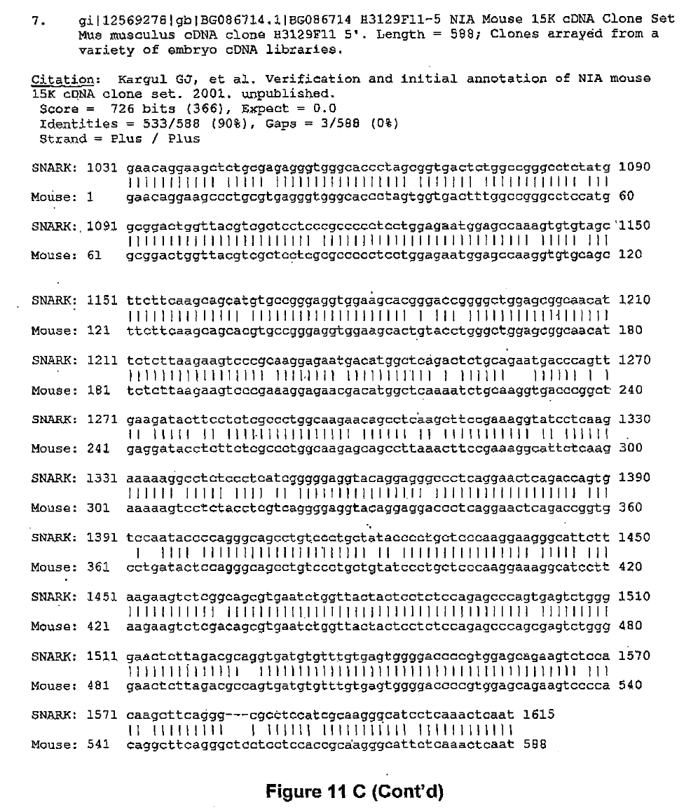

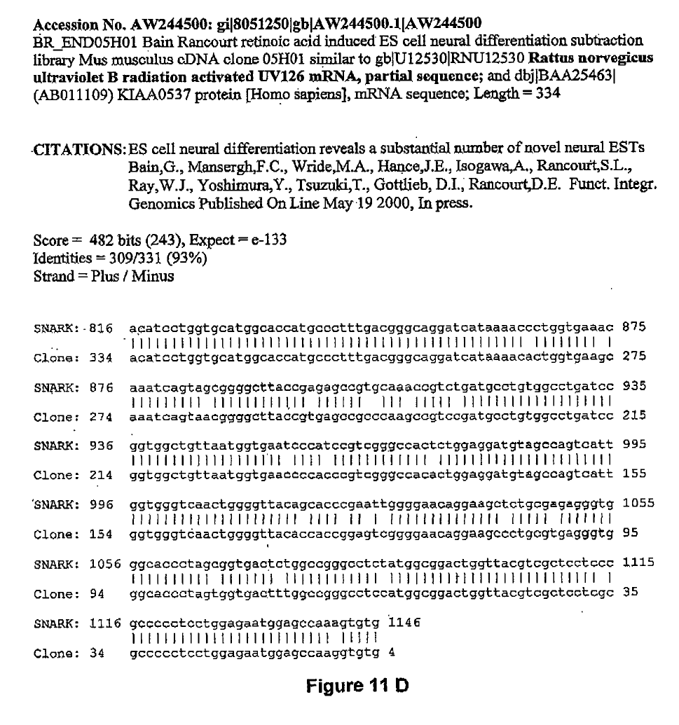

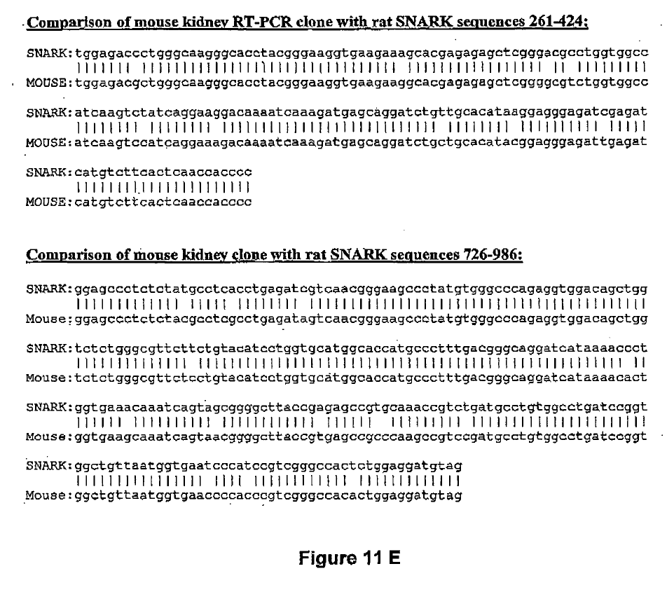

In another embodiment, the invention provides the mouse homolog of the rat SNARK of SEQ ID NO. 1. that incorporates, within its overall sequence, the 251 amino acid sequence depicted in FIG. 9B, or a variant thereof incorporating amino acid alterations that do not disrupt SNARK activity. In more specific embodiments, the mouse SNARK protein incorporates amino acid sequences that are encoded by one or more of the non-rat coding regions of the polynucleotides shown in FIGS. 11A through 11E. The mouse SNARK protein also exhibits 75%, and suitably 90% amino acid identity with rat SNARK at key functional kinase domains, as exemplified by sequence alignment shown in FIG. 9. A mouse SNARK protein also exhibits the same degree and rank order of substrate specificity as characterized for rat SNARK, as shown in Table 1, infra.

The invention also relates to chimeric versions of the SNARK protein. Such chimeric SNARK proteins are hybrid SNARK proteins in which a selected region of a given SNARK protein has been replaced, or exchanged, by a corresponding region from a SNARK homolog. Such regions suitable for exchange include, for instance, the kinase catalytic domain, or the ATP-binding region or the serine/threonine kinase active site, all of which are shown in FIG. 2 for rat SNARK. Such chimeric SNARK proteins expectedly retain SNARK activity, yet allow such function to be assessed in different SNARK backgrounds, if desired. Accordingly, in embodiments of the invention, there are provided chimeric SNARK proteins in which a functional domain or region of one form of SNARK is replaced by a corresponding region from a SNARK homolog.

The invention also embraces fragments of SNARK, including fragments of the SNARK of SEQ ID NO. 1 ,and homologous counterpart fragments of human and mouse SNARK including those shown in FIGS. 9A and 9B respectively, that are useful for various purposes. In one embodiment, the invention includes immunogenic fragments, that incorporate at least about 5, e.g., at least about 20 contiguous amino acids, and suitably up to about 200 amino acids or more corresponding to SNARK epitopes, including for instance regions of such length within the C-terminal region thereof, spanning for instance amino acids 310-630 of rat SNARK and corresponding regions of homologs such as human and mouse SNARK. Alternatively, such fragments may have an amino acid sequence that is encoded by the ESTs just described, or particularly by the human and mouse polynucleotides shown in FIGS. 10 and 11.

The invention also encompasses polynucleotides which encode SNARK or which encode SNARK variants or chimeras. Accordingly, any nucleic acid sequence which encodes the amino acid sequence of SNARK, and variants and chimerics thereof, can be used to produce recombinant molecules which express SNARK proteins. In a particular embodiment, the invention encompasses a polynucleotide consisting of a nucleic acid sequence illustrated as the SNARK-encoding region in FIG. 2, and designated herein as SEQ ID NO. 2 (i.e., nucleotides 83-1975).

Also embraced by the present invention are polynucleotides that encode human SNARK. Such polynucleotides include those partial sequences shown in FIG. 10 as having homology with the coding region of rat SNARK.

The present invention also includes polynucleotides that encode mouse SNARK. Such polynucleotides include those partial sequences shown in FIG. 11 as having homology with the coding region of rat SNARK.

It will be appreciated by those skilled in the art that as a result of the degeneracy of the genetic code, a multitude of nucleotide sequences encoding SNARK, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequence of naturally occurring SNARK.

Although nucleotide sequences which encode SNARK and its variants are preferably capable of hybridizing to the nucleotide sequence of the naturally occurring SNARK under appropriately selected conditions of stringency, it may be advantageous to produce nucleotide sequences encoding SNARK or its derivatives possessing a substantially different codon usage. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding SNARK and its derivatives without altering the encoded amino acid sequences include the production of RNA transcripts having more desirable properties, such as a greater half-life, than transcripts produced from the naturally occurring sequence.

The invention also encompasses production of DNA sequences, or fragments thereof, which encode SNARK and its fragments, entirely by synthetic chemistry. After production, the synthetic sequence may be inserted into any of the many available expression vectors and cell systems using reagents that are well known in the art. Moreover, synthetic chemistry may be used to introduce mutations into a sequence encoding SNARK or any fragment thereof.

Also encompassed by the invention are polynucleotide sequences, and especially full length SNARK-encoding sequences, that are capable of hybridizing to (1) the SNARK of SEQ ID NO. 2, or to the polynucleotides shown in FIGS. 10 and 11 as representing homologous regions within the human and mouse genes encoding SNARK, respectively, or to the complements thereof, under various conditions of stringency as taught in Wahl, G. M. and S. L. Berger (1987; Methods Enzymol. 152:399-407) and Kimmel, A. R. (1987; Methods Enzymol. 152:507-511). “Stringent conditions” or “stringency” refers to conditions that allow for the hybridization of substantially related nucleic acid sequences. For instance, such conditions will generally allow hybridization of sequence with at least about 85% sequence identity, preferably with at least about 90% sequence identity, more preferably with at least about 95% sequence identity. Polynucleotides that encode full length SNARK-encoding sequences are those which, upon expression, yield a protein having one or more SNARK activities, including autophosphorylation and response to AICAR.

The polynucleotides encoding SNARK may be extended utilizing a partial nucleotide sequence and employing various methods known in the art to detect upstream sequences such as promoters and regulatory elements. For example, one method which may be employed, “restriction-site” PCR, uses universal primers to retrieve unknown sequence adjacent to a known locus (Sarkar, G. (1993) PCR Methods Applic. 2:318-322). In particular, genomic DNA, such as the 1q32 region to which human SNARK has been mapped, is first amplified in the presence of primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region (Triglia, T. et al. (1988) Nucleic Acids Res. 16:8186). The primers may be designed using commercially available software such as OLIGO 4.06 primer analysis software (National Biosciences Inc., Plymouth, Minn.), or another appropriate program, to be 22-30 nucleotides in length, to have a GC content of 50% or more, and to anneal to the target sequence at temperatures about 68.degree.-72.degree. C. (actually between 53 C(mouse)-72 C(extension) for SNARK). The method uses several restriction enzymes to generate a suitable fragment in the known region of a gene. The fragment is then circularized by intramolecular ligation and used as a PCR template.

Another method which may be used to locate SNARK homologs is that of Parker, J. D. et al. (1991; Nucleic Acids Res. 19:3055-3060). Additionally, one may use PCR, nested primers, and PROMOTERFINDER libraries to walk genomic DNA (Clontech, Palo Alto, Calif.). This process avoids the need to screen libraries and is useful in finding intron/exon junctions. Alternatively,and as noted above, commercially available and now routine software and search engines can be used to search public databases of nucleic acid and polypeptide databases to identify homologous sequences that are likely, by closely matched sequence identities, e.g., to have SNARK activity.

When screening for full-length cDNAs for instance to find full length SNARK homologs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

The screening for further SNARK homologs can be achieved by applying standard hybridization or amplification techniques to a tissue-derived polynucleotide library. A wide variety of such libraries are commercially available. Where construction of a cDNA library is necessary, established techniques are applied. For example, isolation of a SNARK homolog typically will entail extraction of total messenger RNA from a fresh source of tissue. In this respect, it is noted that a single copy of the SNARK gene appears to be expressed in all tissues, although the testes and certain other tissues carry internally deleted forms thereof. Following conversion of message to cDNA, the library can be formed in for example a bacterial plasmid, more typically a bacteriophage. Such bacteriophage harboring fragments of the DNA are typically grown by plating on a lawn of susceptible E. coli bacteria, such that individual phage plaques or colonies can be isolated. The DNA carried by the phage colony is then typically immobilized on a nitrocellulose or nylon-based hybridization membrane, and then hybridized, under carefully controlled conditions, to a radioactively (or otherwise) labelled probe sequence to identify the particular phage colony carrying the DNA insert of particular interest, in this case a homolog of rat SNARK. The phage carrying the particular gene of interest is then purified away from all other phages from the library, in order that the foreign gene may be more easily characterized. Typically, the gene or a portion thereof is then isolated by subcloning into a plasmidic vector for convenience, especially with respect to the full determination of its DNA sequence.

The nucleotide sequences of the present invention can be engineered using methods generally known in the art in order to alter SNARK encoding sequences for a variety of reasons, including but not limited to, alterations which modify the cloning, processing, and/or expression of the gene product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, introduce mutations, and so forth.

In another embodiment of the invention, natural, modified, or recombinant nucleic acid sequences encoding SNARK may be ligated to a heterologous sequence to encode a fusion protein. For example, to screen peptide libraries for modulators, i.e., inhibitors or activators of SNARK activity, it may be useful to encode a chimeric SNARK protein that can be recognized by a commercially available antibody. A fusion protein may also be engineered to contain a cleavage site located between the SNARK encoding sequence and the heterologous protein sequence, so that SNARK may be cleaved and purified away from the heterologous moiety.

In another embodiment, sequences encoding SNARK and variant and chimeric forms thereof may be synthesized, in whole or in part, using chemical methods well known in the art (see Caruthers, M. H. et al. (1980) Nucl. Acids Res. Symp. Ser. 7:215-223; Horn, T. et al. (1980) Nucl. Acids Res. Symp. Ser. 7:225-232). Alternatively, the protein itself may be produced using chemical methods to synthesize the amino acid sequence of SNARK, or a fragment thereof. For example, peptide synthesis can be performed using various solid-phase techniques (Roberge, J. Y. et al. (1995) Science 269:202-204) and automated synthesis may be achieved, for example, using the ABI 431A peptide synthesizer (Perkin Elmer).

In another aspect of the invention, polynucleotide sequences or fragments thereof which encode SNARK, its variants, chimerics and fragments of these, may be used in recombinant DNA molecules to direct their expression in appropriate host cells. In order to express a biologically active SNARK, the nucleotide sequences encoding SNARK or a variant or chimeric thereof, may be inserted into an appropriate expression vector, i.e., a vector which contains the necessary elements for the transcription and translation of the inserted coding sequence.

Methods that are well known to those skilled in the art may be used to construct expression vectors containing sequences encoding SNARK and appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

A variety of expression vector/host systems may be utilized to contain and express sequences encoding SNARK. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems. The invention is not limited by the host cell employed.

The “control elements” or “regulatory sequences” are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, LaJolla, Calif.) or PSPORT1 plasmid (Gibco BRL) and the like may be used. The baculovirus polyhedrin promoter may be used in insect cells. Promoters or enhancers derived from the genomes of plant cells (e.g., heat shock, RUBISCO; and storage protein genes) or from plant viruses (e.g., viral promoters or leader sequences) may be cloned into the vector. In mammalian cell systems, promoters from mammalian genes or from mammalian viruses are preferable. If it is necessary to generate a cell line that contains multiple copies of the sequence encoding SNARK, vectors based on SV40 or EBV may be used with an appropriate selectable marker.

In bacterial systems, a number of expression vectors may be selected depending upon the use intended for SNARK. For example, when large quantities of SNARK are needed for the induction of antibodies, vectors that direct high level expression of fusion proteins that are readily purified may be used. Such vectors include, but are not limited to, the multifunctional E. coli cloning and expression vectors such as the BLUESCRIPT phagemid (Stratagene), in which the sequence encoding SNARK may be ligated into the vector in frame with sequences for the amino-terminal Met and the subsequent 7 residues of .beta.-galactosidase so that a hybrid protein is produced; pIN vectors (Van Heeke, G. and S. M. Schuster (1989) J. Biol. Chem. 264:5503-5509); and the like. PGEX vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

An insect system may also be used to express SNARK. For example, in one such system, Autographa californica nuclear polyhedrosis virus (AcNPV) is used as a vector to express foreign genes in Spodoptera frugiperda cells or in Trichoplusia larvae. The sequences encoding SNARK may be cloned into a non-essential region of the virus, such as the polyhedrin gene, and placed under control of the polyhedrin promoter. Successful insertion of SNARK will render the polyhedrin gene inactive and produce recombinant virus lacking coat protein. The recombinant viruses may then be used to infect, for example, S. frugiperda cells or Trichoplusia larvae in which SNARK may be expressed (Engelhard, E. K. et al. (1994) Proc. Nat. Acad. Sci. 91:3224-3227).

In mammalian host cells, a number of viral-based expression systems may be utilized. In cases where an adenovirus is used as an expression vector, sequences encoding SNARK may be ligated into an adenovirus transcription/translation complex consisting of the late promoter and tripartite leader sequence. Insertion in a non-essential E1 or E3 region of the viral genome may be used to obtain a viable virus that is capable of expressing SNARK in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Human artificial chromosomes (HACs) may also be employed to deliver larger fragments of DNA than can be contained and expressed in a plasmid. HACs of 6 to 10 M are constructed and delivered via conventional delivery methods (liposomes, polycationic amino polymers, or vesicles) for therapeutic purposes.

Specific initiation signals may also be used to achieve more efficient translation of sequences encoding SNARK. Such signals include the ATG initiation codon and adjacent sequences. In cases where sequences encoding SNARK, its initiation codon, and upstream sequences are inserted into the appropriate expression vector, no additional transcriptional or translational control signals may be needed. However, in cases where only coding sequence, or a fragment thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Different host cells which have specific cellular machinery and characteristic mechanisms for post-translational activities (e.g., BHK, CHO, HeLa, MDCK, HEK293, and W138), are available from the American Type Culture Collection (ATCC; Bethesda, Md.) and may be chosen to ensure the correct modification and processing of the foreign protein.

For long-term, high-yield production of recombinant proteins, stable expression is preferred. For example, cell lines which stably express SNARK may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1980) Cell 22:817-23) genes which can be employed in tk.sup.- or aprt.sup.-cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al (1981) J. Mol. Biol. 150:1-14) and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). Recently, the use of visible markers has gained popularity with such markers as anthocyanins, .beta. glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Alternatively, host cells that contain the nucleic acid sequence encoding SNARK and express SNARK may be identified by a variety of procedures known to those of skill in the art. These procedures include, but are not limited to, DNA-DNA or DNA-RNA hybridizations and protein bioassay or immunoassay techniques which include membrane, solution, or chip based technologies for the detection and/or quantification of nucleic acid or protein. The presence of polynucleotide sequences encoding SNARK can be detected by DNA-DNA or DNA-RNA hybridization or amplification using probes or fragments or fragments of polynucleotides encoding SNARK. Nucleic acid amplification based assays involve the use of oligonucleotides or oligomers based on the sequences encoding SNARK to detect transformants containing DNA or RNA encoding SNARK.

A variety of protocols for detecting and measuring the expression of SNARK, using either polyclonal or monoclonal antibodies specific for the protein are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on SNARK is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding SNARK include oligolabeling, nick translation, end-labeling or PCR amplification using a labeled nucleotide. Alternatively, the sequences encoding SNARK, or any fragments thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits (Pharmacia & Upjohn, (Kalamazoo, Mich.); Promega (Madison Wis.); and U.S. Biochemical Corp., Cleveland, Ohio). Suitable reporter molecules or labels, which may be used for ease of detection, include radionuclides, enzymes, fluorescent, chemiluminescent, or chromogenic agents as well as substrates, cofactors, inhibitors, magnetic particles, and the like.

Host cells transformed with nucleotide sequences encoding SNARK may be cultured under conditions suitable for the expression and recovery of the protein from cell culture. The protein produced by a transformed cell may be secreted or contained intracellularly depending on the sequence and/or the vector used. As will be understood by those of skill in the art, expression vectors containing polynucleotides which encode SNARK may be designed to contain signal sequences which direct secretion of SNARK through a prokaryotic or eukaryotic cell membrane. Other constructions may be used to join sequences encoding SNARK to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAG extension/affinity purification system (Immunex Corp., Seattle, Wash.). The inclusion of cleavable linker sequences such as those specific for Factor XA or enterokinase (Invitrogen, San Diego, Calif.) between the purification domain and SNARK may be used to facilitate purification. One such expression vector provides for expression of a fusion protein containing SNARK and a nucleic acid encoding 6 histidine residues preceding a thioredoxin or an enterokinase cleavage site. The histidine residues facilitate purification on IMIAC (immobilized metal ion affinity chromatography as described in Porath, J. et al. (1992, Prot. Exp. Purif. 3: 263-281) while the enterokinase cleavage site provides a means for purifying SNARK from the fusion protein. A discussion of vectors which contain fusion proteins is provided in Kroll, D. J. et al. (1993; DNA Cell Biol. 12:441-453).

In addition to recombinant production, fragments of SNARK may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A peptide synthesizer (Perkin Elmer). Various fragments of SNARK may be chemically synthesized separately and combined using chemical methods to produce the full length molecule.

Animal model systems which elucidate the physiological and behavioral roles of the SNARK are produced by creating transgenic animals in which the activity of SNARK is either increased or decreased, or the amino acid sequence of the expressed SNARK is altered, by a variety of techniques. Examples of these techniques include, but are not limited to: 1) Insertion of normal or mutant versions of DNA encoding SNARK, by microinjection, electroporation, retroviral transfection or other means well known to those skilled in the art, into appropriate fertilized embryos in order to produce a transgenic animal or 2) Homologous recombination of mutant or normal, human or animal versions of these genes with the native gene locus in transgenic animals to alter the regulation of expression or the structure of the SNARK sequences. The technique of homologous recombination is well known in the art. It replaces the native gene with the inserted gene and so is useful for producing an animal that cannot express native SNARK but does express, for example, an inserted mutant SNARK, which has replaced the native SNARK in the animal's genome by recombination, resulting in under expression of the transporter. Microinjection adds genes to the genome, but does not remove them, and so is useful for producing an animal that expresses endogenous and exogenous SNARK, to elicit its over-expression.

One means available for producing a transgenic animal, with a mouse as an example, is as follows: Female mice are mated, and the resulting fertilized eggs are dissected out of their oviducts. The eggs are stored in an appropriate medium such as M2 medium. DNA or cDNA encoding SNARK is cesium chloride purified from a vector by methods well known in the art. Inducible promoters may be fused with the coding region of the DNA to provide an experimental means to regulate expression of the transgene.

In another of its aspects, the present invention provides antibodies that bind to SNARK. Antibodies to SNARK may be generated using methods that are well known in the art. Such antibodies may include, but are not limited to, polyclonal, monoclonal, chimeric, single chain, Fab fragments, and fragments produced by a Fab expression library. Neutralizing antibodies, (i.e., those which inhibit dimer formation) are especially preferred for therapeutic use.

For the production of antibodies, various hosts including goats, rabbits, rats, mice, humans, and others, may be immunized by injection with SNARK or with a SNARK variant or chimeric, or any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to SNARK have an amino acid sequence consisting of at least five amino acids and more preferably at least 10 amino acids. It is also preferable that they are identical to a portion of the amino acid sequence of the natural protein, and they may contain the entire amino acid sequence of a small, naturally occurring molecule. Preferred antibodies are those raised against amino acid sequences of the SNARK protein that are unique and which do not exhibit 100% identity with the amino acid sequences of other proteins, as determined by computer-based searching of biological databases, for instance. Short stretches of SNARK amino acids may be fused with those of another protein such as keyhole limpet hemocyanin and antibody produced against the chimeric molecule. Examples of useful SNARK fragments include contiguous regions of at least 5, more desirably at least 10 amino acids and especially from 100 or about 200 amino acids within the C-terminal region of rat SNARK from residue 310 to residue 630 , or corresponding regions within SNARK homologs including human and mouse SNARK. Particularly useful fragments are those that correspond to the active site of the kinase catalytic domains I-XI, outlined in FIG. 3, as well as the ATP binding domain (amino acids 63-89) and the active site signature motif, (aa 175-187). (FIG. 2) and variants that share at least about 95% identity therewith.

Monoclonal antibodies to SNARK may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. 80:2026-2030; Cole, S. P. et al. (1984) Mol. Cell Biol. 62:109-120).

In addition, techniques developed for the production of “chimeric antibodies”, the splicing of mouse antibody genes to human antibody genes to obtain a molecule with appropriate antigen specificity and biological activity can be used (Morrison, S. L. et al. (1984) Proc. Natl. Acad. Sci. 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce SNARK-specific single chain antibodies. Antibodies with related specificity, but of distinct idiotypic composition, may be generated by chain shuffling from random combinatorial immunoglobulin libraries (Burton D. R. (1991) Proc. Natl. Acad. Sci. 88:11120-3).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. 86: 3833-3837; Winter, G. et al. (1991) Nature 349:293-299). Antibody fragments that contain specific binding sites for SNARK may also be generated. For example, such fragments include, but are not limited to, the F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed to allow rapid and easy identification of monoclonal Fab fragments with the desired specificity (Huse, W. D. et al. (1989) Science 254:1275-1281).

Various immunoassays may be used for screening to identify antibodies having the desired specificity. Numerous protocols for competitive binding or immunoradiometric assays using either polyclonal or monoclonal antibodies with established specificity are well known in the art. Such immunoassays typically involve the measurement of complex formation between SNARK and its specific antibody. A two-site, monoclonal-based immunoassay utilizing monoclonal antibodies reactive to two non-interfering SNARK epitopes is preferred, but a competitive binding assay may also be employed.

Chemical and structural homology exists among the protein kinases of the invention and the AMPK family of protein kinases. Moreover, the results herein presented indicate that SNARK activity is similar in functional terms to the activities ascribed to other members of the AMPK family. Thus, the expression of SNARK is closely associated with fuel utilization and glucose metabolism and modulation thereof will be useful to control various cellular responses to endogenous levels of glucose and other fuels. Moreover, the influence of SNARK on the levels and activation states of ATP, and the cellular cascades influenced by those levels, suggests that SNARK may also have an indirect influence on various receptor-based cascades that are driven by ATP Therefore, in diseases, disorders and conditions resulting from aberrant expression or function of SNARK, it may be desirable either to increase or decrease the availability of SNARK endogenously, either by manipulating its expression or activity levels or by manipulating the endogenous protein levels, using the techniques and agents described hereinabove. For instance, it is contemplated that upregulation of SNARK will stimulate liver CPT-1, and thereby enhance lipid metabolism in liver cells and in other cell types such as heart and skeletal muscle. Similarly, activation of SNARK in muscle cells is predicted to increase GLUT-4 and glycogen in muscle. These effects will be similar to those observed when muscle cells are treated with insulin. Hence, activation of SNARK is predicted to have insulin-like effects that would enhance the disposal of glucose into muscle, and thereby reduce plasma glucose, a desirable effect for the treatment of diabetes and some types of disorders of lipoprotein production leading to increased levels of cholesterol or triglycerides. In general, it is anticipated that SNARK will be useful to channel those effects seen to date following administration of AICAR to cells, which include increased production of GLUT-4, hexokinase and muscle glycogen (see for instance Holmes et al, Am. J. Physiol., 1999, 1990-1995 and Winder et al, J. App. Physiol., 2000, 88:2219-2226). SNARK therefore has implications for various disorders involving aberrant fuel utilization and response to metabolic or environmental stress.

It is contemplated further that SNARK will also influence the response from certain cAMP-gated receptors including ion channels, such as the cAMP-gated Chloride channels, and including the cystic fibrosis transmembrane conductance regulator (CFTR). In particular, it is contemplated that SNARK participates in this pathway, and may be useful therapeutically in the treatment of cystic fibrosis by inhibiting the hyper-functioning of the CFTR, as has been contemplated for the AMPK proteins (see Hallows, J. Clin. Invest., 2000, 105(12):1711-1721.

In one embodiment, SNARK or a variant, chimeric or fragment thereof may be administered to a subject to prevent or treat a disease associated with decreased expression of SNARK. In another embodiment, an agonist which is specific for SNARK may be administered to a subject to prevent or treat diseases including, but not limited to, those diseases listed above. In another further embodiment, a vector capable of expressing SNARK, or a fragment or a derivative thereof, may be administered to a subject to prevent or treat diseases including, but not limited to, those diseases listed above.

In a further embodiment, antagonists which decrease the expression and activity of SNARK may be administered to a subject to prevent or treat diseases predicted to be associated with increased expression of SNARK. For example disorders characterized by excess glucose utilization, increased glucose uptake, or decreased glucose production may result in hypoglycemia. In one aspect of the invention a SNARK antagonist may be administered to increase fuel production, decrease glucose uptake, and increase the levels of blood glucose in a patient suffering from hypoglycemia.

In one aspect, antibodies which specifically bind SNARK may be used directly as an antagonist or indirectly as a targeting or delivery mechanism for bringing a pharmaceutical agent to cells or tissue which express SNARK.

In another embodiment, a vector expressing the complement of the polynucleotide encoding SNARK may be administered to a subject to treat or prevent diseases including, but not limited to, those diseases listed above.

In one aspect, antibodies which specifically bind SNARK may be used directly as an antagonist or indirectly as a targeting or delivery mechanism for bringing a pharmaceutical agent to cells or tissue which express SNARK.

In a further embodiment, SNARK or a variant, chimeric or fragment thereof may be added to cells to stimulate activation of the SNARK-mediated signaling cascade, for instance to drive glucose metabolism. In particular, SNARK may be added to a cell in culture or cells in vivo using delivery mechanisms such as liposomes, viral based vectors, or electroinjection for the purpose of promoting cell proliferation and tissue or organ regeneration. Specifically, SNARK may be added to a cell, cell line, tissue or organ culture in vitro or ex vivo to stimulate cell proliferation for use in heterologous or autologous transplantation.

In an aspect of the present invention, there is provided a method for assaying SNARK activity, in which a candidate SNARK protein is incubated with a SNARK substrate under phosphorylating conditions, and then the extent of phosphorylation is measured. The candidate SNARK protein is confirmed as having SNARK activity if phosphorylation is detected in the rank order of substrate selectivity presented in Table 1 infra. Similarly, the assay can be exploited to screen and identify candidate modulators of SNARK activity, by incubating the candidate modulator with both a SNARK protein and a SNARK substrate under phosphorylating conditions, and then determining whether the candidate modulator has altered the phosphorylation relative to a control incubation from which the candidate modulator is absent. Agonists of SNARK activity are identified by an increase in phosphorylation, whereas antagonists are identified by a decrease in phosphorylation, relative to the control incubation. In this method, suitable SNARK substrates include most substrates known to be phosphorylated by the related AMPK proteins, such as the SAMS peptide identified herein. The assay can be performed against libraries of small molecules, peptides including SNARK fragments and antibodies, carbohydrates and the like.

In other embodiments SNARK activators or inhibitors can be expressed in specific cells and tissues, following which Gene Chip and Proteomics Techniques can be used to identify downstream targets in the SNARK signaling pathway that are subsequently amenable for further manipulation. Hence, the present invention further provides a method for defining one or more previously identified or novel genes and proteins that may serve as mediators, activators or inactivators of SNARK activity in cells and tissues.

In other embodiments, any of the therapeutic proteins, antagonists, antibodies, agonists, complementary sequences or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

Antagonists or inhibitors of SNARK may be produced using methods that are generally known in the art. In particular, purified SNARK may be used to produce antibodies or to screen libraries of pharmaceutical agents to identify those which specifically bind SNARK. Antagonists or inhibitors can further be identified as molecules that inhibit the phosphorylation of the SNARK protein, or as molecules that stimulate the dephosphorylation of the SNARK protein. SNARK antagonists may include SNARK variants in which the functional kinase domains, shown in FIG. 2 for rat SNARK for instance, are disrupted by site specific amino acid alteration, to generate inactive SNARK variants that compete with endogenous and functional SNARK for substrate binding. Alternatively, antagonists may be identified as molecules that bind to the SNARK protein, thereby preventing its functional activation required to exert its cellular effects. Such antagonists of SNARK activity may further include peptide fragments of SNARK that lack SNARK activity but compete with SNARK for its substrates. Such antagonist fragments may be identified for instance by deletional analysis of SNARK to truncate one or both termini, or by cleaving SNARK for instance tryptically or otherwise to generate fragments that can then be examined in the phosphorylation assay to identify antagonists, and also to identify agonists where desired.

In another embodiment of the invention, the polynucleotides encoding SNARK, or any fragment or complement thereof, may be used for therapeutic purposes. In one aspect, the complement of the polynucleotide encoding SNARK may be used in situations in which it would be desirable to block the transcription of the mRNA. In particular, cells may be transformed with sequences complementary to polynucleotides encoding SNARK. Thus, complementary molecules or fragments may be used to modulate SNARK activity, or to achieve regulation of gene function. Such technology is now well known in the art, and sense or antisense oligonucleotides or larger fragments, can be designed from various locations along the coding or control regions of sequences encoding SNARK.

Expression vectors derived from retro viruses, adenovirus, herpes or vaccinia viruses, or from various bacterial plasmids may be used for delivery of nucleotide sequences to the targeted organ, tissue or cell population. Methods that are well known to those skilled in the art can be used to construct vectors which will express nucleic acid sequence which is complementary to the polynucleotides of the gene encoding SNARK. These techniques are described both in Sambrook et al. (supra) and in Ausubel et al. (supra).

Genes encoding SNARK can be turned off by transforming a cell or tissue with expression vectors that express high levels of a polynucleotide or fragment thereof which encodes SNARK. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing complementary sequences or antisense molecules (DNA, RNA, or PNA) to the control, 5′ or regulatory regions of the gene encoding SNARK (signal sequence, promoters, enhancers, and introns). Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). The complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples which may be used include engineered hammerhead motif ribozyme molecules that can specifically and efficiently catalyze endonucleolytic cleavage of sequences encoding SNARK.

Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding SNARK. Such DNA sequences may be incorporated into a wide variety of vectors with suitable RNA polymerase promoters such as T7 or SP6. Alternatively, these cDNA constructs that synthesize complementary RNA constitutively or inducibly can be introduced into cell lines, cells, or tissues. RNA molecules may be modified to increase intracellular stability and half-life. Possible modifications include, but are not limited to, the addition of flanking sequences at the S′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections or polycationic amino polymers (Goldman, C. K. et al. (1997) Nature Biotechnology 15:462-66; incorporated herein by reference) may be achieved using methods which are well known in the art.

Any of the therapeutic methods described above may be applied to any subject in need of such therapy, including, for example, mammals such pets, poultry, livestock, primates, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may consist of SNARK, antibodies to SNARK, mimetics, agonists, antagonists, or inhibitors of SNARK. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically acceptable carriers comprising excipients and auxiliaries that facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets for instance. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art. The pharmaceutical compositions of the present invention may be manufactured in a manner that is known in the art, e.g., by means of conventional mixing, dissolving, granulating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined buffer prior to use.

For any compound, the therapeutically effective dose of SNARK-active compound can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.

A therapeutically effective dose refers to that amount of active ingredient, for example SNARK or fragments thereof, antibodies of SNARK, agonists, antagonists or inhibitors of SNARK, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

In another aspect, antibodies which specifically bind SNARK may be used for the diagnosis of conditions or diseases characterized by expression of SNARK, or in assays to monitor patients being treated with SNARK, agonists, antagonists or inhibitors. The antibodies useful for diagnostic purposes may be prepared in the same manner as those described above for therapeutics. Diagnostic assays for SNARK include methods which utilize the antibody and a label to detect SNARK in human body fluids or extracts of cells or tissues. The antibodies may be used with or without modification, and may be labeled by joining them, either covalently or non-covalently, with a reporter molecule. A wide variety of reporter molecules which are known in the art may be used, several of which are described above.

A variety of protocols including ELISA, RIA, and FACS for measuring SNARK are known in the art and provide a basis for diagnosing altered or abnormal levels of SNARK expression. Normal or standard values for SNARK expression are established by combining body fluids or cell extracts taken from normal mammalian subjects, preferably human, with antibody to SNARK under conditions suitable for complex formation The amount of standard complex formation may be quantified by various methods, but preferably by photometric, means. Quantities of SNARK expressed in control and disease, samples from biopsied tissues are compared with the standard values. Deviation between standard and subject values establishes the parameters for diagnosing disease.

In another embodiment of the invention, the polynucleotides encoding SNARK may be used for diagnostic purposes. The polynucleotides that may be used include oligonucleotide sequences, complementary RNA and DNA molecules, and PNAs. The polynucleotides may be used to detect and quantitate gene expression in biopsied tissues in which expression of SNARK may be correlated with disease. The diagnostic assay may be used to distinguish between absence, presence, and excess expression of SNARK, and to monitor regulation of SNARK levels during therapeutic intervention.

In one aspect, hybridization with PCR probes which are capable of detecting polynucleotide sequences, including genomic sequences, encoding SNARK or closely related molecules, may be used to identify nucleic acid sequences which encode SNARK. The specificity of the probe, whether it is made from a highly specific region, e.g., 10 unique nucleotides in the 5′ regulatory region, or a less specific region, e.g., especially in the 3′ coding region, or the region coding for the C-terminal 320 amino acids of SNARK, and the stringency of the hybridization or amplification (maximal, high, intermediate, or low) will determine whether the probe identifies only naturally occurring sequences encoding SNARK, alleles, or related sequences. Alternatively, and particularly for human diagnostics, the probe may have a sequence capable of revealing the presence of a polynucleotide having all or a detectable portion of any one of the human sequences depicted in FIG. 10, or of revealing the complement thereof.

Probes may also be used for the detection of related sequences, and should preferably contain at least 50% of the nucleotides from any of the SNARK encoding sequences. The hybridization probes of the subject invention may be DNA or RNA and derived from the nucleotide sequence of SEQ ID No: 2, its complement or RNA equivalents thereof, or from genomic sequence including promoter, enhancer elements, and introns of the naturally occurring SNARK. Useful such sequences are illustrated in FIG. 10 for detecting corresponding human DNA. In embodiments of the invention, such probes are suitably based on the region spanning nucleic acid residues 1-1000 of SEQ ID NO. 2. In the alternative, the probe is based on the region coding for the C-terminal 320 amino acids of rat SNARK.

Means for producing specific hybridization probes for DNAs encoding SNARK include the cloning of nucleic acid sequences encoding SNARK or SNARK derivatives into vectors for the production of mRNA probes. Such vectors are known in the art, commercially available, and may be used to synthesize RNA probes in vitro by means of the addition of the appropriate RNA polymerases and the appropriate labeled nucleotides. Hybridization probes may be labeled by a variety of reporter groups, for example, radionuclides such as 32P or 35S, or enzymatic labels, such as alkaline phosphatase coupled to the probe via avidin/biotin coupling systems, and the like.

Polynucleotide sequences encoding SNARK may be used for the diagnosis of conditions, disorders, or diseases which are associated with either increased or decreased expression of SNARK. Examples of such conditions or diseases include those associated with fuel utilization, and particularly glucose metabolism, including diabetes, as well as those associated with aberrant function of cAMP-driven channels including cystic fibrosis. The polynucleotide sequences encoding SNARK may be used in Southern or northern analysis, dot blot, or other membrane-based technologies; in PCR technologies; or in dipstick, pin, ELISA assays or microarrays utilizing fluids or tissues from patient biopsies to detect altered SNARK expression.

Such qualitative or quantitative methods are well known in the art.

In a particular aspect, the nucleotide sequences encoding SNARK and its fragments may be useful in assays that detect activation or induction of various metabolic disorders, particularly those mentioned above. The nucleotide sequences encoding SNARK may be labeled by standard methods, and added to a fluid or tissue sample from a patient under conditions suitable for the formation of hybridization complexes. After a suitable incubation period, the sample is washed and the signal is quantitated and compared with a standard value. If the amount of signal in the biopsied or extracted sample is significantly altered from that of a comparable control sample, the nucleotide sequences have hybridized with nucleotide sequences in the sample, and the presence of altered levels of nucleotide sequences encoding SNARK in the sample indicates the presence of the associated disease. Such assays may also be used to evaluate the efficacy of a particular therapeutic treatment regimen in animal studies, in clinical trials, or in monitoring the treatment of an individual patient.

In order to provide a basis for the diagnosis of disease associated with expression of SNARK, a normal or standard profile for expression is established. This may be accomplished by combining body fluids or cell extracts taken from normal subjects, either animal or human, with a sequence, or a fragment thereof, which encodes SNARK, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease.

Once disease is established and a treatment protocol is initiated, hybridization assays may be repeated on a regular basis to evaluate whether the level of expression in the patient begins to approximate that which is observed in the normal patient. The results obtained from successive assays may be used to show the efficacy of treatment over a period ranging from several days to months.

With respect to diabetes, the presence of a relatively high amount of transcript in biopsied tissue from an individual may indicate a predisposition for the development of the disease, or may provide a means for detecting the disease prior to the appearance of actual clinical symptoms. Similarly, detection of an aberrant SNARK gene, by hybridization with a SNARK-encoding polynucleotide or with a probe specific for a region suspected of carrying a mutation, can be used to identify patients with a genetic anomaly in the SNARK gene. A more definitive diagnosis of this type may allow health professionals to employ preventative measures or aggressive treatment earlier thereby preventing the development or further progression of the condition.

Additional diagnostic uses for oligonucleotides designed from the sequences encoding SNARK may involve the use of PCR. Such oligomers may be chemically synthesized, generated enzymatically, or produced in vitro. Oligomers will preferably consist of two nucleotide sequences, one with sense orientation (5′->3′) and another with antisense (3′<-5′), employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Methods which may also be used to quantitate the expression of SNARK include radiolabeling or biotinylating nucleotides, coamplification of a control nucleic acid, and standard curves onto which the experimental results are interpolated (Melby, P. C. et al. (1993) J. Immunol. Methods, 159:235-244; Duplaa, C. et al. (1993) Anal. Biochem. 212:229-236). The speed of quantitation of multiple samples may be accelerated by running the assay in an ELISA format where the oligomer of interest is presented in various dilutions and a spectrophotometric or colorimetric response gives rapid quantitation.

In further embodiments, oligonucleotides derived from any of the polynucleotide sequences described herein may be used as targets in a microarray. The microarray can be used to monitor the expression level of large numbers of genes simultaneously (to produce a transcript image), and to identify genetic variants, mutations and polymorphisms. This information may be used to determine gene function, understanding the genetic basis of disease, diagnosing disease, and in developing and in monitoring the activities of therapeutic agents.

In one embodiment, the microarray is prepared and used according to the methods described in PCT application WO95/11995 (Chee et al.), Lockhart, D. J. et al. (1996; Nat. Biotech. 14: 1675-1680) and Schena, M. et al. (1996; Proc. Natl. Acad. Sci. 93: 10614-10619), all of which are incorporated herein in their entirety by reference.

The microarray is preferably composed of a large number of unique, single-stranded nucleic acid sequences, usually either synthetic antisense oligonucleotides or fragments of cDNAs, fixed to a solid support. The oligonucleotides are preferably about 6-60 nucleotides in length, more preferably 15-30 nucleotides in length, and most preferably about 20 nucleotides in length. For a certain type of microarray, it may be preferable to use oligonucleotides that are only 7-10 nucleotides in length. The microarray may contain oligonucleotides that cover the known 5′, or 3′, sequence, or contain sequential oligonucleotides which cover the full length sequence; or unique oligonucleotides selected from particular areas along the length of the sequence. Polynucleotides used in the microarray may be oligonucleotides that are specific to a gene or genes of interest in which at least a fragment of the sequence is known or that are specific to one or more unidentified cDNAs which are common to a particular cell type, developmental or disease state. In certain situations it may be appropriate to use pairs of oligonucleotides on a microarray. The “pairs” will be identical, except for one nucleotide which preferably is located in the center of the sequence. The second oligonucleotide in the pair (mismatched by one) serves as a control. The number of oligonucleotide pairs may range from 2 to one million.

In order to produce oligonucleotides to a known sequence for a microarray, the gene of interest is examined using a computer algorithm which starts at the 5′ or more preferably at the 3′ end of the nucleotide sequence. The algorithm identifies oligomers of defined length that are unique to the gene, have a GC content within a range suitable for hybridization, and lack predicted secondary structure that may interfere with hybridization. The oligomers are synthesized at designated areas on a substrate using a light-directed chemical process. The substrate may be paper, nylon or other type of membrane, filter, chip, glass slide or any other suitable solid support.

In another aspect, the oligonucleotides may be synthesized on the surface of the substrate by using a chemical coupling procedure and an ink jet application apparatus, as described in PCT application WO95/251116 (Baldeschweiler et al.) which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array may be produced by hand or using available devices (slot blot or dot blot apparatus), materials and machines (including robotic instruments) and may contain 8, 24, 96, 384, 1536 or 6144 oligonucleotides, or any other multiple from 2 to one million which lends itself to the efficient use of commercially available instrumentation.

In order to conduct sample analysis using the microarrays, the RNA or DNA from a biological sample is made into hybridization probes. The mRNA is isolated, and cDNA is produced and used as a template to make antisense RNA (aRNA). The aRNA is amplified in the presence of fluorescent nucleotides, and labeled probes are incubated with the microarray so that the probe sequences hybridize to complementary oligonucleotides of the microarray. Incubation conditions are adjusted so that hybridization occurs with precise complementary matches or with various degrees of less complementarity. After removal of nonhybridized probes, a scanner is used to determine the levels and patterns of fluorescence. The scanned images are examined to determine degree of complementarity and the relative abundance of each oligonucleotide sequence on the microarray. The biological samples may be obtained from any bodily fluids (such as blood, urine, saliva, phlegm, gastric juices, etc.), cultured cells, biopsies, or other tissue preparations. A detection system may be used to measure the absence, presence, and amount of hybridization for all of the distinct sequences simultaneously. This data may be used for large scale correlation studies or functional analysis of the sequences, mutations, variants, or polymorphisms among samples (Heller, R. A. et al., (1997) Proc. Natl. Acad. Sci. 94:2150-55).

In another embodiment of the invention, the nucleic acid sequences that encode SNARK may also be used to generate hybridization probes which are useful for mapping the naturally occurring genomic sequence. The sequences may be mapped to a particular chromosome, to a specific region of a chromosome or to artificial chromosome constructions, such as human artificial chromosomes (HACs), yeast artificial chromosomes (YACs), bacterial artificial chromosomes (BACs), bacterial P1 constructions or single chromosome cDNA libraries as reviewed in Price, C. M. (1993) Blood Rev. 7:127-134, and Trask, B. J. (1991) Trends Genet. 7:149-154.

Fluorescent in situ hybridization (FISH as described in Verma et al. (1988) Human Chromosomes: A Manual of Basic Techniques, Pergamon Press, New York, N.Y.) may be correlated with other physical chromosome mapping techniques and genetic map data. Examples of genetic map data can be found in various scientific journals or at Online Mendelian Inheritance in Man (OMIM). Correlation between the location of the gene encoding SNARK on a physical chromosomal map and a specific disease, or predisposition to a specific disease, may help delimit the region of DNA associated with that genetic disease. The nucleotide sequences of the subject invention may be used to detect differences in gene sequences between normal, carrier, or affected individuals.

In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (Gatti, R. A. et al. (1988) Nature 336:577-580), any sequences mapping to that area may represent associated or regulatory genes for further investigation. The nucleotide sequence of the subject invention may also be used to detect differences in the chromosomal location due to translocation, inversion, etc. among normal, carrier, or affected individuals.

In another embodiment of the invention, SNARK, its catalytic or immunogenic fragments or oligopeptides thereof, can be used for screening libraries of compounds in any of a variety of drug screening techniques. The fragment employed in such screening may be free in solution, affixed to a solid support, borne on a cell surface, or located intracellularly. The formation of binding complexes, between SNARK and the agent being tested, may be measured.

Another technique for drug screening that may be used provides for high throughput screening of compounds having suitable binding affinity to the protein of interest as described in published PCT application W084/03564. In this method, as applied to SNARK large numbers of different small test compounds are synthesized on a solid substrate, such as plastic pins or some other surface. The test compounds are reacted with SNARK or fragments thereof, and washed. Bound SNARK is then detected by methods well known in the art. Purified SNARK can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding SNARK specifically compete with a test compound for binding SNARK. In this manner, the antibodies can be used to detect the presence of any peptide that shares one or more antigenic determinants with SNARK.

### EXAMPLES

### Materials and Methods

Chemicals and Materials—Radiochemicals were purchased from either ICN Biomedicals (California, USA; [³²P]-α-dATP (>3000 Ci/mmol)), Amersham Pharmacia Biotech (Baie D'Urfé, Quebec; [³²P]-γ-ATP(>5000 Ci/mmol)) or from NEN Life Sciences (Guelph, Canada; [³⁵S]-methionine (>1000 Ci/mmol)). Cell culture supplies, the Concert nucleic acid purification and Super Script preamplification systems were obtained from Canadian Life Technologies (Burlington, Ontario). The TOPO TA cloning kit and pcDNA3.1 vector were purchased from Invitrogen (San Diego, Calif.). Nylon and PVDF membranes, the T7-Sequencing kit, the GST gene fusion system and protein A Sepharose CL4B were from Amersham Pharmacia Biotech. The TNT coupled reticulocyte lysate system was from Promega (Madison, Wis.). The Bradford DC Protein assay kit was purchased from BioRad (Mississauga, Canada). The NEN Renaissance enhanced chemiluminescence (ECL) reagent plus kit and the Kodak BioMax MS and ML films were purchased from Mandel Scientific (Guelph, Ontario). The majority of chemicals and protease inhibitors were purchased from BioShop (Oakville, Ontario). Dephosphorylated myelin basic protein (MBP) was obtained from Upstate Biotechnology (New York, N.Y., U.S.A.). Dephosphorylated B-casein, whole histone and protamine sulphate were puchased from Sigma-Aldrich (Oakville, Ontario, Canada). Protein kinase C (PKC) and cAMP-dependent protein kinase (PKA) inhibitor peptides were supplied by Santa Cruz Biotechnology (Santa Cruz, Calif., U.S.A.). The SAMS peptide was synthesized by the Protein Synthesis Facility (Hospital for Sick Children, Toronto, Ontario, Canada). AMPKa2 antibody was kindly provided by Dr. Neil Ruderman (Boston, Mass., U.S.A.).

Cell Culture and Irradiation—Neonatal rat keratinocytes (NRKC) and Baby hamster kidney (BHK) cells were propagated as described previously [21,22]. Cells were seeded into 10 cm dishes at a density of 1×10⁶ NRKC cells or 3×10⁶ BHK cells and were incubated for 2-3 days prior to experimentation. The medium was removed and replaced with low-serum medium, i.e., DMEM containing 0.1% newborn calf serum (CS), 100 μg/ml streptomycin and 100 units/ml penicillin for and incubated for either 18 hours (BHK cells) or 1.5 hours (NRKC cells) prior to each experiment. Glucose deprivation was performed as previously described [10], except that 25 mM glucose was used for control plates.

DNA and RNA analysis—Sequencing of SNARK cDNAs was performed with Sp6 and T7 primers using the Sequenase T7 DNA Polymerase kit or by the York University Core Sequencing Facility (Toronto, Canada) using an Applied Biosystems Sequencer-Stretch Model and the Taq Polymerase Dye Dioxy terminator cycle sequencing method. Clustal multiple sequence alignment was performed using the MBS-Aligner program. RNA was isolated and analyzed by Northern blotting and RT-PCR as previously described [23]. The full-length SNARK CDNA was labelled with [α-P³²]-ATP by random priming technique [24] and used as a probe for Northern and Southern analyses as previously described [23].

Reverse Transcriptase-PCR—First-strand cDNA was generated using the SuperScript preamplification system and the following primers; 5′-CCGGATCCATGGAGTCGGTG-GCCTTACAC-3′ and 5′-CCGGATCCCTAAGAGTTCCCCAG-ACTCA-3′ (SNARK sequences bolded) to amplify SNARK transcripts. PCR was performed with the Perkin Elmer GeneAmp 2400 PCR system using Pfu DNA polymerase in a final volume of 50 μl. The following conditions were used: denaturation at 94° C. for 5 min, 35 cycles consisting of 94° C. for 1 min, 59° C. for 1 min and 72° C. for 2 min, and final extension at 72° C. for 10 min. Half of the PCR reaction was loaded on a 1% agarose gel and immobilized on a nylon membrane and then probed as described above. PCR products were subcloned using the TOPO TA Cloning kit and sequenced to verify identity of SNARK PCR products.

Human Chromosomal Mapping Following isolation of a partial human SNARK genomic fragment, chromosomal localization was performed by fluorescence in situ hybridization (FISH)[25] to normal human lymphocyte chromosomes counterstained with propidium iodide and 4′,6-diamidin-2-phenylindol-dihydrochloride (DAPI).

Cloning of SNARK cDNAs—The full-length clone containing the entire open reading frame of SNARK was generated using overlapping clones isolated from rat lung, kidney and keratinocytes. The complete open reading frame of rat SNARK was subcloned into the pcDNA3.1 vector.

Cloning of human SNARK was completed as follows: The human keratinocyte cell line, HaCaT, is an immortalized epithelial cell line from adult human skin that exhibits a transformed phenotype, but remains nontumorigenic (Boukamp P, et al. 1988 J Cell Biol 106:761-7). Total cellular RNA was isolated from HaCaT cells, as previously described (Chirgwin J M, et al., 1979 Biochemistry 18:5294-99) and first-strand cDNA was generated using the SuperScript preamplification system (Canadian Life Technologies; Burlington, Ontario). Reverse transcriptase-polymerase chain reaction (RT-PCR) was performed using HaCaT first-strand cDNA, a SNARK sense-strand primer (nt 240-260; 5′-tgaggcaccgctacgagttcc-3′) and anti-sense strand primer (nt 918-938; 5′-accggatcaggccacaggcat-3′) in order to amplify a 698 bp region of the SNARK transcript. RT-PCR was performed with the Perkin Elmer GeneAmp 2400 PCR system using Taq DNA polymerase (Canadian Life Technologies; Burlington, Ontario) in a final volume of 50 μl. The following conditions were used: denaturation at 94° C. for 5 min, 35 cycles consisting of 94° C. for 1 min, 59° C. for 1 min and 72° C. for 2 min, and final extension at 72° C. for 10 min. The resulting PCR reaction product was analyzed by size separation on a 1% agarose gel. The 698 bp band was excised from the agarose and the DNA was eluted using the Concert nucleic acid purification system (Canadian Life Technologies; Burlington, Ontario). PCR products were subcloned using the TOPO TA Cloning kit (Invitrogen; San Diego, Calif.) and sequencing was performed by ACGT Sequencing (Toronto, Ontario) with T7 and M13 primers. Sequence alignment and comparison of HaCaT and rat SNARK sequences was performed using the DNASIS program (Hitachi Software Engineering).

Cloning of mouse SNARK was completed as follows: Hairless mice were euthanized and kidneys were dissected and processed for RNA extraction (Chirgwin J M, et al., 1979 Biochemistry 18:5294-99). Total cellular RNA was isolated and first-strand cDNA was generated using the SuperScript preamplification system (Canadian Life Technologies; Burlington, Ontario). Reverse transcriptase-polymerase chain reaction (RT-PCR) was performed using mouse kidney first-strand cDNA, a SNARK sense-strand primer (nt 240-260; 5′-tgaggcaccgctacgagttcc-3′) and either of the anti-sense strand primers, 1) 5′-accggatcaggccacaggcat-3′(nt 918-938), or 2) 5′-ccagttgacccaccaatgactgg-3′(nt 987-1001) in order to amplify either, a 1) 698 bp, or 2) 761 bp region of the SNARK transcript. RT-PCR was performed with the Perkin Elmer GeneAmp 2400 PCR system using 2.5 Units of Pfu DNA polymerase (Stratagene, Calif., USA) in a final volume of 50 μl. The following conditions were used: denaturation at 94° C. for 5 min, 30 cycles consisting of 94° C. for 1 min, 53° C. for 1 min and 72° C. for 2 min, and final extension at 72° C. for 20 min. The resulting PCR reaction product was analyzed by size separation on a 1% agarose gel. The PCR products were excised from the agarose and the DNA was eluted using the Concert nucleic acid purification system (Canadian Life Technologies; Burlington, Ontario). PCR products were subcloned using the TOPO TA Cloning kit (Invitrogen; San Diego, Calif.) and sequencing was performed by ACGT Sequencing (Toronto, Ontario) with T7 and M13 primers. Sequence alignment and comparison of HaCaT and rat SNARK sequences was performed using the DNASIS program (Hitachi Software Engineering).

Generation of Antisera—A polypeptide containing 203 residues (corresponding to nucleotides 858-1467 of SEQ ID NO. 2) was expressed as a glutathione S-transferase (GST) fusion protein, purified by polyacrylamide electrophoresis and used to immunize three rabbits (Cocalico Biologicals, USA). Polyclonal anti-SNARK antiserum was collected and used for Western blot analysis and immunoprecipitations.

In vitro Transcription and Translation—The SNARK/pcDNA3.1 plasmid construct was used as the template for in vitro transcription and translation. The TNT coupled reticulocyte lysate system was used according to the manufacturer's protocol. Immunoprecipitation (IP) [29] of ³⁵S-SNARK and 35S-luciferase (system control) was performed with 2 μl of the TNT reactions as described below. Twenty μl of 2X SDS-loading buffer (250 mM Tris-HCl, pH 6.8, 4% (w/v) SDS, 20% (w/v) glycerol, 0.04% (w/v) bromophenol blue) was added to the complexes prior to electrophoresis on a 10% polyacrylamide/SDS gel. The gel was dried and then exposed to Kodak BioMax MS film.

Protein extracts, Immunoprecipitations and Western Analysis—Cell cultures were scraped and homogenized in lysis buffer as previously described [27]. The total protein concentration was assayed by Bradford DC protein assay. Immunoprecipitation was performed with 500 μg of total protein as previously described [10], except that the final concentration of NaCl in the IP reaction was 150 mM. Western analysis of immune complexes samples was carried out by electrophoresis on an 8% polyacrylamide/SDS gel followed by transfer and immobilization of proteins on a PVDF membrane. Membranes were blocked with 2% gelatin in Tris-buffered saline (0.5 M Tris, 1.5 M NaCl) with 0.1% Tween-20 (TBST) for 2 hours, incubated with SNARK antiserum (1:1600 dilution in TBST) for 4 hours and then incubation with a secondary antibody of horseradish peroxidase-linked anti-rabbit IgG (Amersham Pharmacia Biotech) for 1 hour. After extensive washing, the membrane was developed with ECL for 60 seconds and exposed to Kodak BioMax ML film. Equal loading of protein was verified by staining with Ponceau S.

Kinase Assay—Following immunoprecipitation with antiserum, SNARK activity was analysed by performing kinase assays with immunoprecipitated SNARK and a variety of substrates (MBP, B-casein, whole histone fraction, protamine sulphate and a synthetic peptide substrate (HMRSAMSGLHLVKRR, ‘SAMS’ peptide). Kinase assays with the SAMS peptide were performed essentially as described [28,29], except that the three washes were done with IP buffer containing 500 mM NaCl followed by a final wash in kinase assay buffer. For kinase assays performed to determine substrate specificity (with substrates other than SAMS peptide), reactions included 30 ug of substrate, 500 nM PKA inhibitor peptide and 1 uM PKC inhibitor peptide. Activities were calculated as fmol of phosphate incorporated into the SAMS peptide/min per milligram of lysate subjected to immunoprecipitation, minus the activity obtained with a blank reaction (cell lysate and Protein A-Sepharose only).

Cell Transfections—Stable SNARK-transfected cell lines were generated using the calcium phosphate-mediated method of DNA transfer as described previously using G418 to select for successful transfectants [30].

Results

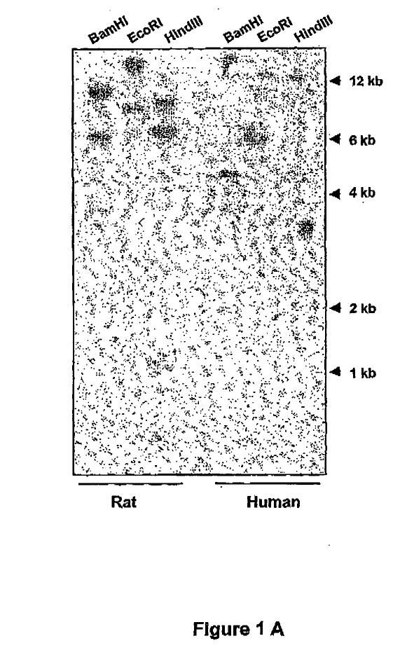

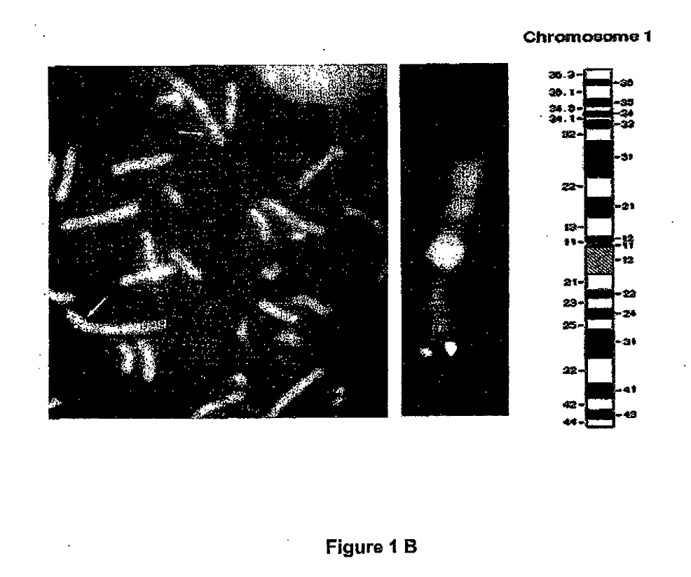

Southern blot analysis demonstrated a simple pattern of hybridizing bands in both rat and human genomic DNA samples, consistent with a single copy of SNARK in the mammalian genome (FIG. 1A). The human chromosomal localization of SNARK was examined by hybridizing an isolated rat SNARK cDNA fragment with a human P1-derived artificial chromosome (PAC) library. This experiment identified a single hybridizing genomic PAC clone that localized the human SNARK homologue to human chromosome 1q32 (FIG. 1B). Positive hybridization signals at 1q32 were noted in >90% of the metaphasic cells.

Using the original partial cDNA [20] as a probe, a full-length cDNA clone containing 2929 nucleotides was isolated with a single, uninterrupted ORF of 1893 nucleotides, beginning at nucleotide 83 and terminating at position 1975 (FIG. 2). The ORF encoded a putative protein of 630 amino acids (aa) with a predicted molecular mass of 69.95 kiloDaltons (kD) and a theoretical pI of 9.35. Comparison of the deduced amino acid sequence of the amino (N)-terminal region of the protein (aa 57-308) with other known proteins revealed 48% and 50% identity (68% similarity by including conservative substitutions scored by the BLOSUM62 matrix) within the catalytic domain of SNF1 protein kinase [31] and AMPK [32], respectively, prompting the designation of SNF1/AMPK-Related Kinase, or SNARK (FIG. 3). SNARK contains all 11 catalytic subdomains conserved in serine/threonine protein kinases [33] (FIG. 3) Analysis of the catalytic domain of SNARK using the Prosite program revealed a protein kinase ATP-binding region signature (aa 63-89) and a serine/threonine protein kinase active-site signature (aa 175-187) (FIG. 2).). The sequences at the carboxyl (C)-terminus of SNARK were distinct and not well conserved with C-terminal sequences of other SNF1/AMPK family members. The instability index was computed to be 58.40 using the Protparam Tool program, classifying SNARK as an unstable protein.

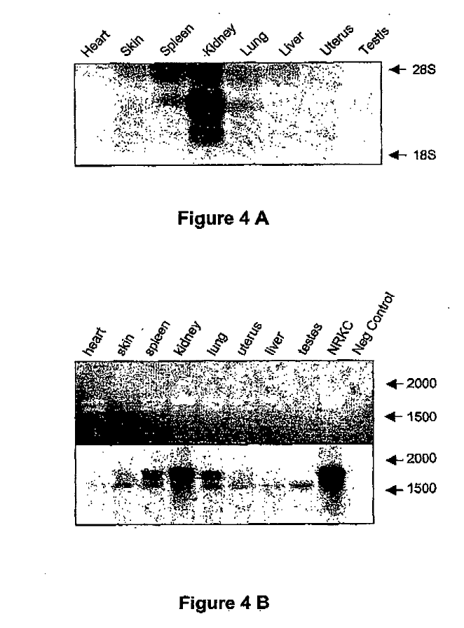

Northern analysis demonstrated SNARK RNA transcripts were most abundant in rat kidney (FIG. 4A). RT-PCR detected two SNARK CDNA products in RNA from rat heart, skin, spleen, lung, uterus, liver and a neonatal rat keratinocyte cell line (FIG. 4B). The two different SNARK RT-PCR products were cloned from several tissues, sequenced and were found to encode either authentic SNARK (1437 bp) or an internally-deleted SNARK (-Δ) transcript (1247 bp). While rat kidney contained predominantly the intact SNARK transcript and testes expressed only the 1247 bp SNARK-Δ transcript, both intact and Δ-SNARK transcripts were detected in skin, spleen, lung, uterus and liver. The SNARK-Δ transcript contained a 57 bp in-frame deletion, spanning parts of kinase domains I and II, and a 133 bp out-of-frame deletion in kinase domains IX-XI, including the invariant lysine residue involved in maximal enzyme activity. Translation of the SNARK-Δ transcript is predicted to give rise to a prematurely terminated protein of ˜415 amino acids. Internally deleted rat AMPK transcripts have also been reported [347]. The probes of the present invention, based on SNARK-encoding DNA of SEQ ID NO. 2, are thus useful to identify aberrant SNARK-encoding DNA in tissue samples, and can be used diagnostically to characterize DNA samples obtained from patents presenting with disorders related to aberrant glucose metabolism.

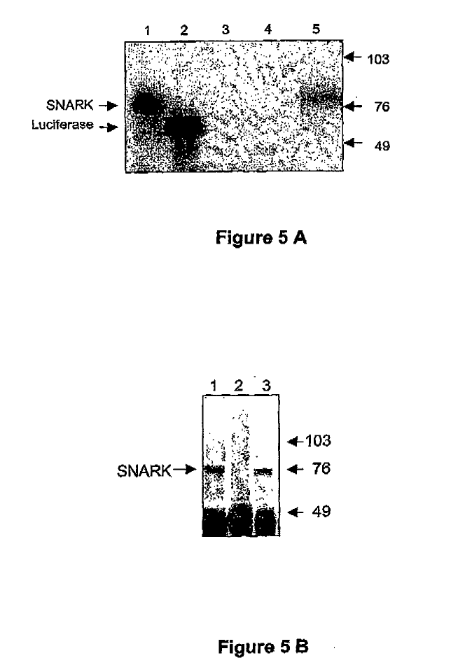

In vitro transcription and translation using the full-length SNARK cDNA template in the presence of [³⁵S]-methionine, resulted in a major protein product of ≈76 kD (FIG. 5A, lane 1) that was immunoprecipitated by SNARK antiserum (lane 5). A clearly detectable protein doublet, with a size of approximately 76-80 kD, was detected in two separate clones of SNARK-transfected BHK cells (BHK+1 and BHK+11) using SNARK antiserum (FIG. 5B; lanes 1 and 3), but not with non-immune serum (FIG. 5B; lane 2).

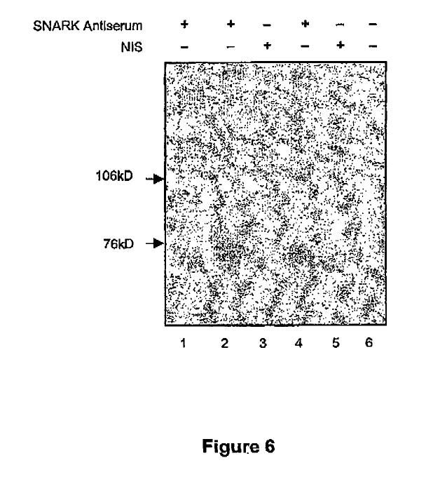

To assess whether SNARK was capable of autophosphorylation, immunoprecipitated SNARK was incubated with [³²p]-γ-ATP and reaction products were examined by SDS-Polyacrylamide gel electrophoresis (FIG. 6). Although, no autophosphorylated products were detected in samples of immunoprecipitated endogenous SNARK from wildtype BHK cells (lane 1) , one major phosphorylated band, possibly a protein doublet, was detected in the immunoprecipitates from SNARK-transfected BHK cells (FIG. 6; lanes 2 and 4). The size of the phosphorylated band(s) corresponds to the size of SNARK detected in these cell lines by Western analysis (≈76-80 kD). Furthermore, no phosphorylated proteins were observed in cell extracts following immunoprecipitation with non-immune serum (lanes 3 and 5) or samples containing extract but no antiserum (lane 6). These results indicate that SNARK is a protein kinase capable of autophosphorylation in vitro. This assay is also suitable for determining whether SNARK variants retain the autophosphorylating properties of SNARK.

To determine whether immunoprecipitated SNARK protein possessed the ability to phosphorylate protein substrates in vitro, kinase assays were performed using candidate substrates including dephosphorylated MBP, dephosphorylated β-casein, whole histone fraction, protamine sulfate, and the SAMS peptide, a well-established AMPK substrate corresponding to the site in rat acetyl-CoA carboxylase phosphorylated by AMPK [28]. Peptide inhibitors of PKA and PKC were included in these reactions to eliminate phosphorylation of these substrates by these enzymes. In the assays performed with SNARK immunoprecipitated from NRKC cell lysates, SNARK was able to phosphorylate SAMS peptide, but its ability to phosphorylate MBP, β-casein, whole histone fraction and protamine sulfate was minimal. The ability of SNARK to phosphorylate the SAMS peptide substrate was unaffected by the presence of PKA and PKC inhibitors, indicating that the observed kinase activity was not due to the phosphorylation of SAMS peptide by PKA or PKC. Results are presented in the Table below:

TABLE 1
Substrate Specificity of the SNARK protein

| | Activity |
|---|---|
| Substrate | (fmol/min per mg) |
| SAMS peptide | 145 ± 46* |
| MBP | 91 ± 11 |
| B-casein | 37 ± 12 |
| Whole histone fraction | 19 ± 6 |
| Protamine sulfate | 7 ± 2 |

Immunoprecipitation of 500 ug of NRKC cell lysate with SNARK antiserum was performed as described above. Phosphotransferase activity was assayed with either SAMS peptide (250 uM) or 30 ug of dephosphorylated MBP, dephosphorylated β-casein, whole histone fraction or protamine sulfate in the presence of 1 μM PKC inhibitor and 500 nM PKA inhibitor peptides. Phosphortransferase activity is expressed in fmol of phosphate transferred to the SAMS peptide/min at 30 C per mg of protein subjected to immunoprecipitation. Results are means±S.E.M. for two individual assays with at least eight samples per group. *p<0.01 compared with all other substrates.

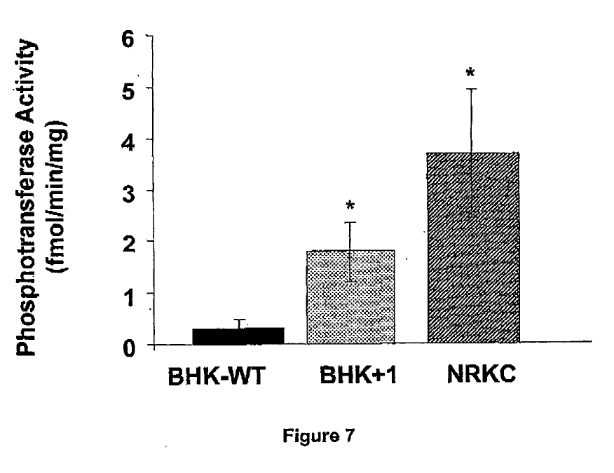

Kinase assays performed with the SAMS peptide on immunoprecipitated SNARK from wildtype BHK cells gave a low basal level of SAMS phosphotransferase activity (FIG. 7A, solid box). In contrast, the basal SNARK phosphotransferase activity detected in SNARK-transfected BHK cells (BHK+1) and in rat NRKC cells was 3.4-fold and 2-fold higher, respectively, than the levels found in wildtype BHK cells (FIG. 7, solid boxes), Since AMPK-α2 antibodies immunoprecipitate 4-fold less SAMS phosphotransferase activity than the SNARK antiserum in SNARK-transfected BHK cells (data not shown), the phosphotransferase activity detected in this assay system appears to be specific for SNARK kinase activity. The SNARK antiserum does not cross-react with AMPK isoforms in rat skeletal muscle and AMPK-α2 antibodies do not immunoprecipitate any detectable SAMS phosphotransferase activity in SNARK-transfected BHK cells, these findings indicate that SNARK exhibits AMPK-like kinase activity.

The kinase assay just described is useful to identify functional variants of SNARK, and chimeric forms of SNARK, that retain its phosphorylation properties. The kinase assay is also useful to identify such variants and chimerics of SNARK that retain its substrate activity in hierarchal terms relative to the substrates tested. That is, it is expected that all SNARK proteins, whether wildtype (such as rat SNARK and its mammalian homologs), variant, or chimeric, will exhibit the rank order of phosphorylating activity shown in Table 1 above with respect to those kinase substrates.

AMPK is activated by environmental stresses that lead to depletion of cellular ATP and elevation of AMP [1]. To evaluate the effects of cellular stress on SNARK activity, there was examined the effects of AMP on SNARK phosphotransferase activity in wildtype BHK, SNARK-transfected BHK and NRKC cell lines. Although no significant change in SNARK phosphotransferase activity was observed when wildtype and SNARK-transfected cell lysates were assayed in the presence of 200 μM AMP, SNARK phosphotransferase activity increased by 1.7 fold (p<0.001) in NRKC cells (FIG. 7, hatched box).

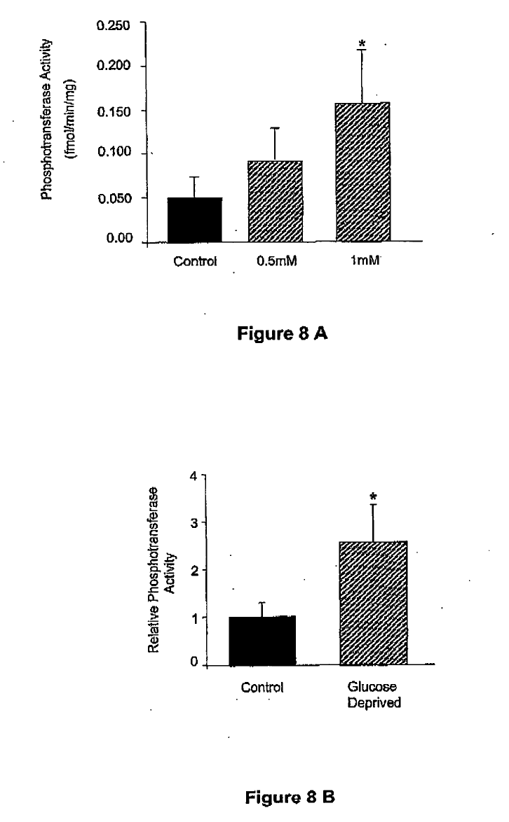

The adenosine analogue, 5-aminoimidizole-4-carboxamide riboside (AICAR), provides a means of stimulating AMPK activity in whole cells, even in the presence of high glucose concentrations, and mimics the effects of AMP on the AMPK cascade [35,36]. Intriguingly, SNARK activity was induced 2.8-fold (p<0.05) in NRKC cells when treated with 1 mM AICAR for one hour (FIG. 8A, hatched box), as compared to SNARK activity measured in untreated NRKC cells (solid box). Since AICAR is taken up into cells and converted by adenosine kinase into a phosphorylated monophosphate form (ZMP) which mimics the effects of AMP on both the allosteric activation and the phosphorylation of AMPK via AMPK kinase, this data implies that SNARK can be covalently modified by AMPK kinase in response to elevated cellular AMP.

The concentration of glucose in culture medium is an important modulator of both SNF1 activity in yeast cells [37, 38] and AMPK activity in pancreatic β-cells [10]. SNARK activity assayed in wildtype BHK cells deprived of glucose for 90 minutes was 2.6-fold higher (p<0.03; FIG. 8B, hatched box) than activity levels measured in BHK cells cultured in 25 mM glucose (solid box). This result suggests that SNARK activity responds to glucose deprivation in a manner similar to yeast SNF1 and rat AMPK [10, 38]. Furthermore, immunoreactive SNARK was localized to the exocrine and endocrine compartments of the human pancreas. Consistent with this finding was the Western analysis of cell lysates from the rat INS-1 (insulinoma), mouse αTC (glucagonoma) and hamster InR1G9 (glucagonoma) cell lines which revealed SNARK immunoreactive proteins in the INS-1 and αTC cell lines corresponding in size to that found in SNARK-transfected BHK cells (i.e., approx 80 kDa). Although no 80 kDa SNARK protein was detected in the hamster cell line InR1G9, a protein migrating in approximately 106 kDa was detected in both the InR1G9 and SNARK-transfected BHK cells. This larger protein might represent a form of SNARK that undergoes differential post-translational modification.

This study describes the cloning of a member of the SNF1/AMPK family of serine/threonine kinases localized to human chromosome 1q32. The 3.5 kb SNARK mRNA encodes for a 76-80 kDa protein containing amino acid motifs characteristic of serine/threonine kinases. SNARK mRNA transcripts were detectable by RT-PCR in almost all tissues examined, hence like AMPK [28, 34, SNARK is not a cell-specific kinase. The detection of two SNARK RNA isoforms, including the SNARK (-Δ) transcript that is predicted to give rise to a non-kinase protein, highlights the importance of using probes or primers specific for detection of full-length SNARK in future studies of SNARK expression and localization in cell types. Similarly, both AMPK and SNARK are able to phosphorylate the SAMS peptide substrate, derived from the site on acetyl Co-A carboxylase that is specifically phosphorylated by AMPK. Importantly, the antisera used in our studies for analysis of SNARK autophosphorylation and kinase activity was directed against peptide sequences in the carboxy-terminal region of SNARK that exhibit no homology to AMPK. Taken together, the structural and functional data establish SNARK as a new mammalian member of the SNF1/AMPK family of kinases.

Within its catalytic domain, SNARK is most closely related to the SNF1/AMPK family of protein kinases, possessing a high degree of homology at the amino acid level. Members of the SNF1/AMPK protein kinase family have been highly conserved throughout evolution and the hallmark members of this family, SNF1 and AMPK, are generally thought to represent key metabolic sensors in stress response systems, although each responds to different types of stresses. AMPK is activated by environmental and cellular stresses [1], including exercise and glucose deprivation [10,39]. These stresses deplete cellular ATP and, via the adenylate kinase reaction, elevate AMP which serves as a switch to activate AMPK activity. The finding that SNARK activity can be stimulated by exposure to both AMP and AICAR suggests that SNARK, like AMPK signaling cascade, is sensitive to levels of cellular AMP.

Although UVB is a constant source of cellular stress for the skin cell since it is a major component of terrestrial sunlight, the molecular signaling mechanisms induced by UVB are incompletely understood. It has been reported that UVB significantly activates c-Jun NH₂-terminal kinases (JNKs) in keratinocytes and induces translocation of membrane-associated protein kinase C isoforms from cytosol to membrane in epidermal cells mediating signal transduction and apoptosis through activation of extracellular-regulated kinases (Erks) and JNKs. In addition, the FKBP/FRAP/p70S6K signaling cascade has been identified as a pathway regulated by UVB-induced DNA damage and repair. Furthermore, UVB-induces growth inhibition of keratinocytes in hyperproliferative skin disorders via downregulation of the type II interleukin-8 receptor (CXCR-2). These and other studies support the contention that the UVB signal is transduced via both membrane-associated and cytosolic signal pathways to the nucleus, resulting in multiple cutaneous effects. It is conceivable that these responses to UVB radiation may lead to ATP:AMP ratio perturbations which activate SNARK in keratinocytes in an attempt to re-establish metabolic equilibrium within the cell.

As noted, SNARK activity can be regulated by the concentration of glucose in the medium. This is consistent with recent experiments demonstrating that AMPK in pancreatic β-cells is modulated in response to the extracellular glucose concentration [10]. Glucose deprivation of pancreatic β cell lines resulted in a >5-fold activation of AMPK activity within 30 minutes of glucose removal [10]. AMPK activation was associated with a large increase in the cellular AMP/ATP ratio resulting from the low levels of extracellular glucose and correlated inversely with insulin secretion. Conversely, AMPK activity was inhibited by increasing glucose concentrations in MIN6 beta cells and immunoneutralization of the AMPK complex diminished glucose-regulated gene transcription in vitro [40]. Because SNARK immunoreactivity is also localized to human islets and rodent islet cell lines, it is likely that SNARK is modulator of islet cell response to metabolic stress, such as hypoglycemia.

The citations referenced herein are incorporated herein by reference, and are listed below in full:

1. Hardie et al, 1998, Ann. Rev. Biochem., 67:821.

2. Carlson, 1999, Curr. Opinion Microbiol., 2:202

3. Alderson et al, 1991, Proc. Nati. Acad. Sci. USA, 88:8602

4. Le Guen et al, 1992, Gene, 120:249

5. Bouly et al, 1999, Plant J., 18:541

6. Hannappel et al, 1995, Plant Mol. Biol., 27:1235

7. Halford et al, 1992, Plant J., 2:791

8. Muranaka et al, 1994, Mol. Cell. Biol., 14:2958

9. Takano et al, 1998, Mol. Gen. Genet., 260:388

10. Salt et al, 1998, Biochem. J., 335:533

11. Sugden et al, 1999, Plant Physiol., 120:257

12. Bracchi et al, 1996, Mol. Biochem. Parasitol., 76:299

13. Davies et al, 1999, Plant Cell, 111: 1179

14. Wang et al, 1999, FEBS Lett., 453:135

15. Becker et al, 1996, Eur. J. Biochem., 235:736

16. Heyer et al, 1997, Mol. Reprod. Dev., 47:148

17. Ruiz et al, 1994, Mech. Dev., 48:153

18. Inglis et al, 1993, Mamm. Genome, 4:401

19. Gardner et al, 2000, Genomics, 63:46

20. Rosen et al, 1995, Am. J. Physiol., 268:C846

21. Rosen et al, 1990, Cancer Res., 50:2631

22. Yusta et al, 1999, J. Biol. Chem., 274:30459

23. Shahmolky et al, 1999, Photochem Photobiol., 70:341

24. Feinberg & Vogelstein, 1983, Anal. Biochem., 132:6

25. Lichter et al, 1990, Science, 247:64

26. Ip & Davis, 1998, Curr. Opin. Cell Biol., 10:205

27. Salt et al, 1998, Biochem. J., 334:177

28. Davies et al, 1989, Eur. J. Biochem., 186:123

29. Dale et al, 1995, FEBS Lett., 361:191

30. Chen & Okayama, 1988, BioTechniques, 6:632

31. Celenza & Carson, 1986, Science, 233:1175

32. Carling et al, 1994, J. Biol. Chem., 269:11442

33. Hanks & Hunter, 1995, FASEB J., 9:576

34. Gao et al, 1995, Biochim. Biophys. Acta., 1266:73

35. Corton et al, 1995, Eur. J. Biochem., 229:19509

36. Henin et al, 1996, Biochim. Biophys. Acta., 1290:197

37. Woods et al, 1994, J. Biol. Chem., 269:19509

38. Wilson et al, 1996, Curr. Biol., 6:1426

39. Winder & Hardie, 1996, Am. J. Physiol., 270:E299

40. da Silva et al, 2000, Proc. Natl. Acad. Sci. USA, 97:4023

We claim:

1. An isolated SNARK protein, selected from among the group consisting of:
(1) the rat SNARK protein of SEQ ID NO. 1
(2) a mammalian homolog of (1)
(3) a variant of (1) or (2), and
(4) a chimeric form of (1) or (2) in which a SNARK domain is exchanged with a heterologous SNARK domain,
wherein said variant and said chimeric forms of SNARK retain SNARK activity and have at least 70% amino acid identity with (1) or (2).

2. An isolated SNARK protein according to claim 1, which is the rat SNARK protein of SEQ ID. NO. 1.

3. An isolated SNARK protein according to claim 1, which is the human homolog of the rat SNARK protein of SEQ ID NO. 1.

4. An isolated SNARK protein according to claim 1, which is the murine homolog of the rat SNARK protein of SEQ ID NO. 1.

5. An isolated SNARK protein according to any one of claims 1-4, in detectably labeled form.

6. An immunogenic fragment of SNARK protein defined in any one of claims 1-4.

7. A detectably labeled fragment of a SNARK protein defined in any one of claims 1-4.

8. An isolated polynucleotide that encodes a SNARK protein defined in any one of claims 1-4.

9. A detectably labeled polynucleotide that hybridizes with a polynucleotide according to claim 8 or with the complement thereof.

10. A vector incorporating a polynucleotide as defined in claim 8.

11. A vector according to claim 10, wherein said vector further incorporates expression controlling elements linked operably with said polynucleotide to drive expression thereof in a host cell.

12. A host cell incorporating a vector according to claim 11.

13. A method for producing a SNARK protein, comprising the step of culturing a host cell as defined in claim 12.

14. An antibody which binds selectively to a SNARK protein according to any one of claims 1-4.

15. A detectably labeled antibody which binds selectively to a SNARK protein according to any one of claims 1-4.

16. A method for identifying a SNARK activity modulator, comprising the step of incubating a candidate SNARK modulator with a SNARK protein according to any one of claims 1-4 and with a SNARK substrate under phosphorylating conditions, and then determining whether phosphorylation has been modulated relative to a control incubation in which no candidate SNARK modulator has been present.

17. A method for identifying a SNARK expression modulator, comprising the step of incubation a SNARK-producing cell under conditions mediating SNARK expression with a candidate modulator of SNARK expression, and then determining whether SNARK gene expression or SNARK protein production has occurred in the presence of said SNARK expression modulator, relative to a control incubation in which no candidate SNARK expression modulator has been present.

18. A modulator of SNARK activity, whenever identified in accordance with the method according to claim 16.

19. A modulator of SNARK gene expression, whenever identified in accordance with the method according to claim 17.

20. A method for modulating SNARK activity in a cell, the method comprising the step of delivering to the cell a SNARK activity modulator identified by the method according to claim 16.

21. A composition comprising a SNARK protein as defined in any one of claims 1-4, and a carrier suitable for delivering the SNARK protein to a mammal.

22. A composition comprising a SNARK antibody, and a carrier suitable for delivering the SNARK antibody to a mammal.

23. A method for inhibiting the activity of a SNARK protein as defined in any one of claims 2-4, comprising the step of delivering to a cell expressing said protein, an agent selected from (1) an oligonucleotide or polynucleotide that hybridizes with the endogenous polynucleotide encoding said SNARK protein to arrest the transcription or translation thereof, and (2) a polypeptide that binds to or competes with said protein to inhibit the phosphorylating activity thereof.

24. A method for enhancing the activity of a SNARK protein as defined in any one of claims 2-4, comprising the step of delivering to a cell expressing said protein, an agent selected from (1) an expressible gene encoding said protein thereby to enhance the level of said protein in said cell, and (2) an amount of said protein effective to increase the presence of said protein in said cell thereby to enhance the protein activity.

25. A method for identifying downstream targets in the SNARK signaling pathway, comprising delivering a SNARK protein, a gene coding for therefor, or an activator of the expression of said gene to a cell or tissue or organism, and then comparing the effect of said administration on the proteomic or genomic composition of said cell, tissue or organism, relative to an untreated counterpart thereof, thereby to identifying downstream targets in the SNARK signaling pathway as proteins or genes that are modulated by said administration.

26. A substrate having immobilized thereon a chemical entity selected from (1) a SNARK protein as defined in claims 1-4, (2) a fragment thereof, (3) an antibody selective therefor, (4) a gene coding therefor, or (5) a fragment of said gene.


SEQUENCE LISTING (109 sequences)

SEQ ID NO: 1 - DNA, 2929 bp, RAT
ggcacgaggtgacctctgagcctgcggctctccgcgcgctgctgctgctgcccgaccccctccgcctcgccgtccccgcaccatggagtcggtggccttacaccggcgcgggaacctggctccctcggcctccgccctggccacggagagcgcccggccgctggcggaccggctcatcaagtcgcccaaacctctgatgaagaagcaggcggtgaagcggcaccatcacaaacacaacctgaggcaccgctacgagttcctggagaccctgggcaagggcacctacgggaaggtgaagaaagcacgagagagctcgggacgcctggtggccatcaagtctatcaggaaggacaaaatcaaagatgagcaggatctgttgcacataaggagggagatcgagatcatgtcttcactcaaccacccccacatcattgccatccatgaagtgtttgagaacagcagcaagattgtgattgtcatggagtacgccagccgaggcgatctgtacgattacatcagtgagcggccacggctgaatgagcgggacgccaggcatttcttccgacagatcgtgtccgccctgcactactgccaccagaacgggattgttcaccgggacctcaagctggagaacatccttctagatgccagtggcaacatcaagattgctgattttggcctctccaacctgtatcacaaaggcaagttcctccagacgttctgtgggagccctctctatgcctcacctgagatcgtcaacgggaagccctatgtgggcccagaggtggacagctggtctctgggcgttcttctgtacatcctggtgcatggcaccatgccctttgacgggcaggatcataaaaccctggtgaaacaaatcagtagcggggcttaccgagagccgtgcaaaccgtctgatgcctgtggcctgatccggtggctgttaatggtgaatcccatccgtcgggccactctggaggatgtagccagtcattggtgggtcaactggggttacagcacccgaattggggaacaggaagctctgcgagagggtgggcaccctagcggtgactctggccgggcctctatggcggactggttacgtcgctcctcccgccccctcctggagaatggagccaaagtgtgtagcttcttcaagcagcatgtgccgggaggtggaagcacgggaccggggctggagcggcaacattctcttaagaagtcccgcaaggagaatgacatggctcagactctgcagaatgacccagttgaagatacttcctctcgccctggcaagaacagcctcaagcttccgaaaggtatcctcaagaaaaaggcctctccctcatcgggggaggtacaggagggccctcaggaactcagaccagtgtccaataccccagggcagcctgtccctgctatacccctgctcccaaggaagggcattcttaagaagtctcggcagcgtgaatctggttactactcctctccagagcccagtgagtctggggaactcttagacgcaggtgatgtgtttgtgagtggggaccccgtggagcagaagtctccacaagcttcagggcgcctccatcgcaagggcatcctcaaactcaatggcaagttttcccgcacagccttagaaggcactgcccctagcacctttggctccctagaccaactggcctcccctcatcctacagcccgggccagccgtccctcgggagctgtgagtgaggacagcatcctgtcctccgagtcctttgaccaattggacttgcccgagcggcttcccgaaaccccactgaggagctgtgtgtctgtggacaacctgaggaggcttgagcagcctccctcagaaggcctaaaacgatggtggcaggaatccttgggggatagccgcttttctctgacagactgccaagaggtgacagcagcctacagacaagccctaggaatctgctcgaagctcagctgaggaggagaggcagtgccccagtgatggggtagactcttagaggggtttgcagaggaacctgggtagattccccagggttgtagagtacatcaagaactctctctctgtcttcagcctgattgaacctggaggctgagagaaatagcagagatatggaaaggactgacctacagagtctgactgcagatgtgagcggcacagagactgaaagtgcctacctcctttatgctgagtgctacccatggcatctcccccctgctctctgccagtgtcagggtgtacccacataagttcctgttcgcatcgaccaccagggttagaaccctgacatccctggaagtaatgtggagcaacctcgcttatttaaagaggaaacagcctctggtttccatctctgctgctgtgcatctcaaagacctagaaagactcaactgctgtttcacttcatctcaaggggacctcagagacctgagccttgaagctgttcctgataaccagactatgatggatatgtctgtttctcaggccagcaggacccagaatgtgctgacttatttatttttgtgattctcacttctggtttctgtttcgtttttttgttgttggttttgttgttgtcgtttgtttcttgttttttgtttgtttgtttgtttgtttgtttgttttttaaagtgaattttgctgctttcggtaatgtgaatgctgtgttctggggaaagccactgtgtcattgaagtgtgtgtacagagaagtatttggcagtgattccttctaatggggggtggccttttcagatgtatgtcttgagcactgtctggattgggtctcctgtcccctcacaccagaggctgtccaccctccctcatctgtggccaaaaaaaacctcattaaaaccagcaacggcaactggaaaaaaaaaaattttttttttttttttttttttttttttttttt

SEQ ID NO: 2 - DNA, 177 bp, RAT
gcccggccgctggcggaccggctcatcaagtcgcccaaacctctgatgaagaagcaggcggtgaagcggcaccatcacaaacacaacctgaggcaccgctacgagttcctggagaccctgggcaagggcacctacgggaaggtgaagaaagcacgagagagctcgggacgcctggtg

SEQ ID NO: 3 - DNA, 122 bp, RAT
ggtggccatcaagtctatcaggaaggacaaaatcaaagatgagcaggatctgttgcacataaggagggagatcgagatcatgtcttcactcaaccacccccacatcattgccatccatgaag

SEQ ID NO: 4 - DNA, 150 bp, RAT
agtgtttgagaacagcagcaagattgtgattgtcatggagtacgccagccgaggcgatctgtacgattacatcagtgagcggccacggctgaatgagcgggacgccaggcatttcttccgacagatcgtgtccgccctgcactactgcca

SEQ ID NO: 5 - DNA, 109 bp, RAT
agattgctgattttggcctctccaacctgtatcacaaaggcaagttcctccagacgttctgtgggagccctctctatgcctcacctgagatcgtcaacgggaagcccta

SEQ ID NO: 6 - DNA, 11 bp, RAT
tgtgggcccag

SEQ ID NO: 7 - DNA, 121 bp, RAT
aggtggacagctggtctctgggcgttcttctgtacatcctggtgcatggcaccatgccctttgacgggcaggatcataaaaccctggtgaaacaaatcagtagcggggcttaccgagagcc

SEQ ID NO: 8 - DNA, 13 bp, RAT
gtgcaaaccgtct

SEQ ID NO: 9 - DNA, 421 bp, RAT
gatgcctgtggcctgatccggtggctgttaatggtgaatcccatccgtcgggccactctggaggatgtagccagtcattggtgggtcaactggggttacagcacccgaattggggaacaggaagctctgcgagagggtgggcaccctagcggtgactctggccgggcctctatggcggactggttacgtcgctcctcccgccccctcctggagaatggagccaaagtgtgtagcttcttcaagcagcatgtgccgggaggtggaagcacgggaccggggctggagcggcaacattctcttaagaagtcccgcaaggagaatgacatggctcagactctgcagaatgacccagttgaagatacttcctctcgccctggcaagaacagcctcaagcttccgaaaggtatcctcaagaaaaagg

SEQ ID NO: 10 - DNA, 439 bp, RAT
cccctgctcccaaggaagggcattcttaagaagtctcggcagcgtgaatctggttactactcctctccagagcccagtgagtctggggaactcttagacgcaggtgatgtgtttgtgagtggggaccccgtggagcagaagtctccacaagcttcagggcgcctccatcgcaagggcatcctcaaactcaatggcaagttttcccgcacagccttagaaggcactgcccctagcacctttggctccctagaccaactggcctcccctcatcctacagcccgggccagccgtccctcgggagctgtgagtgaggacagcatcctgtcctccgagtcctttgaccaattggacttgcccgagcggcttcccgaaaccccactgaggagctgtgtgtctgtggacaacctgaggaggcttgagcagcctccctcagaaggcc

SEQ ID NO: 11 - DNA, 47 bp, RAT
ttgggggatagccgcttttctctgacagactgccaagaggtgacagc

SEQ ID NO: 12 - DNA, 56 bp, RAT
aaagtgaattttgctgctttcggtaatgtgaatgctgtgttctggggaaagccact

SEQ ID NO: 13 - DNA, 1186 bp, RAT
gcccggccgctggcggaccggctcatcaagtcgcccaaacctctgatgaagaagcaggcggtgaagcggcaccatcacaaacacaacctgaggcaccgctacgagttcctggagaccctgggcaagggcacctacgggaaggtgaagaaagcacgagagagctcgggacgcctggtggccatcaagtctatcaggaaggacaaaatcaaagatgagcaggatctgttgcacataaggagggagatcgagatcatgtcttcactcaaccacccccacatcattgccatccatgaagtgtttgagaacagcagcaagattgtgattgtcatggagtacgccagccgaggcgatctgtacgattacatcagtgagcggccacggctgaatgagcgggacgccaggcatttcttccgacagatcgtgtccgccctgcactactgccaccagaacgggattgttcaccgggacctcaagctggagaacatccttctagatgccagtggcaacatcaagattgctgattttggcctctccaacctgtatcacaaaggcaagttcctccagacgttctgtgggagccctctctatgcctcacctgagatcgtcaacgggaagccctatgtgggcccagaggtggacagctggtctctgggcgttcttctgtacatcctggtgcatggcaccatgccctttgacgggcaggatcataaaaccctggtgaaacaaatcagtagcggggcttaccgagagccgtgcaaaccgtctgatgcctgtggcctgatccggtggctgttaatggtgaatcccatccgtcgggccactctggaggatgtagccagtcattggtgggtcaactggggttacagcacccgaattggggaacaggaagctctgcgagagggtgggcaccctagcggtgactctggccgggcctctatggcggactggttacgtcgctcctcccgccccctcctggagaatggagccaaagtgtgtagcttcttcaagcagcatgtgccgggaggtggaagcacgggaccggggctggagcggcaacattctcttaagaagtcccgcaaggagaatgacatggctcagactctgcagaatgacccagttgaagatacttcctctcgccctggcaagaacagcctcaagcttccgaaaggtatcctcaagaaaaagg

SEQ ID NO: 14 - DNA, 436 bp, RAT
ctgctcccaaggaagggcattcttaagaagtctcggcagcgtgaatctggttactactcctctccagagcccagtgagtctggggaactcttagacgcaggtgatgtgtttgtgagtggggaccccgtggagcagaagtctccacaagcttcagggcgcctccatcgcaagggcatcctcaaactcaatggcaagttttcccgcacagccttagaaggcactgcccctagcacctttggctccctagaccaactggcctcccctcatcctacagcccgggccagccgtccctcgggagctgtgagtgaggacagcatcctgtcctccgagtcctttgaccaattggacttgcccgagcggcttcccgaaaccccactgaggagctgtgtgtctgtggacaacctgaggaggcttgagcagcctccctcagaaggcc

SEQ ID NO: 15 - DNA, 300 bp, RAT
atgaagaagcaggcggtgaagcggcaccatcacaaacacaacctgaggcaccgctacgagttcctggagaccctgggcaagggcacctacgggaaggtgaagaaagcacgagagagctcgggacgcctggtggccatcaagtctatcaggaaggacaaaatcaaagatgagcaggatctgttgcacataaggagggagatcgagatcatgtcttcactcaaccacccccacatcattgccatccatgaagtgtttgagaacagcagcaagattgtgattgtcatggagtacgccagccga

SEQ ID NO: 16 - DNA, 169 bp, RAT
ggcgatctgtacgattacatcagtgagcggccacggctgaatgagcgggacgccaggcatttcttccgacagatcgtgtccgccctgcactactgccaccagaacgggattgttcaccgggacctcaagctggagaacatccttctagatgccagtggcaacatcaaga

SEQ ID NO: 17 - DNA, 343 bp, RAT
gcccaaacctctgatgaagaagcaggcggtgaagcggcaccatcacaaacacaacctgaggcaccgctacgagttcctggagaccctgggcaagggcacctacgggaaggtgaagaaagcacgagagagctcgggacgcctggtggccatcaagtctatcaggaaggacaaaatcaaagatgagcaggatctgttgcacataaggagggagatcgagatcatgtcttcactcaaccacccccacatcattgccatccatgaagtgtttgagaacagcagcaagattgtgattgtcatggagtacgccagccgaggcgatctgtacgattacatcagtgagcgg

SEQ ID NO: 18 - DNA, 361 bp, RAT
gttcttctgtacatcctggtgcatggcaccatgccctttgacgggcaggatcataaaaccctggtgaaacaaatcagtagcggggcttaccgagagccgtgcaaaccgtctgatgcctgtggcctgatccggtggctgttaatggtgaatcccatccgtcgggccactctggaggatgtagccagtcattggtgggtcaactggggttacagcacccgaattggggaacaggaagctctgcgagagggtgggcaccctagcggtgactctggccgggcctctatggcggactggttacgtcgctcctcccgccccctcctggagaatggagccaaagtgtgtagcttcttcaagcagcatg

SEQ ID NO: 19 - DNA, 280 bp, RAT
gcatttcttccgacagatcgtgtccgccctgcactactgccaccagaacgggattgttcaccgggacctcaagctggagaacatccttctagatgccagtggcaacatcaagattgctgattttggcctctccaacctgtatcacaaaggcaagttcctccagacgttctgtgggagccctctctatgcctcacctgagatcgtcaacgggaagccctatgtgggcccagaggtggacagctggtctctgggcgttcttctgtacatcctggtgcatggc

SEQ ID NO: 20 - DNA, 77 bp, RAT
cacccccacatcattgccatccatgaagtgtttgagaacagcagcaagattgtgattgtcatggagtacgccagccg

SEQ ID NO: 21 - DNA, 35 bp, RAT
ctgatgcctgtggcctgatccggtggctgttaatg

SEQ ID NO: 22 - DNA, 270 bp, RAT
tggagaccctgggcaagggcacctacgggaaggtgaagaaagcacgagagagctcgggacgcctggtggccatcaagtctatcaggaaggacaaaatcaaagatgagcaggatctgttgcacataaggagggagatcgagatcatgtcttcactcaaccacccccacatcattgccatccatgaagtgtttgagaacagcagcaagattgtgattgtcatggagtacgccagccgaggcgatctgtacgattacatcagtgagcggccac

SEQ ID NO: 23 - DNA, 226 bp, RAT
tatcacaaaggcaagttcctccagacgttctgtgggagccctctctatgcctcacctgagatcgtcaacgggaagccctatgtgggcccagaggtggacagctggtctctgggcgttcttctgtacatcctggtgcatggcaccatgccctttgacgggcaggatcataaaaccctggtgaaacaaatcagtagcggggcttaccgagagccgtgcaaaccgtctg

SEQ ID NO: 24 - DNA, 152 bp, RAT
tgtttgagaacagcagcaagattgtgattgtcatggagtacgccagccgaggcgatctgtacgattacatcagtgagcggccacggctgaatgagcgggacgccaggcatttcttccgacagatcgtgtccgccctgcactactgccaccag

SEQ ID NO: 25 - DNA, 66 bp, RAT
aacgggattgttcaccgggacctcaagctggagaacatccttctagatgccagtggcaacatcaag

SEQ ID NO: 26 - DNA, 123 bp, RAT
attgctgattttggcctctccaacctgtatcacaaaggcaagttcctccagacgttctgtgggagccctctctatgcctcacctgagatcgtcaacgggaagccctatgtgggcccagaggtg

SEQ ID NO: 27 - DNA, 2026 bp, RAT
gtgacctctgagcctgcggctctccgcgcgctgctgctgctgcccgaccccctccgcctcgccgtccccgcaccatggagtcggtggccttacaccggcgcgggaacctggctccctcggcctccgccctggccacggagagcgcccggccgctggcggaccggctcatcaagtcgcccaaacctctgatgaagaagcaggcggtgaagcggcaccatcacaaacacaacctgaggcaccgctacgagttcctggagaccctgggcaagggcacctacgggaaggtgaagaaagcacgagagagctcgggacgcctggtggccatcaagtctatcaggaaggacaaaatcaaagatgagcaggatctgttgcacataaggagggagatcgagatcatgtcttcactcaaccacccccacatcattgccatccatgaagtgtttgagaacagcagcaagattgtgattgtcatggagtacgccagccgaggcgatctgtacgattacatcagtgagcggccacggctgaatgagcgggacgccaggcatttcttccgacagatcgtgtccgccctgcactactgccaccagaacgggattgttcaccgggacctcaagctggagaacatccttctagatgccagtggcaacatcaagattgctgattttggcctctccaacctgtatcacaaaggcaagttcctccagacgttctgtgggagccctctctatgcctcacctgagatcgtcaacgggaagccctatgtgggcccagaggtggacagctggtctctgggcgttcttctgtacatcctggtgcatggcaccatgccctttgacgggcaggatcataaaaccctggtgaaacaaatcagtagcggggcttaccgagagccgtgcaaaccgtctgatgcctgtggcctgatccggtggctgttaatggtgaatcccatccgtcgggccactctggaggatgtagccagtcattggtgggtcaactggggttacagcacccgaattggggaacaggaagctctgcgagagggtgggcaccctagcggtgactctggccgggcctctatggcggactggttacgtcgctcctcccgccccctcctggagaatggagccaaagtgtgtagcttcttcaagcagcatgtgccgggaggtggaagcacgggaccggggctggagcggcaacattctcttaagaagtcccgcaaggagaatgacatggctcagactctgcagaatgacccagttgaagatacttcctctcgccctggcaagaacagcctcaagcttccgaaaggtatcctcaagaaaaaggcctctccctcatcgggggaggtacaggagggccctcaggaactcagaccagtgtccaataccccagggcagcctgtccctgctatacccctgctcccaaggaagggcattcttaagaagtctcggcagcgtgaatctggttactactcctctccagagcccagtgagtctggggaactcttagacgcaggtgatgtgtttgtgagtggggaccccgtggagcagaagtctccacaagcttcagggcgcctccatcgcaagggcatcctcaaactcaatggcaagttttcccgcacagccttagaaggcactgcccctagcacctttggctccctagaccaactggcctcccctcatcctacagcccgggccagccgtccctcgggagctgtgagtgaggacagcatcctgtcctccgagtcctttgaccaattggacttgcccgagcggcttcccgaaaccccactgaggagctgtgtgtctgtggacaacctgaggaggcttgagcagcctccctcagaaggcctaaaacgatggtggcaggaatccttgggggatagccgcttttctctgacagactgccaagaggtgacagcagcctacagacaagccctaggaatctgctcgaagctcagctgaggaggagaggcagtgccccagtgatggggtagactcttagaggggtttgcagaggaacc

SEQ ID NO: 28 - DNA, 177 bp, RAT
cacataagttcctgttcgcatcgaccaccagggttagaaccctgacatccctggaagtaatgtggagcaacctcgcttatttaaagaggaaacagcctctggtttccatctctgctgctgtgcatctcaaagacctagaaagactcaactgctgtttcacttcatctcaaggggacc

SEQ ID NO: 29 - DNA, 197 bp, RAT
aagtgaattttgctgctttcggtaatgtgaatgctgtgttctggggaaagccactgtgtcattgaagtgtgtgtacagagaagtatttggcagtgattccttctaatggggggtggccttttcagatgtatgtcttgagcactgtctggattgggtctcctgtcccctcacaccagaggctgtccaccctccctcat

SEQ ID NO: 30 - DNA, 94 bp, RAT
ctcagagacctgagccttgaagctgttcctgataaccagactatgatggatatgtctgtttctcaggccagcaggacccagaatgtgctgactt

SEQ ID NO: 31 - DNA, 712 bp, RAT
agcaagattgtgattgtcatggagtacgccagccgaggcgatctgtacgattacatcagtgagcggccacggctgaatgagcgggacgccaggcatttcttccgacagatcgtgtccgccctgcactactgccaccagaacgggattgttcaccgggacctcaagctggagaacatccttctagatgccagtggcaacatcaagattgctgattttggcctctccaacctgtatcacaaaggcaagttcctccagacgttctgtgggagccctctctatgcctcacctgagatcgtcaacgggaagccctatgtgggcccagaggtggacagctggtctctgggcgttcttctgtacatcctggtgcatggcaccatgccctttgacgggcaggatcataaaaccctggtgaaacaaatcagtagcggggcttaccgagagccgtgcaaaccgtctgatgcctgtggcctgatccggtggctgttaatggtgaatcccatccgtcgggccactctggaggatgtagccagtcattggtgggtcaactggggttacagcacccgaattggggaacaggaagctctgcgagagggtgggcaccctagcggtgactctggccgggcctctatggcggactggttacgtcgctcctcccgccccctcctggagaatggagccaaagtgtgtagcttcttcaagcagcatgtgccgg

SEQ ID NO: 32 - DNA, 741 bp, RAT
tgacctctgagcctgcggctctccgcgcgctgctgctgctgcccgaccccctccgcctcgccgtccccgcaccatggagtcggtggccttacaccggcgcgggaacctggctccctcggcctccgccctggccacggagagcgcccggccgctggcggaccggctcatcaagtcgcccaaacctctgatgaagaagcaggcggtgaagcggcaccatcacaaacacaacctgaggcaccgctacgagttcctggagaccctgggcaagggcacctacgggaaggtgaagaaagcacgagagagctcgggacgcctggtggccatcaagtctatcaggaaggacaaaatcaaagatgagcaggatctgttgcacataaggagggagatcgagatcatgtcttcactcaaccacccccacatcattgccatccatgaagtgtttgagaacagcagcaagattgtgattgtcatggagtacgccagccgaggcgatctgtacgattacatcagtgagcggccacggctgaatgagcgggacgccaggcatttcttccgacagatcgtgtccgccctgcactactgccaccagaacgggattgttcaccgggacctcaagctggagaacatccttctagatgccagtggcaacatcaagattgctgattttggcctctccaacctgtatcacaaaggcaagttcctccagacgttctgtgggagccctctctatgcctcacctga

SEQ ID NO: 33 - DNA, 660 bp, RAT
ggtgaatcccatccgtcgggccactctggaggatgtagccagtcattggtgggtcaactggggttacagcacccgaattggggaacaggaagctctgcgagagggtgggcaccctagcggtgactctggccgggcctctatggcggactggttacgtcgctcctcccgccccctcctggagaatggagccaaagtgtgtagcttcttcaagcagcatgtgccgggaggtggaagcacgggaccggggctggagcggcaacattctcttaagaagtcccgcaaggagaatgacatggctcagactctgcagaatgacccagttgaagatacttcctctcgccctggcaagaacagcctcaagcttccgaaaggtatcctcaagaaaaaggcctctccctcatcgggggaggtacaggagggccctcaggaactcagaccagtgtccaataccccagggcagcctgtccctgctatacccctgctcccaaggaagggcattcttaagaagtctcggcagcgtgaatctggttactactcctctccagagcccagtgagtctggggaactcttagacgcaggtgatgtgtttgtgagtggggaccccgtggagcagaagtctccacaagcttcagggcgcctccatcgcaagggcatcctcaa

SEQ ID NO: 34 - DNA, 521 bp, RAT
tctgagcctgcggctctccgcgcgctgctgctgctgcccgaccccctccgcctcgccgtccccgcaccatggagtcggtggccttacaccggcgcgggaacctggctccctcggcctccgccctggccacggagagcgcccggccgctggcggaccggctcatcaagtcgcccaaacctctgatgaagaagcaggcggtgaagcggcaccatcacaaacacaacctgaggcaccgctacgagttcctggagaccctgggcaagggcacctacgggaaggtgaagaaagcacgagagagctcgggacgcctggtggccatcaagtctatcaggaaggacaaaatcaaagatgagcaggatctgttgcacataaggagggagatcgagatcatgtcttcactcaaccacccccacatcattgccatccatgaagtgtttgagaacagcagcaagattgtgattgtcatggagtacgccagccgaggcgatctgtacgattacatcagtgagcggccacggctg

SEQ ID NO: 35 - DNA, 579 bp, RAT
ataccccagggcagcctgtccctgctatacccctgctcccaaggaagggcattcttaagaagtctcggcagcgtgaatctggttactactcctctccagagcccagtgagtctggggaactcttagacgcaggtgatgtgtttgtgagtggggaccccgtggagcagaagtctccacaagcttcagggcgcctccatcgcaagggcatcctcaaactcaatggcaagttttcccgcacagccttagaaggcactgcccctagcacctttggctccctagaccaactggcctcccctcatcctacagcccgggccagccgtccctcgggagctgtgagtgaggacagcatcctgtcctccgagtcctttgaccaattggacttgcccgagcggcttcccgaaaccccactgaggagctgtgtgtctgtggacaacctgaggaggcttgagcagcctccctcagaaggcctaaaacgatggtggcaggaatccttgggggatagccgcttttctctgacagactgccaagaggtgacagcagcctacagacaagccctaggaatctgctcgaagctcagct

SEQ ID NO: 36 - DNA, 548 bp, RAT
cctgctatacccctgctcccaaggaagggcattcttaagaagtctcggcagcgtgaatctggttactactcctctccagagcccagtgagtctggggaactcttagacgcaggtgatgtgtttgtgagtggggaccccgtggagcagaagtctccacaagcttcagggcgcctccatcgcaagggcatcctcaaactcaatggcaagttttcccgcacagccttagaaggcactgcccctagcacctttggctccctagaccaactggcctcccctcatcctacagcccgggccagccgtccctcgggagctgtgagtgaggacagcatcctgtcctccgagtcctttgaccaattggacttgcccgagcggcttcccgaaaccccactgaggagctgtgtgtctgtggacaacctgaggaggcttgagcagcctccctcagaaggcctaaaacgatggtggcaggaatccttgggggatagccgcttttctctgacagactgccaagaggtgacagcagcctacagacaagccctaggaatctgctc

SEQ ID NO: 37 - DNA, 585 bp, RAT
gaacaggaagctctgcgagagggtgggcaccctagcggtgactctggccgggcctctatggcggactggttacgtcgctcctcccgccccctcctggagaatggagccaaagtgtgtagcttcttcaagcagcatgtgccgggaggtggaagcacgggaccggggctggagcggcaacattctcttaagaagtcccgcaaggagaatgacatggctcagactctgcagaatgacccagttgaagatacttcctctcgccctggcaagaacagcctcaagcttccgaaaggtatcctcaagaaaaaggcctctccctcatcgggggaggtacaggagggccctcaggaactcagaccagtgtccaataccccagggcagcctgtccctgctatacccctgctcccaaggaagggcattcttaagaagtctcggcagcgtgaatctggttactactcctctccagagcccagtgagtctggggaactcttagacgcaggtgatgtgtttgtgagtggggaccccgtggagcagaagtctccacaagcttcagggcgcctccatcgcaagggcatcctcaaactcaat

SEQ ID NO: 38 - DNA, 331 bp, RAT
acatcctggtgcatggcaccatgccctttgacgggcaggatcataaaaccctggtgaaacaaatcagtagcggggcttaccgagagccgtgcaaaccgtctgatgcctgtggcctgatccggtggctgttaatggtgaatcccatccgtcgggccactctggaggatgtagccagtcattggtgggtcaactggggttacagcacccgaattggggaacaggaagctctgcgagagggtgggcaccctagcggtgactctggccgggcctctatggcggactggttacgtcgctcctcccgccccctcctggagaatggagccaaagtgtg

SEQ ID NO: 39 - DNA, 164 bp, RAT
tggagaccctgggcaagggcacctacgggaaggtgaagaaagcacgagagagctcgggacgcctggtggccatcaagtctatcaggaaggacaaaatcaaagatgagcaggatctgttgcacataaggagggagatcgagatcatgtcttcactcaaccacccc

SEQ ID NO: 40 - DNA, 261 bp, RAT
ggagccctctctatgcctcacctgagatcgtcaacgggaagccctatgtgggcccagaggtggacagctggtctctgggcgttcttctgtacatcctggtgcatggcaccatgccctttgacgggcaggatcataaaaccctggtgaaacaaatcagtagcggggcttaccgagagccgtgcaaaccgtctgatgcctgtggcctgatccggtggctgttaatggtgaatcccatccgtcgggccactctggaggatgtag

SEQ ID NO: 41 - protein, 630 aa, RAT
MESVALHRRGNLAPSASALATESARPLADRLIKSPKPLMKKQAVKRHHHKHNLRHRYEFLETLGKGTYGKVKKARESSGRLVAIKSIRKDKIKDEQDLLHIRREIEIMSSLNHPHIIAIHEVFENSSKIVIVMEYASRGDLYDYISERPRLNERDARHFFRQIVSALHYCHQNGIVHRDLKLENILLDASGNIKIADFGLSNLYHKGKFLQTFCGSPLYASPEIVNGKPYVGPEVDSWSLGVLLYILVHGTMPFDGQDHKTLVKQISSGAYREPCKPSDACGLIRWLLMVNPIRRATLEDVASHWWVNWGYSTRIGEQEALREGGHPSGDSGRASMADWLRRSSRPLLENGAKVCSFFKQHVPGGGSTGPGLERQHSLKKSRKENDMAQTLQNDPVEDTSSRPGKNSLKLPKGILKKKASPSSGEVQEGPQELRPVSNTPGQPVPAIPLLPRKGILKKSRQRESGYYSSPEPSESGELLDAGDVFVSGDPVEQKSPQASGRLHRKGILKLNGKFSRTALEGTAPSTFGSLDQLASPHPTARASRPSGAVSEDSILSSESFDQLDLPERLPETPLRSCVSVDNLRRLEQPPSEGLKRWWQESLGDSRFSLTDCQEVTAAYRQALGICSKLS

SEQ ID NO: 42 - protein, 59 aa, RAT
ARPLADRLIKSPKPLMKKQAVKRHHHKHNLRHRYEFLETLGKGTYGKVKKARESSGRLV

SEQ ID NO: 43 - protein, 40 aa, RAT
VAIKSIRKDKIKDEQDLLHIRREIEIMSSLNHPHIIAIHE

SEQ ID NO: 44 - protein, 49 aa, RAT
VFENSSKIVIVMEYASRGDLYDYISERPRLNERDARHFFRQIVSALHYC

SEQ ID NO: 45 - protein, 35 aa, RAT
IADFGLSNLYHKGKFLQTFCGSPLYASPEIVNGKP

SEQ ID NO: 46 - protein, 39 aa, RAT
VDSWSLGVLLYILVHGTMPFDGQDHKTLVKQISSGAYRE

SEQ ID NO: 47 - protein, 140 aa, RAT
DACGLIRWLLMVNPIRRATLEDVASHWWVNWGYSTRIGEQEALREGGHPSGDSGRASMADWLRRSSRPLLENGAKVCSFFKQHVPGGGSTGPGLERQHSLKKSRKENDMAQTLQNDPVEDTSSRPGKNSLKLPKGILKKK

SEQ ID NO: 48 - protein, 146 aa, RAT
PLLPRKGILKKSRQRESGYYSSPEPSESGELLDAGDVFVSGDPVEQKSPQASGRLHRKGILKLNGKFSRTALEGTAPSTFGSLDQLASPHPTARASRPSGAVSEDSILSSESFDQLDLPERLPETPLRSCVSVDNLRRLEQPPSEG

SEQ ID NO: 49 - protein, 15 aa, RAT
LGDSRFSLTDCQEVT

SEQ ID NO: 50 - protein, 251 aa, RAT
YEFLETLGKGTYGKVKKARESSGRLVAIKSIRKDKIKDEQDLLHIRREIEIMSSLNHPHIIAIHEVFENSSKIVIVMEYASRGDLYDYISERPRLNERDARHFFRQIVSALHYCHQNGIVHRDLKLENILLDASGNIKIADFGLSNLYHKGKFLQTFCGSPLYASPEIVNGKPYVGPEVDSWSLGVLLYILVHGTMPFDGQDHKTLVKQISSGAYREPCKPSDACGLIRWLLMVNPIRRATLEDVASHWWV

SEQ ID NO: 51 - DNA, 177 bp, Homo sapiens
gcccggccgctggcggaagggctgatcaagtcgcccaagcccctaatgaagaagcaggcggtgaagcggcaccaccacaagcacaacctgcggcaccgctacgagttcctggagaccctgggcaaaggcacctacgggaaggtgaagaaggcgcgggagagctcggggcgcctggtg

SEQ ID NO: 52 - DNA, 122 bp, Homo sapiens
ggtggccatcaagtcaatccggaaggacaaaatcaaagatgagcaagatctgatgcacatacggagggagattgagatcatgtcatcactcaaccaccctcacatcattgccatccatgaag

SEQ ID NO: 53 - DNA, 150 bp, Homo sapiens
agtgtttgagaacagcagcaagatcgtgatcgtcatggagtatgccagccggggcgacctttatgactacatcagcgagcggcagcagctcagtgagcgcgaagctaggcatttcttccggcagatcgtctctgccgtgcactattgcca

SEQ ID NO: 54 - DNA, 109 bp, Homo sapiens
agattgctgacttcggtctctccaacctctaccatcaaggcaagttcctgcagacattctgtgggagccccctctatgcctcgccagagattgtcaatgggaagcccta

SEQ ID NO: 55 - DNA, 121 bp, Homo sapiens
aggtggacagctggtccctgggtgttctcctctacatcctggtgcatggcaccatgccctttgatgggcatgaccataagatcctagtgaaacagatcagcaacggggcctaccgggagcc

SEQ ID NO: 56 - DNA, 421 bp, Homo sapiens
gatgcctgtggcctgatccggtggctgttgatggtgaaccccacccgccgggccaccctggaggatgtggccagtcactggtgggtcaactggggctacgccacccgagtgggagagcaggaggctccgcatgagggtgggcaccctggcagtgactctgcccgcgcctccatggctgactggctccggcgttcctcccgccccctcctggagaatggggccaaggtgtgcagcttcttcaagcagcatgcacctggtgggggaagcaccacccctggcctggagcgccagcattcgctcaagaagtcccgcaaggagaatgacatggcccagtctctccacagtgacacggctgatgacactgcccatcgccctggcaagagcaacctcaagctgccaaagggcattctcaagaagaagg

SEQ ID NO: 57 - DNA, 442 bp, Homo sapiens
cccctgctccccaagaagggcattctcaagaagccccgacagcgcgagtctggctactactcctctcccgagcccagtgaatctggggagctcttggacgcaggcgacgtgtttgtgagtggggatcccaaggagcagaagcctccgcaagcttcagggctgctcctccatcgcaaaggcatcctcaaactcaatggcaagttctcccagacagccttggagctcgcggcccccaccaccttcggctccctggatgaactcgccccacctcgccccctggcccgggccagccgaccctcaggggctgtgagcgaggacagcatcctgtcctctgagtcctttgaccagctggacttgcctgaacggctcccagagcccccactgcggggctgtgtgtctgtggacaacctcacggggcttgaggagcccccctcagagggcc

SEQ ID NO: 58 - DNA, 47 bp, Homo sapiens
ttgggggacagctgcttttccctgacagactgccaggaggtgacagc

SEQ ID NO: 59 - DNA, 56 bp, Homo sapiens
aaagtgaatcttgctgttttcaataatgtgaatgctatgttctgggaaaatccact

SEQ ID NO: 60 - DNA, 11 bp, Homo sapiens
cacaggcccag

SEQ ID NO: 61 - DNA, 13 bp, Homo sapiens
acctaaaccctct

SEQ ID NO: 62 - protein, 59 aa, Homo sapiens
ARPLAEGLIKSPKPLMKKQAVKRHHHKHNLRHRYEFLETLGKGTYGKVKKARESSGRLV

SEQ ID NO: 63 - protein, 40 aa, Homo sapiens
VAIKSIRKDKIKDEQDLMHIRREIEIMSSLNHPHIIAIHE

SEQ ID NO: 64 - protein, 49 aa, Homo sapiens
VFENSSKIVIVMEYASRGDLYDYISERQQLSEREARHFFRQIVSAVHYC

SEQ ID NO: 65 - protein, 35 aa, Homo sapiens
IADFGLSNLYHQGKFLQTFCGSPLYASPEIVNGKP

SEQ ID NO: 66 - protein, 39 aa, Homo sapiens
VDSWSLGVLLYILVHGTMPFDGHDHKILVKQISNGAYRE

SEQ ID NO: 67 - protein, 140 aa, Homo sapiens
DACGLIRWLLMVNPTRRATLEDVASHWWVNWGYATRVGEQEAPHEGGHPGSDSARASMADWLRRSSRPLLENGAKVCSFFKQHAPGGGSTTPGLERQHSLKKSRKENDMAQSLHSDTADDTAHRPGKSNLKLPKGILKKK

SEQ ID NO: 68 - protein, 147 aa, Homo sapiens
PLLPKKGILKKPRQRESGYYSSPEPSESGELLDAGDVFVSGDPKEQKPPQASGLLLHRKGILKLNGKFSQTALELAAPTTFGSLDELAPPRPLARASRPSGAVSEDSILSSESFDQLDLPERLPEPPLRGCVSVDNLTGLEEPPSEG

SEQ ID NO: 69 - protein, 15 aa, Homo sapiens
LGDSCFSLTDCQEVT

SEQ ID NO: 70 - DNA, 1186 bp, Homo sapiens
gcccggccgctggcggaagggctgatcaagtcgcccaagcccctaatgaagaagcaggcggtgaagcggcaccaccacaagcacaacctgcggcaccgctacgagttcctggagaccctgggcaaaggcacctacgggaaggtgaagaaggcgcgggagagctcggggcgcctggtggccatcaagtcaatccggaaggacaaaatcaaagatgagcaagatctgatgcacatacggagggagattgagatcatgtcatcactcaaccaccctcacatcattgccatccatgaagtgtttgagaacagcagcaagatcgtgatcgtcatggagtatgccagccggggcgacctttatgactacatcagcgagcggcagcagctcagtgagcgcgaagctaggcatttcttccggcagatcgtctctgccgtgcactattgccatcagaacagagttgtccaccgagatctcaagctggagaacatcctcttggatgccaatgggaatatcaagattgctgacttcggcctctccaacctctaccatcaaggcaagttcctgcagacattctgtgggagccccctctatgcctcgccagagattgtcaatgggaagccctacacaggcccagaggtggacagctggtccctgggtgttctcctctacatcctggtgcatggcaccatgccctttgatgggcatgaccataagatcctagtgaaacagatcagcaacggggcctaccgggagccacctaaaccctctgatgcctgtggcctgatccggtggctgttgatggtgaaccccacccgccgggccaccctggaggatgtggccagtcactggtgggtcaactggggctacgccacccgagtgggagagcaggaggctccgcatgagggtgggcaccctggcagtgactctgcccgcgcctccatggctgactggctccggcgttcctcccgccccctcctggagaatggggccaaggtgtgcagcttcttcaagcagcatgcacctggtgggggaagcaccacccctggcctggagcgccagcattcgctcaagaagtcccgcaaggagaatgacatggcccagtctctccacagtgacacggctgatgacactgcccatcgccctggcaagagcaacctcaagctgccaaagggcattctcaagaagaagg

SEQ ID NO: 71 - DNA, 439 bp, Homo sapiens
ctgctccccaagaagggcattctcaagaagccccgacagcgcgagtctggctactactcctctcccgagcccagtgaatctggggagctcttggacgcaggcgacgtgtttgtgagtggggatcccaaggagcagaagcctccgcaagcttcagggctgctcctccatcgcaaaggcatcctcaaactcaatggcaagttctcccagacagccttggagctcgcggcccccaccaccttcggctccctggatgaactcgccccacctcgccccctggcccgggccagccgaccctcaggggctgtgagcgaggacagcatcctgtcctctgagtcctttgaccagctggacttgcctgaacggctcccagagcccccactgcggggctgtgtgtctgtggacaacctcacggggcttgaggagcccccctcagagggcc

SEQ ID NO: 72 - DNA, 47 bp, Homo sapiens; identical to SEQ ID NO: 58

SEQ ID NO: 73 - DNA, 56 bp, Homo sapiens; identical to SEQ ID NO: 59

SEQ ID NO: 74 - DNA, 300 bp, Homo sapiens
atgaagaagcaggcggtgaagcggcaccaccacaagcacaacctgcggcaccgctacgagttcctggagaccctgggcaaaggcacctacgggaaggtgaagaaggcgcgggagagctcggggcgcctggtggccatcaagtcaatccggaaggacaaaatcaaagatgagcaagatctgatgcacatacggagggagattgagatcatgtcatcactcaaccaccctcacatcattgccatccatgaagtgtttgagaacagcagcaagatcgtgatcgtcatggagtatgccagccgg

SEQ ID NO: 75 - DNA, 169 bp, Homo sapiens
ggcgacctttatgactacatcagcgagcggcagcagctcagtgagcgcgaagctaggcatttcttccggcagatcgtctctgccgtgcactattgccatcagaacagagttgtccaccgagatctcaagctggagaacatcctcttggatgccaatgggaatatcaaga

SEQ ID NO: 76 - DNA, 341 bp, Homo sapiens
gcccaagcccctaatgaagaagcaggcggtgaagcggcaccaccacaagcacaacctgcggcaccgtacgagttcctggagaccctgggcaaaggcacctacgggaaggtgaagaaggcgcgggagagctcggggcgcctggtggccatcaagtcaatccggaagacaaaatcaaagatgagcaagatctgatgcacatacggagggagattgagatcatgtcatcactcaaccaccctcacatcattgccatccatgaagtgtttgagaacagcagcaagatcgtgatcgtcatggagtatgccagccggggcgacctttatgactacatcagcgagcgg

SEQ ID NO: 77 - DNA, 356 bp, Homo sapiens
gttctcctctacatcctggtgcatggcaccatgcccttgatgggcatgaccataagatcctagtgaaacgagatcagcaacggggcctaccgggagcccacctaaacgctctgatgcctgtggcctgatccggtggctgttgatggtgaaccccagccgccgggcaccctggaggatgtggccagtcactggtgggtcaactggggctacgccacccgagtgggagagcaggaggctccgcatgagggtgggcaccctggcagtgactctgcccgcgctccatggctgactgctccggcgtcctcccgcccctcctgagaatggggccaaggtgtgcagcttcttcaagcagcatg

SEQ ID NO: 78 - DNA, 282 bp, Homo sapiens
gcatttcttccggcagatcgtctctgccgtgcacctattgccatcagaacagagttgtccaccgagatctcaagctggagaacatcctcttggatgccaatgggaatatcaagattgctgacttcggcctctccaacctctaccatcaaggcaagttcctgcagacattctgtgggagccccctctatgcctcgccagagatttgtcaatgggaagcctacacaggcccagaggttggacagctggtccctgggtgttctcctctacatcctggtgcatggc

SEQ ID NO: 79 - DNA, 78 bp, Homo sapiens
caccctcacatcatctgccatccatgaagtgtttgagaacagcagcaagatcgtgatcgtcatggagtatgccagccg

SEQ ID NO: 80 - DNA, 35 bp, Homo sapiens
  misc_feature: “n” equal anyone of the the four possibilities a, c, t, or g
ctgatngctgtggcttgatccggtggctgttnatg

SEQ ID NO: 81 - DNA, 270 bp, Homo sapiens
tggagaccctgggcaaaggcacctacgggaaggtgaagaaggcgcgggagagctcggggcgcctggtggccatcaagtcaatccggaaggacaaaatcaaagatgagcaagatctgatgcacatacggagggagattgagatcatgtcatcactcaaccaccctcacatcattgccatccatgaagtgtttgagaacagcagcaagatcgtgatcgtcatggagtatgccagcgggggcgacttttatgactacgtcagcggggcggcag

SEQ ID NO: 82 - DNA, 226 bp, Homo sapiens
taccatcaaggcaagttcctgcagacattctgtgggagccccctctatgcctcgccagagattgtcaatgggaagccctacacaggcccagaggtggacagctggtccctgggtgttctcctctacatccctgtgcatggcaccatgccctttgatgggcatgaccataagatcctagtgaaacagatcagcaacggggcctaccgggagccaactaaaccctctg

SEQ ID NO: 83 - DNA, 122 bp, MOUSE
ggtggccatcaagtccatcaggaaagacaaaatcaaagatgagcaggatctgctgcacatacggagggagattgagatcatgtcttcactcaaccacccccacatcattgccatccatgaag

SEQ ID NO: 84 - DNA, 152 bp, MOUSE
tgtttgagaatagcagcaagattgtgattgtcatggagtatgccagccgaggcgatctgtatgattacatcagtgagcggccacggctgagtgagcgggacgccaggcatttcttccgacagatcgtgtctgccctgcactactgccaccag

SEQ ID NO: 85 - DNA, 66 bp, MOUSE
aacgggatcgttcaccgagatctcaagctggaaaacatccttctagatgccaatggaaacatcaag

SEQ ID NO: 86 - DNA, 123 bp, MOUSE
attgctgactttggcctctccaacctgtaccacaaaggcaagttcctccagacgttctgtgggagccctctctacgcctcgcctgagatagtcaacgggaagccctatgtgggcccagaggtg

SEQ ID NO: 87 - DNA, 2027 bp, MOUSE
gtgacctctgagcccgcggctcagcgcgcgctgctactgctgcccgacccactccacctcgcggtccccgcaccatggagtcggtggccttactccagcgcccgagccaggctccctcggcctccgccctggcctcggagagcgcccggccgctggcggacgggctcatcaagtcgcctaaacctctgatgaagaagcaggcggtgaagcggcaccatcacaaacacaacctgcggcaccgctacgagttcctggagacgctgggcaagggcacctacgggaaggtgaagaaggcacgagagagctcggggcgtctggtggccatcaagtccatcaggaaagacaaaatcaaagatgagcaggatctgttgcacataaggagggagatcgagatcatgtcttcactcaaccacccccacatcattgccatccatgaagtgtttgagaatagcagcaagattgtgattgtcatggagtatgccagccgaggcgatctgtatgattacatcagtgagcggccacggctgagtgagcgggacgccaggcatttcttccgacagatcgtgtctgccctgcactactgccaccagaacgggatcgttcaccgagatctcaagctggaaaacatccttctagatgccaatggaaacatcaagattgctgactttggcctctccaacctgtaccacaaaggcaagttcctccagacgttctgtgggagccctctctacgcctcgcctgagatagtcaacgggaagccctatgtgggcccagaggtggacagctggtctctgggcgttctcctgtacatcctggtgcatggcaccatgccctttgacgggcaggatcataaaacactggtgaagcaaatcagtaacggggcttaccgtgagccgcccaagccgtccgatgcctgtggcctgatccggtggctgttaatggtgaaccccacccgtcgggccacactggaggatgtagccagtcattggtgggtcaactggggttacaccaccggagtcggggaacaggaagccctgcgtgagggtgggcaccctagtggtgactttggccgggcctccatggcggactggttacgtcgctcctcgcgccccctcctggagaatggagccaaggtgtgcagcttcttcaagcagcacgtgccgggaggtggaagcactgtacctgggctggagcggcaacattctcttaagaagtcccgaaaggagaatgacatggctcaaaatctgcaaggtgacccggctgaggatacctcttctcgccctggcaagagcagccttaagcttccgaaaggcattctcaagaaaaagtcctctacctcgtcaggggaggtacaggaggaccctcaggaactcagaccggtgcctgatactccagggcagcctgtccctgctgtatccctgctcccaaggaaaggcatccttaagaagtctcgacagcgtgaatctggttactactcctctccagagcccagcgagtctggggaactcttagacgccagtgatgtgtttgtgagtggggaccccgtggagcagaagtctccacaggcttcagggctcctcctccaccgcaagggcattctcaaactcaatggcaagttctcccgcacagccttagaaggcactacccctagcacctttggctccctggaccaactggcctcctcccatcctgcagcccggcccagccgcccctcaggggctgtgagtgaggacagcatcctgtcctccgagtcctttgaccaattggacttgcctgaacgtcttcccgaaaccccactgaggggctgtgtgtctgtggacaacctgagggggcttgagcagcctccctcagaaggtctgaagcgatggtggcaggaatccttgggggatagctgcttttctctgacagactgccaagaggtgactgcagcctacagacaagccctaggaatctgctcaaagctcagctgaggaagggagatggtgccctagtatggggtaggctctgagagggtttgcagaggaacc

SEQ ID NO: 88 - DNA, 178 bp, MOUSE
cacataagtttctgtttccatcaaccaccagggttagaaccctgacttcctgggaggtaatgtgtagtgactgccattatttagagaggaaacagcctctggtttccatctctgctgctgtgcatctcaaagacctgggaagactcggaccgctgtttgacttcatctcaaggggacc

SEQ ID NO: 89 - DNA, 205 bp, MOUSE
aagtgaattttgctgctttcaataatgtgaatgctgtgttctggggaactccactgtgccactgaagtttatgtacagagaagtatttggcaatgatgtccctctattcaaggggggtgggggcgtttttcaaatgtatgtcttgagcactgtctggattgagtctccagtcccttcacacccaaggctggccaccctccctcat

SEQ ID NO: 90 - DNA, 93 bp, MOUSE
ctcagagacttgaaccttgaagctgttcctagtacccagatgtggatggatgctctgtttctcaggccaacgggacctagaatgtgctgactt

SEQ ID NO: 91 - DNA, 707 bp, MOUSE
agcaagattgtgattgtcatggagtatgccagccgaggcgatctgtatgattacatcagtgagcggccacggctgagtgagcgggacgccaggcatttcttccgacagatcgtgtctgccctgcactactgccaccagaacgggatcgttcaccgagatctcaagctggaaaacatccttctagatgccaatggaaacatcaagattgctgactttggcctctccaacctgtaccacaaaggcaagttcctccagacgttctgtgggagccctctctacgcctcgcctgagatagtcaacgggaagccctatgtgggcccagaggtggacagctggtctctgggcgttctcctgtacatcctggtgcatggcaccatgccctttgacgggcaggatcataaaacactggtgaagcaaatcagtaacggggcttaccgtgagccgcccaagccgtccgatgcctgtggcctgatccggtggctgttaatggtgaaccccacccgtcgggccacactggaggatgtagccagtcattggtgggtcaactggggttacaccaccggagtcggggaacaggaagccctgcgtgagggtgggcaccctagtggtgactttggcgggctccatggcggactggttacgtcgctctcgcgcccctcctggagaatgggccacagtgtgcagttcttcaagccagcacgtgccgg

SEQ ID NO: 92 - DNA, 734 bp, MOUSE
tgacctctgagcccgcggctcagcgcgcgctgctactgtgcccgaccactccacctcgcggtccccgcaccatggagtcggtggccttactccagcgcccgagccaggctccctcggcctccgccctggcctcggagagcgcccggccgctggcggacgggctcatcaagtcgcctaaacctctgatgaagaagcaggcggtgaagcggcaccatcacaaacacaacctgcggcaccgctacgagttcctggagacgctgggcaagggcacctacgggaaggtgaagaaggcacgagagagctcggggcgtctggtggccatcaagtccatcaggaaagacaaaatcaaagatgagcaggatctgctgcacatacggagggagatgagatcatgtcttcactcaaccacccccacatcattgccatccatgaagtgtttgagaatagcagcaagattgtgattgtcatggagtatgccagccgaggcgatctgtatgattacatcagtgagcggcacggctgagtgagcgggacgccaggcatttcttccgacagatcgtgtctgcctgcactactgccaccagaacgggatcgttcaccgagatctcaagctggaaaacatccttctagatgccaatggaaacatcaagatgctgacttgggctctccaaacctgtaccacaagggccagttgctccagacgtctgggggagccctctctacgcctcgcctga

SEQ ID NO: 93 - DNA, 661 bp, MOUSE
ggtgaaccccacccgtcgggccacactggaggatgtagccagtcattggtgggtcaactggggttacaccaccggagtcggggaacaggaagccctgcgtgagggtgggcaccctagtggtgactttggccgggcctccatggcggactggttacgtcgctcctcgcgccccctcctggagaatggagccaaggtgtgcagcttcttcaagcagcacgtgccgggaggtggaagcactgtacctgggctggagcggcaacattctcttaagaagtcccgaaaggagaatgacatggctcaaaatctgcaaggtgacccggctgaggatacctcttctcgccctggcaagagcagccttaagcttccgaaaggcattctcaagaaaaagtcctctacctcgtcaggggaggtacaggaggaccctcaggaactcagaccggtgcctgatactccagggcagcctgtccctgctgtatccctgctcccaaggaaaggcatccttaagaagtctcgacagcgtgaatctggttactactcctctccagagcccagcgagtctggggaactcttagacgccagtgatgtgtttgtgagtgggggccccgtggagcagaagtctccacaggcttcaggctctcctccaccgcaagggcattctcaa

SEQ ID NO: 94 - DNA, 521 bp, MOUSE
tctgagcccgcggctctccgcgcgctgctactgctgcccgacccactccacctcgcggtccccgcaccatggagtcggtggccttactccagcgcccgagccaggctccctcggcctccgccctggcctcggagagcgcccggccgctggcggacgggctcatcaagtcgcctaaacctctgatgaagaagcaggcggtgaagcggcaccatcacaaacacaacctgcggcaccgctacgagttcctggagaccctgggcaagggcacctacgggaaggtgaagaaggcacgagagagctcggggcgtctggtggccatcaagtcaatcaggaaagacaaaatcaaagatgagcaggatctgctgcacatacggagggagattgagatcatgtcttcactcaaccacccccacatcattgccatccatgaagtgtttgagaatagcagcaagattgtgattgtcatggagtatgccagccgaggcgatctgtacgattacatcagtgagcggccacggctg

SEQ ID NO: 95 - DNA, 578 bp, MOUSE
atactccagggcagcctgtccctgctgtatccctgctcccaaggaaaggcatccttaagaagtctcgacagcgtgaatctggttactactcctctccagagcccagcgagtctggggaactcttagacgccagtgatgtgttgtgagtggggaccccgtggagcagaagtctccacaggcttcagggctcctcctccaccgcaagggcattctcaaactcaatggcaagttctcccgcacagccttagaaggcactacccctagcacctttggctccctggaccaactggcctcctcccatcctgcagcccggccagccgcccctcaggggctgtgagtgaggacagcatcctgtcctccgagtcctttgaccaattggacttgcctgaacgtcttcccgaaaccccactgaggggctgtgtgtctgtggacaacctgagggggcttgagcagcctccctcagaaggtctgaagcgatggtggcaggaatccttgggggatagctgcttttctctgacagactgcaagaggtgactgcagctacagacaagccctaggaatctgctcaaagctcagct

SEQ ID NO: 96 - DNA, 548 bp, MOUSE
cctgctgtatccctgctcccaaggaaaggcatccttaagaagtctcgacagcgtgaatctggttactactcctctccagagcccagcgagtctggggaactcttagacgccagtgatgtgttgtgagtggggaccccgtggagcagaagtctccacaggcttcagggctcctcctccaccgcaagggcattctcaaactcaatggcaagttctcccgcacagccttagaaggcactacccctagcacctttggctccctggaccaactggctcctcccatcctgcagcccggcccagccgccctcaggggctgtgagtgaggacagcatcctgtcctccgagtcctttgaccaattggacttgcctgaacgtcttcccgaaaccccactgaggggctgtgtgtctgtggacaacctgagggggcttgagcagcctccctcagaaggtctgaagcgatggtggcaggaatccttgggggatagctgcttttctctgacagactgccaagaggtgactgcagcctacagacaagccctaggaatctgctc

SEQ ID NO: 97 - DNA, 588 bp, MOUSE
gaacaggaagccctgcgtgagggtgggcaccctagtggtgactttggccgggcctccatggcggactggttacgtcgctcctcgcgccccctcctggagaatggagccaaggtgtgcagcttcttcaagcagcacgtgccgggaggtggaagcactgtacctgggctggagcggcaacattctcttaagaagtcccgaaaggagaacgacatggctcaaaatctgcaaggtgacccggctgaggatacctcttctcgccctggcaagagcagccttaaacttccgaaaggcattctcaagaaaaagtcctctacctcgtcaggggaggtacaggaggaccctcaggaactcagaccggtgcctgatactccagggcagcctgtccctgctgtatccctgctcccaaggaaaggcatccttaagaagtctcgacagcgtgaatctggttactactcctctccagagcccagcgagtctggggaactcttagacgccagtgatgtgtttgtgagtggggaccccgtggagcagaagtccccacaggcttcagggctcctcctccaccgcaagggcattctcaaactcaat

SEQ ID NO: 98 - DNA, 331 bp, CLONE
acatcctggtgcatggcaccatgccctttgacgggcaggatcataaaacactggtgaagcaaatcagtaacggggcttaccgtgagccgcccaagccgtccgatgcctgtggcctgatccggtggctgttaatggtgaaccccacccgtcgggccacactggaggatgtagccagtcattggtgggtcaactggggttacaccaccggagtcggggaacaggaagccctgcgtgagggtgggcaccctagtggtgactttggccgggcctccatggcggactggttacgtcgctcctcgcgccccctcctggagaatggagccaaggtgtg

SEQ ID NO: 99 - DNA, 164 bp, MOUSE
tggagacgctgggcaagggcacctacgggaaggtgaagaaggcacgagagagctcggggcgtctggtggccatcaagtccatcaggaaagacaaaatcaaagatgagcaggatctgctgcacatacggagggagattgagatcatgtcttcactcaaccacccc

SEQ ID NO: 100 - DNA, 261 bp, MOUSE
ggagccctctctacgcctcgcctgagatagtcaacgggaagccctatgtgggcccagaggtggacagctggtctctgggcgttctcctgtacatcctggtgcatggcaccatgccctttgacgggcaggatcataaaacactggtgaagcaaatcagtaacggggcttaccgtgagccgcccaagccgtccgatgcctgtggcctgatccggtggctgttaatggtgaaccccacccgtcgggccacactggaggatgtag

SEQ ID NO: 101 - protein, 251 aa, Homo sapiens
YEFLETLGKGTYGKVKKARESSGRLVAIKSIRKDKIKDEQDLMHIRREIEIMSSLNHPHIIAIHEVFENSSKIVIVMEYASRGDLYDYISERQQLSEREARHFFRQIVSAVHYCHQNRVVHRDLKLENILLDANGNIKIADFGLSNLYHQGKFLQTFCGSPLYASPEIVNGKPYTGPEVDSWSLGVLLYILVHGTMPFDGHDHKILVKQISNGAYREPPKPSDACGLIRWLLMVNPTRRATLEDVASHWWV

SEQ ID NO: 102 - protein, 252 aa, Homo sapiens
YELQETLGKGTYGKVKRATERFSGRVVAIKSIRKDKIKDEQDMVHIRREIEIMSSLNHPHIISIYEVFENKDKIVIIMEYASKGELYDYISERRRLSERETRHFFRQIVSAVHYCHKNGVVHRDLKLENILLDDNCNIKIADFGLSNLYQKDKFLQTFCGSPLYASPEIVNGRPYRGPEVDSWALGVLLYTLVYGTMPFDGFDHKNLIRQISSGEYREPTQPSDARGLIRWMLMVNPDRRATIEDIANHWWV

SEQ ID NO: 103 - protein, 251 aa, Homo sapiens
YEFLETLGKGTYGKVKKARESSGRLVAIKSIRKDKIKDEQDLLHIRREIEIMSSLNHPHIIAIHEVFENSSKIVIVMEYASRGDLYDYISERPRLSERDARHFFRQIVSALHYCHQNGIVHRDLKLENILLDANGNIKIADFGLSNLYHKGKFLQTFCGSPLYASPEIVNGKPYVGPEVDSWSLGVLLYILVHGTMPFDGQDHKTLVKQISNGAYREPPKPSDACGLIRWLLMVNPTRRATLEDVASHWWV

SEQ ID NO: 104 - DNA, 29 bp, Artificial Sequence
  primer
ccggatccatggagtcggtggccttacac

SEQ ID NO: 105 - DNA, 28 bp, Artificial Sequence
  primer
ccggatccctaagagttccccagactca

SEQ ID NO: 106 - DNA, 21 bp, Artificial Sequence
  primer
tgaggcaccgctacgagttcc

SEQ ID NO: 107 - DNA, 21 bp, Artificial Sequence
  primer
accggatcaggccacaggcat

SEQ ID NO: 108 - DNA, 23 bp, Artificial Sequence
  primer
ccagttgacccaccaatgactgg

SEQ ID NO: 109 - protein, 15 aa, Artificial Sequence
  synthetic peptide substrate
HMRSAMSGLHLVKRR